(19) United States
(12) Patent Application Publication
(10) Pub. No.: US 2024/0368211 A1
(43) Pub. Date: Nov. 7, 2024

(54) PROCESSES OF MAKING ONAPRISTONE AND INTERMEDIATES THEREOF

(71) Applicants: CONTEXT BIOPHARMA INC., Philadelphia, PA (US); TYLIGAND BIOSCIENCE (SHANGHAI) LIMITED, Shanghai (CN)

(72) Inventors: Peter WUTS, Philadelphia, PA (US); William F. RENCHER, Philadelphia, PA (US); Tony ZHANG, Shanghai (CN); Boyu ZHONG, Shanghai (CN); Anjiang YANG, Shanghai (CN); Fei LV, Shanghai (CN); Qihua ZHAO, Shanghai (CN); Jiaoyang FENG, Shanghai (CN); Jifa WANG, Shanghai (CN)

(21) Appl. No.: 18/290,604

(22) PCT Filed: Jul. 19, 2022
(86) PCT No.: PCT/US2022/037569

§ 371 Date: Jan. 19, 2024

(30) Foreign Application Priority Data
Jul. 19, 2021 (WO) PCT/CN2021/107099

(51) Int. Cl. C07J 21/00; C07J 9/00

(52) U.S. Cl. CPC C07J 21/006; C07J 9/00

(57) ABSTRACT

The present disclosure describes novel processes and intermediates for making onapristone.

### FIELD

The present disclosure describes novel processes and intermediates for making onapristone.

### BACKGROUND

Onapristone (ONA, Formula VII) is an anti-progestin drug and progesterone receptor antagonist which was originally developed for contraceptive use. However, it has demonstrated substantial activity in advanced breast cancer. Currently, onapristone is under development for the treatment of prostate cancer, endometrial cancer, breast cancer, ovarian cancer and other progesterone receptor-positive gynecologic cancers.

Synthesis of onapristone has been reported previously in the field. The reported methods of preparation, however, lack efficiency due to the reasons such as poor selectivity and requirement for multiple and cumbersome chromatographic separations to afford material of pharmaceutically acceptable purity. What is needed is an improved, less costly method for making, forming, or synthesizing onapristone with fewer impurities and fewer and simpler steps. The methods and processes described and provided herein fulfill these needs as well as others.

### SUMMARY OF EMBODIMENTS

The present disclosure provides novel processes and intermediates for making the compounds of Formulae (II), (III), (IV), (V), (VI), (VII), (IX), (X), (XI), (XII), (II-a), (III-a), (IV-a), (V-a), (VI-a), (X-a), (XI-a), and (XII).

In some embodiments, processes of preparing compounds of Formula (II), or a pharmaceutically acceptable salt thereof are provided. In some embodiments, the processes comprise contacting the compound of

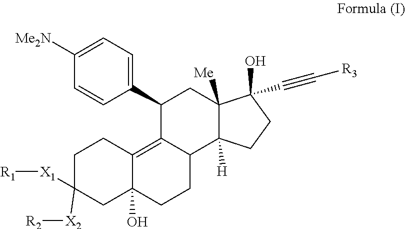

with a base in a first organic solvent under suitable conditions to produce the compound of

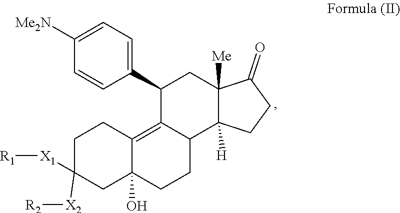

wherein R₁, R₂, R₃, X₁, and X₂ are as provided for herein and, for example, can be selected from the respective groups of chemical moieties described herein.

In some embodiments, methods or processes of preparing a compound of Formula (III) or a pharmaceutically acceptable salt thereof are provided. In some embodiments, the process comprises: irradiating a solution of the compound of

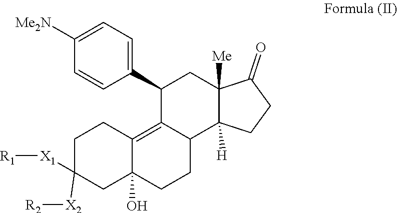

in an organic solvent with a UV light in a flow reactor to produce the compound of

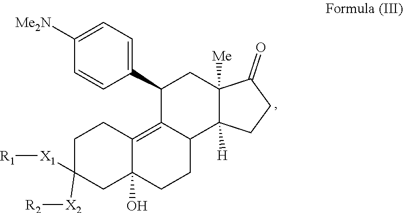

wherein R₁, R₂, X₁, and X₂ are as provided for herein and, for example, can be selected from the respective groups of chemical moieties described herein.

In some embodiments, methods or processes of preparing a compound of Formula (X), or a salt thereof, are provided. In some embodiments, the process comprises contacting the compound of

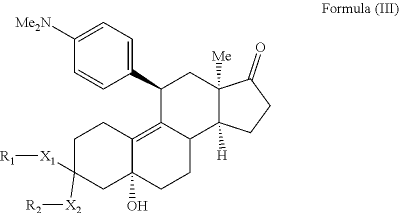

with an organometallic reagent having a formula of

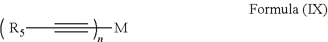

to produce the compound of

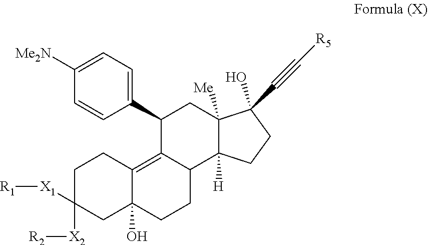

or a salt form thereof; wherein R₁, R₂, R₅, M, X₁, and X₂ are as provided for herein and, for example, can be selected from the respective groups of chemical moieties described herein. In some embodiments, the compound of Formula (X) or a salt form thereof, has a formula of

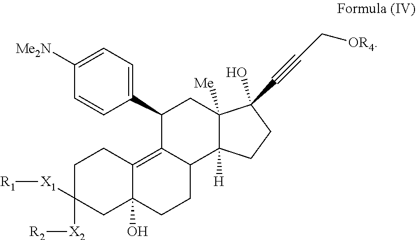

In some embodiments, methods or processes of preparing compounds of Formula (V), or salts thereof from the compound of Formula (X) as described or provided herein, are provided. In some embodiments, the process comprises contacting the compound of Formula (X) as described or provided herein, with a fluoride-containing reagent to produce the compound having a formula of

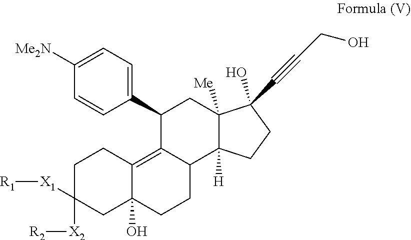

or a salt thereof. In some embodiments, the compound of Formula (X) or a salt form thereof has a formula of

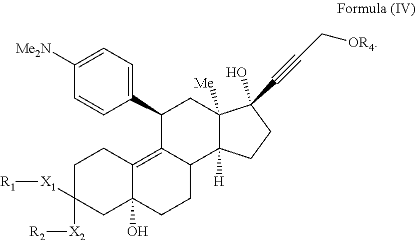

In some embodiments, methods or processes of preparing compounds of Formula (XI), or salts thereof from the compound of Formula (X) as described or provided herein, are provided. In some embodiments, the process comprises contacting the compound of Formula (X) as described or provided herein, with a fluoride-containing reagent to produce the compound having a formula of

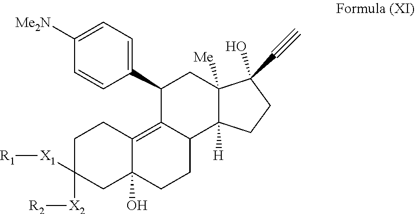

or a salt thereof.

In some embodiments, methods or processes of preparing compounds of Formula (V), or salts thereof, from contacting the compound of Formula (XI) as described or provided herein, are provided. In some embodiments, the process comprises contacting the compound of

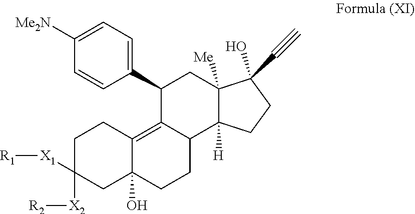

with paraformaldehyde, at least one base, and CuI under suitable conditions to produce the compound of

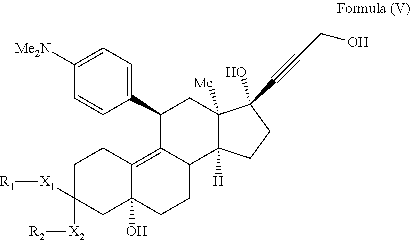

or a salt thereof.

In some embodiments, methods or processes of preparing compounds of Formula (X), or salts thereof, as described or provided herein, further comprise contacting the compound of Formula (X) in a third organic solvent with a fluoride-containing reagent to produce the compound having a formula of

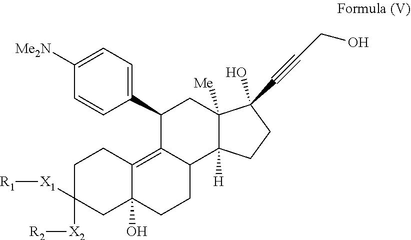

or

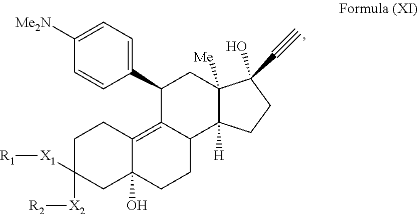

wherein R₁, R₂, X₁, and X₂ are as provided for herein and, for example, can be selected from the respective groups of chemical moieties described herein.

In some embodiments, methods or processes of preparing a compound of Formula (VII), or a pharmaceutically acceptable salt thereof are provided. In some embodiments, the process comprises:
a) subjecting the compound of

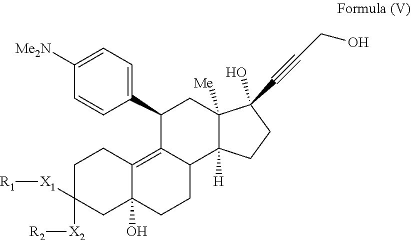

or a salt form thereof, prepared according to any process as described or provided herein under a hydrogenation condition to produce the compound of

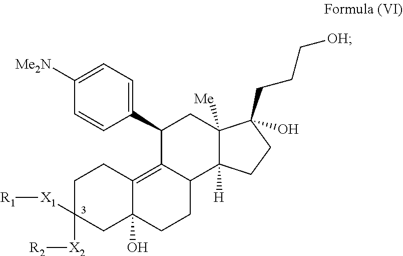

and
b) hydrolyzing the compound of Formula (VI) under a hydrolysis condition to produce the compound of

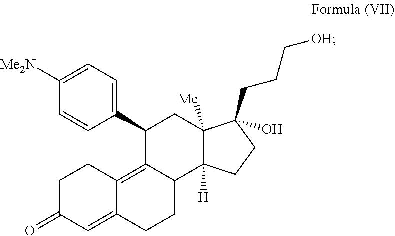 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt.

In some embodiments, methods or processes of preparing a compound of Formula (XII), or a pharmaceutically acceptable salt thereof are provided. In some embodiments, the process comprises:
subjecting the compound of

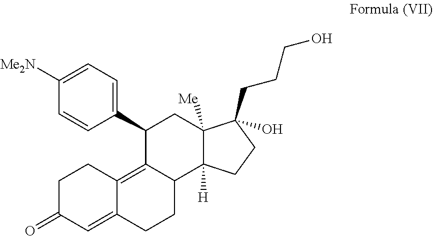 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt thereof, prepared according to any process as described or provided herein under a suitable N-demethylation condition to produce the compound of

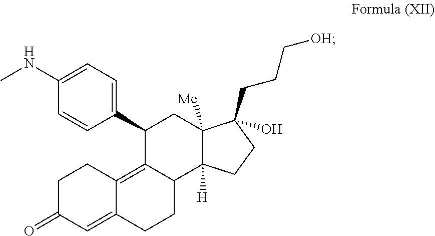 [SMILES: CNc1ccc([C@H]2C[C@]3(C)[C@@H](CC[C@]3(O)CCCO)C3CCC4=CC(=O)CCC4=C32)cc1]

or a pharmaceutically acceptable salt.

In some embodiments, also provided are compounds having a formula of

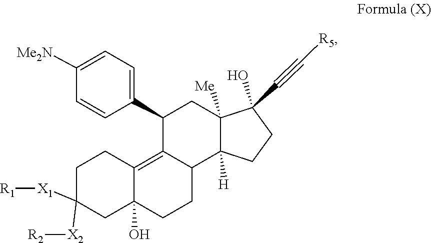

or salts thereof, wherein R₁, R₂, R₅, X₁, and X₂ are as provided for herein and, for example, can be selected from the respective groups of chemical moieties described herein. In some embodiments, also provided is a compound of Formula (X) or a salt thereof has a formula of

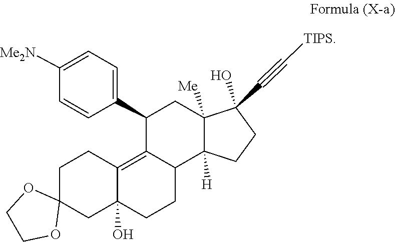

In some embodiments, also provided are compounds having a formula of

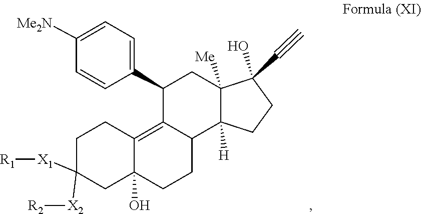

or a pharmaceutically acceptable salt thereof, wherein the variables are as defined herein. In some embodiments, the compound of Formula (XI) has a formula of

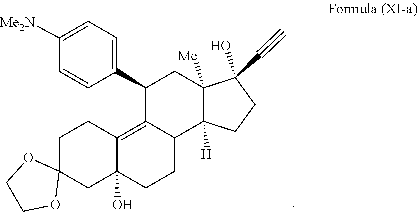 [SMILES: C#C[C@@]1(O)CC[C@H]2C3CC[C@@]4(O)CC5(CCC4=C3[C@@H](c3ccc(N(C)C)cc3)C[C@]21C)OCCO5]

In some embodiments, provided are compounds having a formula of

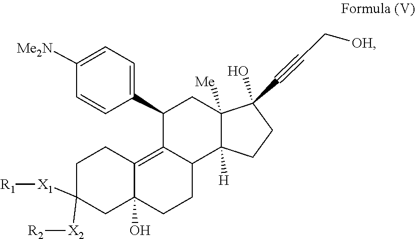

or a pharmaceutically acceptable salt thereof, wherein the variables are as defined herein. In some embodiments, the compound of Formula (V) has a formula of

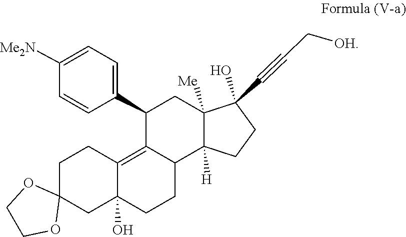 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

In some embodiments, provided are compounds having a formula of

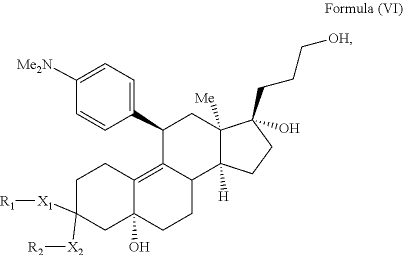

or a pharmaceutically acceptable salt thereof, wherein the variables are as defined herein. In some embodiments, the compound of Formula (VI) has a formula of

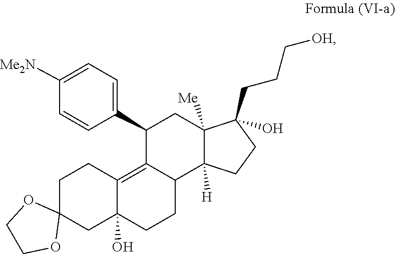 [SMILES: CN(C)c1ccc([C@H]2C[C@]3(C)[C@@H](CC[C@]3(O)CCCO)C3CC[C@@]4(O)CC5(CCC4=C32)OCCO5)cc1]

### BRIEF DESCRIPTION OF DRAWINGS

FIG. 1: X-ray powder diffraction (XRPD) pattern of the onapristone crystalline recrystallized from ethyl acetate and n-heptane in Example 7.

FIG. 2: shows a Differential Scanning calorimetry (DSC) thermogram of the onapristone crystalline recrystallized from ethyl acetate in Example 6.

FIG. 3: shows a Differential Scanning calorimetry (DSC) thermogram of the onapristone crystalline recrystallized from ethyl acetate and n-heptane in Example 7.

FIG. 4: shows the chemical formulae and their corresponding names.

### DETAILED DESCRIPTION

Unless defined otherwise, all technical and scientific terms have the same meaning as is commonly understood by one of ordinary skill in the art to which the embodiments disclosed belongs. In the event that there is a plurality of definitions for terms cited herein, those in this section prevail unless otherwise stated. All patents, applications, published applications, and other publications cited herein are incorporated by reference in their entirety.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the term “alcohol” means any organic compound in which a hydroxyl group (—OH) is bound to a carbon atom, which in turn is bound to other hydrogen and/or carbon atoms. For example, the term “alcohol” means a straight or branched alkyl-OH group of 1 to 20 carbon atoms, including, but not limited to, methanol, ethanol, n-propanol, isopropanol, t-butanol, and the like. In some embodiments, the alkyl-OH chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the terms “alkoxy” refers to an alkyl group, each optionally substituted, that is bonded through an oxygen atom. For example, the term “alkoxy” means a straight or branched-O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH2-).

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the terms “ambient temperature” and “room temperature” or “RT”, as used herein, are understood in the art and generally refer to a temperature, e.g., a reaction temperature, that is about the temperature of the room in which the reaction is carried out, for example, a temperature from about 20° C. to about 30° C., such as at or about 25° C.

As used herein, the term “amide” refers to a functional group containing a carbonyl group linked to a nitrogen atom or any compound containing the amide functional group. For example, amides are derived from carboxylic acids and amines.

As used herein, the term “aryl” means a monocyclic, bicyclic, or polycyclic (e.g., having 2, 3, or 4 fused rings) aromatic hydrocarbons. In some embodiments, aryl groups have from 6 to 20 carbon atoms or from 6 to 10 carbon atoms. Examples of aryl groups include, but are not limited to phenyl, naphthyl, anthracenyl, phenanthrenyl, indanyl, indenyl, tetrahydronaphthyl, and the like. Examples of aryl groups include, but are not limited to:

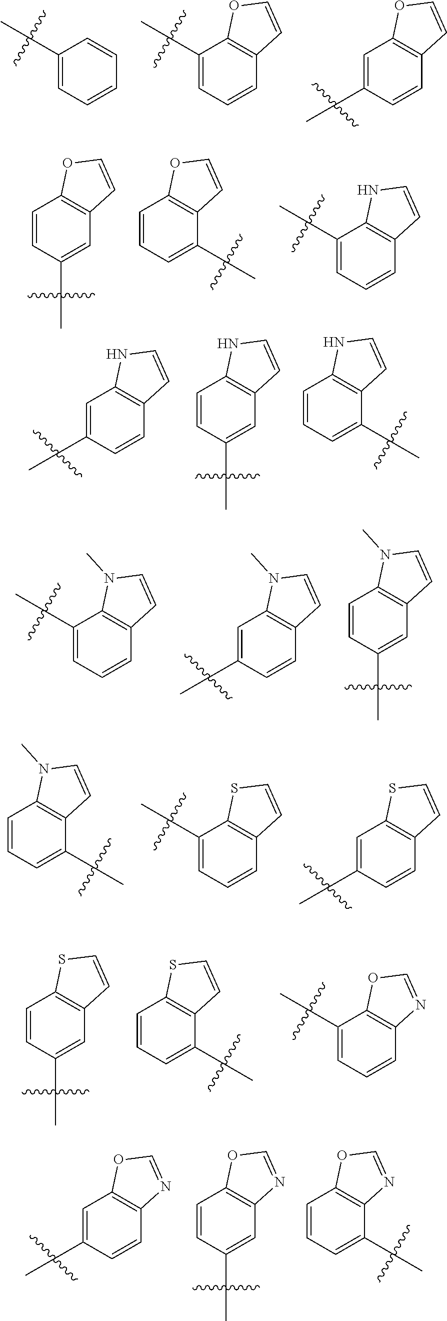

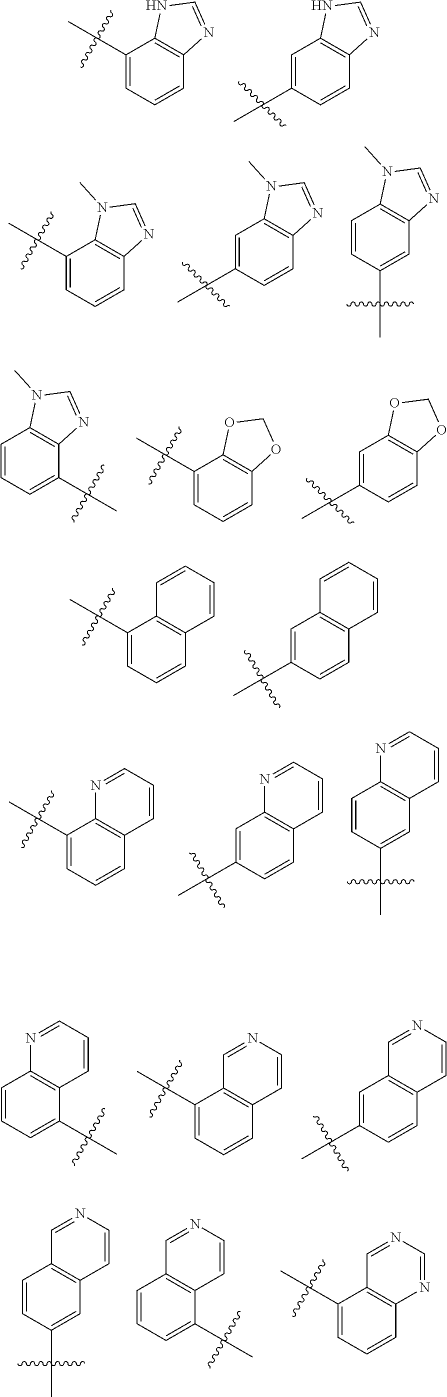

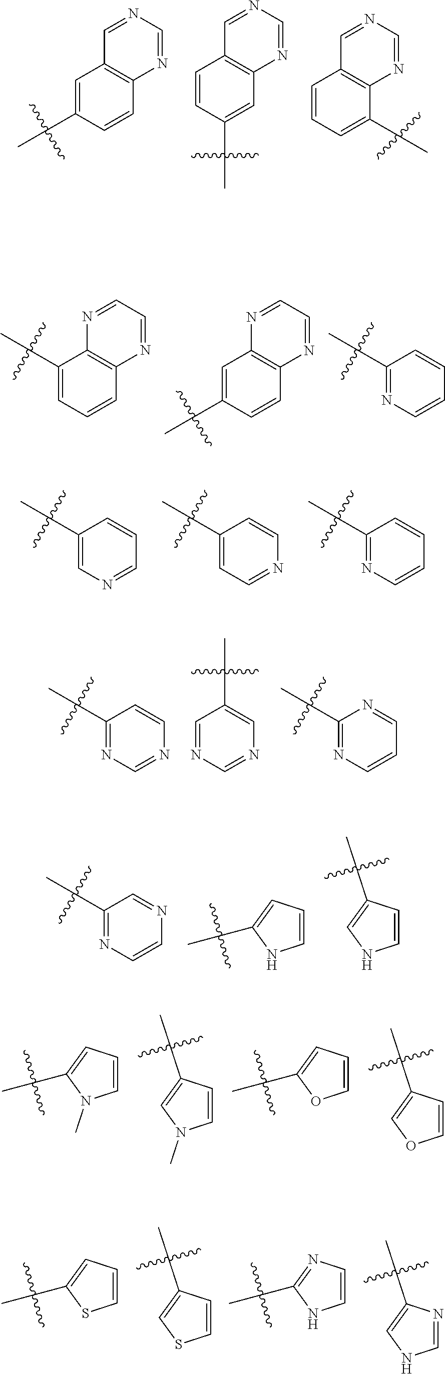

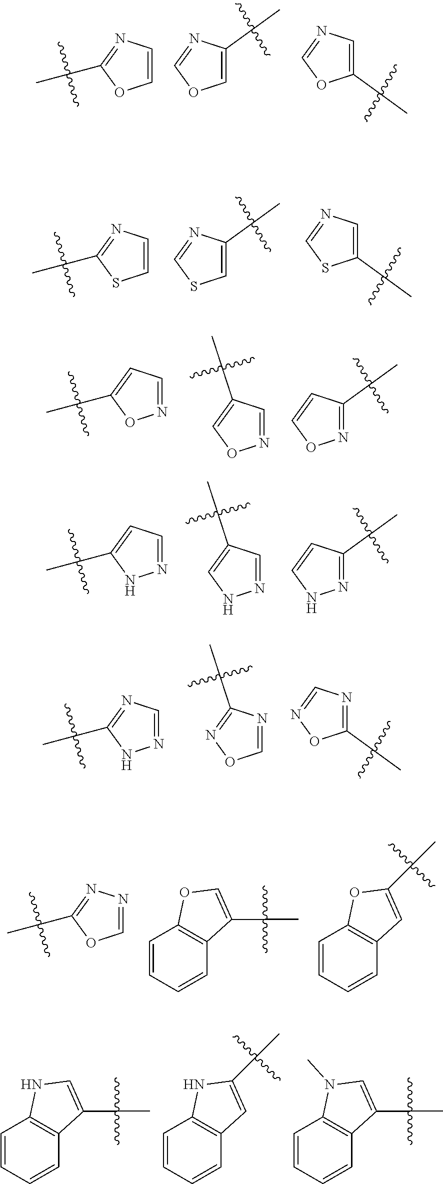

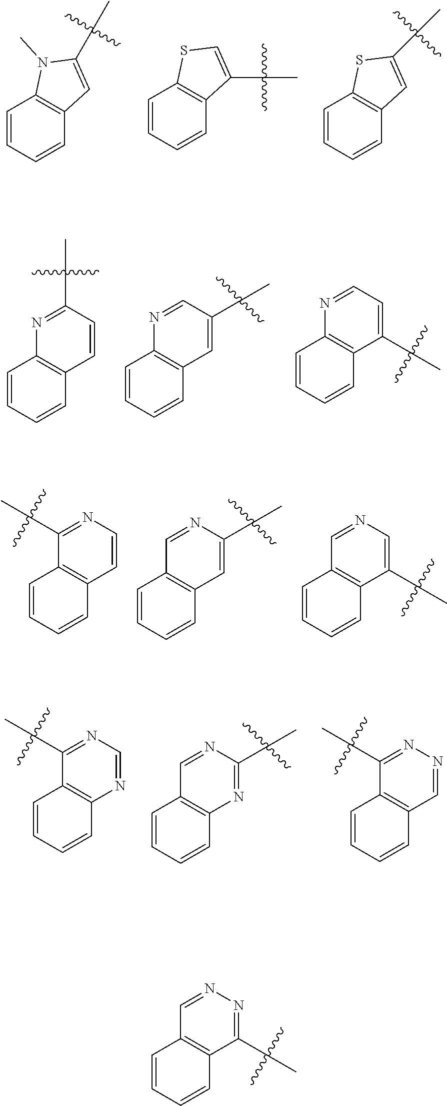

As used herein, the term “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two compounds/atoms to form at least one covalent bond between the compounds or atoms.

As used herein, the term “cyano” means-CN.

As used herein, the term “cyclic ketal” refers to a ketal in the molecule of which the ketal carbon and one or both oxygen atoms thereon are members of a ring. In some embodiments, the cyclic ketal is a 4-, 5-, 6-, 7-, or 8-membered ring.

As used herein, the term “cyclic thioketal” refers to the sulfur analog of a cyclic ketal with one of the two oxygens replaced by sulfur. In some embodiments, the cyclic thioketal is a cyclic ketal with one of the two oxygens is replaced by sulfur. In some embodiments, the cyclic thioketal is a “cyclic dithioketal,” when both oxygens of a cyclic ketal are replaced by sulfur. In some embodiments, the cyclic thioketal is a 4-, 5-, 6-, 7-, or 8-membered ring.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2 (3H)-one-1-yl).

As used herein, the term “cycloheteroalkyl” means as used herein alone or as part of another group refers to a 5-, 6- or 7-membered saturated or partially unsaturated ring which includes 1 to 2 hetero atoms such as nitrogen, oxygen and/or sulfur, linked through a carbon atom or a heteroatom, where possible, optionally via the linker (CH₂)n (where n is 0, 1, 2 or 3). The above groups may include 1 to 4 substituents such as alkyl, halo, oxo and/or any of the substituents for alkyl or aryl set out herein. In addition, any of the cycloheteroalkyl rings can be fused to a cycloalkyl, aryl, heteroaryl or cycloheteroalkyl ring.

As used herein, the terms “for example” and “such as,” and grammatical equivalences thereof.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆ alkyl group having one or more halogen substituents. Examples of haloalkyl groups include, but are not limited to, CF₃, C₂F₅, CH₂F, CHF₂, CCl₃, CHCl₂, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3, or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N. O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7 (4H)-one-5-yl, isoindolin-1-one-3-yl, and 3,4-dihydroisoquinolin-1 (2H)-one-3yl groups. Ring-forming carbon atoms and heteroatoms of the heterocycloalkyl group can be optionally substituted by oxo or sulfido.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “isolating” means that separating the compounds described herein from other components of a synthetic organic chemical reaction mixture by conventional techniques, such as filtration.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

In some embodiments, the salt of a compound described herein is a pharmaceutically acceptable salt thereof. As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments also include quaternary ammonium salts of the compounds described herein, where the compounds have one or more tertiary amine moiety.

As used herein, the term “phenyl” means-C₆H₅. A phenyl group can be unsubstituted or substituted with one, two, or three suitable substituents.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the term “solution/suspension” means a liquid composition wherein a first portion of the active agent is present in solution and a second portion of the active agent is present in particulate form, in suspension in a liquid matrix.

As used herein, the term “solvent” means a usually liquid substance capable of dissolving or dispersing one or more other substances including water, inorganic nonaqueous solvent, and organic solvents. The term “inorganic nonaqueous solvent” means a solvent other than water that is not an organic compound. Examples of the “inorganic nonaqueous solvent” include, but are not limited to: liquid ammonia, liquid sulfur dioxide, sulfuryl chloride, and sulfuryl chloride fluoride, phosphoryl chloride, dinitrogen tetroxide, antimony trichloride, bromine pentafluoride, hydrogen fluoride, pure sulfuric acid, and other inorganic acids. The term “organic solvent” means carbon-based solvent. Examples of the “organic solvent” include, but are not limited to: aromatic compounds, e.g., benzene and toluene, alcohols, e.g., methanol, ethanol, and propanol, esters and ethers, ketones, e.g., acetone, amines, nitrated and halogenated hydrocarbons. The “organic solvent” includes both polar and non-polar organic solvent. The “polar organic solvent” means an organic solvent that has large dipole moments (aka “partial charges”) and in general the organic solvent with dielectric constants greater than about 5 is considered as “polar organic solvent” while those with dielectric constants less than 5 are considered “non-polar organic solvent.” Examples of the “polar organic solvent” include, but are not limited to, acetic acid, methanol, acetone, and acetonitrile, DMSO, and DMF. Examples of the non-polar organic solvent include, but are not limited to, benzene, carbon tetrachloride, and n-hexane. The “organic solvent” includes both protonic and non-protonic organic solvents. The term “protonic organic solvent” means an organic solvent having a hydrogen atom bonded to oxygen or nitrogen (an acidic hydrogen atom). Examples of the “protonic organic solvent” include, but are not limited to, methanol, ethanol, propanol, isopropanol, butanol, hexanol, phenol, acetic acid, benzoic acid and partly fluorinated compounds thereof. Examples of the “non-protonic organic solvent” include, but are not limited to: ethylene glycol dimethyl ether, ethylene glycol methylethyl ether, diethylene glycol dimethyl ether, diethylene glycol methyl ethyl ether, triethylene glycol dimethyl ether, tetraethylene glycol dimethyl ether, ethylene glycol diethyl ether, diethylene glycol diethyl ether, 1,3-dimethoxypropane, 1,2-dimethoxypropane, propylene glycol dimethyl ether, dipropylene glycol dimethyl ether, dioxane, dimethyl carbonate, ethyl methyl carbonate, diethyl carbonate, ethylene carbonate, propylene carbonate, 2,3-dimethylethylene carbonate, butylene carbonate, acetonitrile, methoxy acetonitrile, propionitrile, butyrolactone, valerolactone, dimethoxyethane, sulforane, methylsulfolane, sulfolane, dimethyl sulfone, ethylmethyl sulfone, and isopropyl methyl sulfone.

As used herein, the phrase “substantially isolated” means a compound that is at least partially or substantially separated from the environment in which it is formed or detected.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆ alkyl, C₁-C₆ alkenyl, C₁-C₆ alkynyl, C₅-C₆ aryl, C₁-C₆ alkoxy, C₃-C₅ heteroaryl, C₃-C₆ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(C₆ aryl), —N(C₅-C₆ aryl)₂, —CHO, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

At various places in the present specification, substituents of compounds may be disclosed in groups or in ranges. It is specifically intended that embodiments include each and every individual subcombination of the members of such groups and ranges. For example, the term “C₁-C₆ alkyl” is specifically intended to individually disclose methyl, ethyl, propyl, C₄ alkyl, C₅ alkyl, and C₆ alkyl.

For compounds in which a variable appears more than once, each variable can be a different moiety selected from the Markush group defining the variable. For example, where a structure is described as having two R groups that are simultaneously present on the same compound, the two R groups can represent different moieties selected from the Markush groups defined for R. In another example, when an optionally multiple substituent is designated in the form, for example,

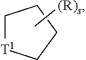

then it is understood that substituent R can occur s number of times on the ring, and R can be a different moiety at each occurrence. In the above example, where the variable T¹ is defined to include hydrogens, such as when T¹ is CH₂, NH, etc., any H can be replaced with a substituent.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompass the processes, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Processes of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are provided herein. Cis and trans geometric isomers of the compounds are also included within the present embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

In some embodiments, the composition comprises a compound, or a pharmaceutically acceptable salt thereof, that is at least 90%, at least 95%, at least 98%, or at least 99%, or 100% enantiomeric pure, which means that the ratio of one enantiomer to the other in the composition is at least 90:10, at least 95:5, at least 98:2, or at least 99:1, or is completely in the form of one enantiomer over the other.

Resolution of racemic mixtures of compounds can be carried out by any of numerous processes known in the art, including, for example, chiral HPLC, fractional recrystallization using a chiral resolving acid which is an optically active, salt-forming organic acid. Suitable resolving agents for fractional recrystallization processes include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization processes include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers, which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Processes for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of the dicarbonyl unit to adopt an anti-arrangement of the two carbonyl moiety and alter the periodicity of the compound. Pyromellitic anhydride represents still another alternative to simple amide linkages, which can alter the conformation and physical properties of the compound. Modern processes of solid-phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

Embodiments of various processes of preparing compounds of any of Formulae as described or provided herein such as Formulae (II), (III), (IV), (V), (VI), (VII), (IX), (X), (XI), (XII), (II-a), (III-a), (IV-a), (V-a), (VI-a), (X-a), (XI-a), and (XII) and salts thereof are provided. Where a variable is not specifically recited, the variable can be any option described herein, except as otherwise noted or dictated by context.

In some embodiments, the methods or processes of preparing compounds any formula of Formulae (II), (III), (IV), (V), (VI), (VII), (IX), (X), (XI), (XII), (II-a), (III-a), (IV-a), (V-a), (VI-a), (X-a), (XI-a), and (XII), or salts thereof is as described in the appended exemplary, non-limiting claims.

In some embodiments, processes of preparing compounds of Formula (II), or pharmaceutically acceptable salts thereof are provided. In some embodiments, the process comprises:
contacting the compound of

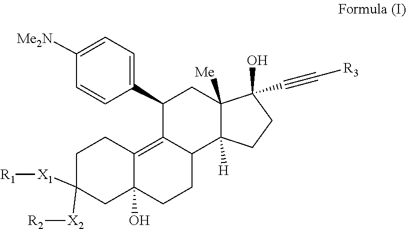

with a base in a first organic solvent under suitable conditions to produce the compound of

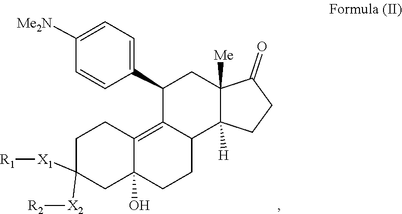

wherein:
X₁ and X₂ are each independently O or S;
R₁ and R₂ are each independently bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₃ is H, optionally substituted C₁-C₆ alkyl, optionally substituted C₁-C₆ hydroxyalkyl, optionally substituted C₁-C₆ alkoxy, optionally substituted cycloalkyl.

In some embodiments, provided are processes of preparing compounds of Formula (II), or a pharmaceutically acceptable salt thereof, wherein X₁ and X₂ are each independently O or S. In some embodiments, X₁ is O or S. In some embodiments, X₁ is O. In some embodiments, X₁ is S. In some embodiments, X₂ is O or S. In some embodiments, X₂ is O. In some embodiments, X₂ is S. In some embodiments, X₁ and X₂ are both S. In some embodiments, X₁ and X₂ are both O.

In some embodiments, provided are processes of preparing compounds of Formula (II), or a pharmaceutically acceptable salt thereof, wherein R¹ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₁ is bond. In some embodiments, R₁ is optionally substituted C₁-C₆ alkyl. In some embodiments, R₁ is optionally substituted C₁-C₆ hydroxyalkyl. In some embodiments. R₁ is optionally substituted C₁-C₆ alkoxy. In some embodiments. R₁ is optionally substituted cycloalkyl. In some embodiments, R₁ is optionally substituted cycloheteroalkyl.

In some embodiments, provided are processes of preparing compounds of Formula (II), or a pharmaceutically acceptable salt thereof, wherein R₂ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂ is a bond. In some embodiments. R₂ is optionally substituted C₁-C₆ alkyl. In some embodiments, R₂ is optionally substituted C₁-C₆ hydroxyalkyl. In some embodiments, R₂ is optionally substituted C₁-C₆ alkoxy. In some embodiments, R₂ is optionally substituted cycloalkyl. In some embodiments, R₂ is optionally substituted cycloheteroalkyl.

In some embodiments, provided are processes of preparing compounds of Formula (II), or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal or cyclic thioketal. In some embodiments. R₁, R₂, X₁, and X₂ are together with the carbon atom connected X₁ and X₂ to form optionally substituted 4-, 5-, 6-, 7-, or 8-membered cyclic thioketal.

In some embodiments, provided are processes of preparing compounds of Formula (II), or a pharmaceutically acceptable salt thereof, wherein R₁, R₂, X₁, and X₂ are together with the carbon atom connected X₁ and X₂ to form optionally substituted 5-, 6-, 7-, or 8-membered cyclic ketal. In some embodiments. R₁, R₂, X₁, and X₂ are together with the carbon atom connected X₁ and X₂ to form optionally substituted 6-, 7-, or 8-membered cyclic ketal. In some embodiments, R₁, R₂, X₁, and X₂ are together with the carbon atom connected X₁ and X₂ to form optionally substituted 7- or 8-membered cyclic ketal. In some embodiments, R₁, R₂, X₁, and X₂ are together with the carbon atom connected X₁ and X₂ to form optionally substituted 4-, 5-, 6-, or 7-membered cyclic ketal. In some embodiments, R₁, R₂, X₁, and X₂ are together with the carbon atom connected X₁ and X₂ to form optionally substituted 4-, 5-, or 6-membered cyclic ketal. In some embodiments, R₁, R₂, X₁, and X₂ are together with the carbon atom connected X₁ and X₂ to form optionally substituted 4- or 5-membered cyclic ketal. In some embodiments, R₁, R₂, X₁, and X₂ are together with the carbon atom connected X₁ and X₂ to form optionally substituted 5- or 6-membered cyclic ketal. In some embodiments, R₁, R₂, X₁, and X₂ are together with the carbon atom connected X₁ and X₂ to form optionally substituted 4-membered cyclic ketal. In some embodiments, R₁, R₂, X₁, and X₂ are together with the carbon atom connected X₁ and X₂ to form optionally substituted 5-membered cyclic ketal. In some embodiments, R₁, R₂, X₁, and X₂ are together with the carbon atom connected X₁ and X₂ to form optionally substituted 6-membered cyclic ketal. In some embodiments, R₁, R₂. X₁, and X₂ are together with the carbon atom connected X₁ and X₂ to form optionally substituted 7-membered cyclic ketal. In some embodiments, R₁, R₂, X₁, and X₂ are together with the carbon atom connected X₁ and X₂ to form optionally substituted 8-membered cyclic ketal.

In some embodiments, provided are processes of preparing compounds of Formula (II), or a pharmaceutically acceptable salt thereof, wherein R³ is H, optionally substituted C₁-C₆ alkyl, optionally substituted C₁-C₆ hydroxyalkyl, optionally substituted C₁-C₆ alkoxy, optionally substituted cycloalkyl. In some embodiments, R₃ is H. In some embodiments. R₃ is optionally substituted C₁-C₆ alkyl. In some embodiments, R₃ is Me, Et, Pr, or Bu. In some embodiments, R₃ is Me. In some embodiments, R₃ is Et. In some embodiments, R₃ is Pr. In some embodiments, R₃ is Bu. In some embodiments, R₃ is optionally substituted C₁-C₆ hydroxyalkyl. In some embodiments. R₃ is optionally substituted C₁-C₆ alkoxy. In some embodiments. R₃ is optionally substituted cycloalkyl.

In some embodiments, provided are processes of preparing compounds of Formula (II), or a pharmaceutically acceptable salt thereof, wherein R₁ and R₂ are each independently Me, Et, Pr, or Bu. In some embodiments, R₁ is Me, Et, Pr, or Bu. In some embodiments, R₁ is Me. In some embodiments, R₁ is Et. In some embodiments, R₁ is Pr. In some embodiments, R₁ is Bu. In some embodiments, R₂ is Me, Et, Pr, or Bu. In some embodiments, R₂ is Me. In some embodiments, R₂ is Et. In some embodiments, R₂ is Pr. In some embodiments, R₂ is Bu.

In some embodiments, provided are processes of preparing compounds of Formula (II), or a pharmaceutically acceptable salt thereof, wherein the compound of Formula (I) has a formula of

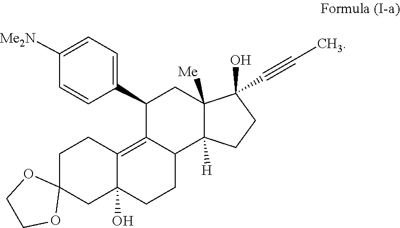 [SMILES: CC#C[C@]1(O)CC[C@H]2C3CC[C@@]4(O)CC5(CCC4=C3[C@@H](c3ccc(N(C)C)cc3)C[C@@]21C)OCCO5]

In some embodiments, provided are processes of preparing compounds of Formula (II), or a pharmaceutically acceptable salt thereof, wherein the compound of Formula (II) has a formula of

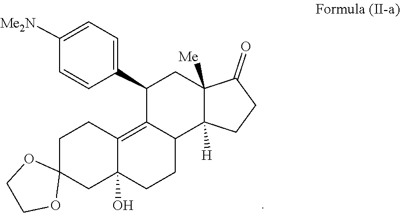 [SMILES: CN(C)c1ccc([C@H]2C[C@]3(C)C(=O)CC[C@H]3C3CC[C@@]4(O)CC5(CCC4=C32)OCCO5)cc1]

In some embodiments, provided are processes of preparing compounds of Formula (II), or a pharmaceutically acceptable salt thereof. In some embodiments, the base is an alkali metal hydroxide. In some embodiments, the base is potassium hydroxide. In some embodiments, the base is tBuOK.

In some embodiments, provided are processes of preparing compounds of Formula (II), or a pharmaceutically acceptable salt thereof, wherein the suitable condition comprises heating the mixture of the compound of Formula (I) and the base in the organic solvent to a temperature. In some embodiments, the temperature is at least about 30° C. In some embodiments, the temperature is at least about 40° C. In some embodiments. In some embodiments, the temperature is at least about 50° C. In some embodiments. In some embodiments, the temperature is at least about 60° C. In some embodiments, the temperature is at least about 70° C. In some embodiments, the temperature is at least about 80° C. In some embodiments, the temperature is between about 30° C. and about 80° C. In some embodiments, the temperature is between about 40° C. and about 80° C. In some embodiments, the temperature is between about 50° C. and about 80° C. In some embodiments, the temperature is between about 60° C. and about 80° C. In some embodiments, the temperature is between about 70° C. and about 80° C. In some embodiments, the temperature is between about 30° C. and about 90° C. In some embodiments, the temperature is between about 40° C. and about 90° C. In some embodiments, the temperature is between about 50° C. and about 90° C. In some embodiments, the temperature is between about 60° C. and about 90° C. In some embodiments, the temperature is between about 70° C. and about 90° C. In some embodiments, the temperature is about 30° C. In some embodiments, the temperature is about 40° C. In some embodiments, the temperature is about 50° C. In some embodiments, the temperature is about 60° C. In some embodiments, the temperature is about 70° C. In some embodiments, the temperature is about 80° C. In some embodiments, the temperature is about 90° C.

In some embodiments, provided are processes of preparing compounds of Formula (II), or a pharmaceutically acceptable salt thereof, wherein the heated mixture is stirred at the temperature for at least about 5 minutes. In some embodiments, the heated mixture is stirred at the temperature for at least about 10 minutes. In some embodiments, the heated mixture is stirred at the temperature for at least about 20 minutes. In some embodiments, the heated mixture is stirred at the temperature for at least about 30 minutes. In some embodiments, the heated mixture is stirred at the temperature for at least about 60 minutes. In some embodiments, the heated mixture is stirred at the temperature for at least about 2 hours. In some embodiments, the heated mixture is stirred at the temperature for at least about 8 hours. In some embodiments, the heated mixture is stirred at the temperature for about 5 minutes. In some embodiments, the heated mixture is stirred at the temperature for about 10 minutes. In some embodiments, the heated mixture is stirred at the temperature for about 20 minutes. In some embodiments, the heated mixture is stirred at the temperature for about 30 minutes. In some embodiments, the heated mixture is stirred at the temperature for about 60 minutes. In some embodiments, the heated mixture is stirred at the temperature for about 2 hours. In some embodiments, the heated mixture is stirred at the temperature for about 8 hours.

In some embodiments, provided are processes of preparing compounds of Formula (II), or a pharmaceutically acceptable salt thereof, wherein further comprising cooling the heated mixture to room temperature. In some embodiments, the heated mixture was cooled to room temperature over 5 minutes. In some embodiments, the heated mixture was cooled to room temperature over 10 minutes. In some embodiments, the heated mixture was cooled to room temperature over 30 minutes. In some embodiments, the heated mixture was cooled to room temperature over 60 minutes. In some embodiments, the heated mixture was cooled to room temperature over 2 hours. In some embodiments, the heated mixture was cooled to room temperature over 4 hours. In some embodiments, the heated mixture was cooled to room temperature over 8 hours.

In some embodiments, provided are processes of preparing compounds of Formula (II), or a pharmaceutically acceptable salt thereof, further comprising adding NH₄Cl aqueous solution to the resulting mixture to form a suspension. In some embodiments, the process further comprises filtering the suspension to yield a solid. In some embodiments, the process further comprises crystallizing the solid to produce the compound of Formula (II). In some embodiments, the solid is crystallized in a solvent. In some embodiments, the solvent is an alcohol, H₂O, an ether, an alkane, an ester, a ketone, or a combination thereof. In some embodiments, the solvent is an alcohol. In some embodiments, the alcohol is methanol, ethanol, propanol, isopropanol, butanol, or a combination thereof. In some embodiments, the alcohol is methanol. In some embodiments, the alcohol is ethanol. In some embodiments, the alcohol is propanol, isopropanol. In some embodiments, the alcohol is butanol. In some embodiments, the alcohol is a combination of two or more of methanol, ethanol, propanol, isopropanol, and butanol. In some embodiments, the alcohol is a combination of two of methanol, ethanol, propanol, isopropanol, and butanol. In some embodiments, the solvent is H₂O. In some embodiments, the solvent is an ether. In some embodiments, the ether is diethyl ether, diisopropyl ether, cyclopentyl methyl ether, methyl tert-butyl ether, or a combination thereof. In some embodiments, the ether is diethyl ether. In some embodiments, the ether is diisopropyl ether. In some embodiments, the ether is cyclopentyl methyl ether. In some embodiments, the ether is methyl tert-butyl ether. In some embodiments, the ether is a combination of two or more of diethyl ether, diisopropyl ether, cyclopentyl methyl ether and methyl tert-butyl ether. In some embodiments, the ether is a combination of two of diethyl ether, diisopropyl ether, cyclopentyl methyl ether and methyl tert-butyl ether. In some embodiments, the solvent is an alkane. In some embodiments, the alkane is butane, pentane, hexane, heptane, octane, or a combination thereof. In some embodiments, the alkane is butane. In some embodiments, the alkane is pentane. In some embodiments, the alkane is hexane. In some embodiments, the alkane is heptane. In some embodiments, the alkane is octane. In some embodiments, the alkane is a combination of two or more of butane, pentane, hexane, heptane and octane. In some embodiments, the solvent is an ester. In some embodiments, the solvent is a ketone. In some embodiments, the ketone is acetone. In some embodiments, the ketone is methyl ethyl ketone. In some embodiments, the solvent is a combination of two or more of an alcohol, H₂O, an ether, an alkane, an ester, a ketone. In some embodiments, the solvent is a combination of two of an alcohol, H₂O, an ether, an alkane, an ester, a ketone. In some embodiments, the solvent is a combination of an alcohol and an ether as described or provided herein. In some embodiments, the volume ratio of the alcohol to the ether is about 1:1. In some embodiments, the solvent is a combination of an alcohol and water as described or provided herein. In some embodiments, the volume ratio of the alcohol to the water is about 3:1.

In some embodiments, provided are processes of preparing compounds of Formula (II), or a pharmaceutically acceptable salt thereof, wherein the first organic solvent is a polar organic solvent. In some embodiments, the polar organic solvent is dimethylformamide, diethylformamide, 1-butanol, 2-butanol, iso-butanol, tert-butanol, or a combination thereof. In some embodiments, the polar organic solvent is dimethylformamide. In some embodiments, the polar organic solvent is diethylformamide. In some embodiments, the polar organic solvent is 1-butanol. In some embodiments, the polar organic solvent is 2-butanol. In some embodiments, the polar organic solvent is iso-butanol. In some embodiments, the polar organic solvent is tert-butanol. In some embodiments, the polar organic solvent is a combination of two or more of dimethylformamide, diethylformamide, 1-butanol, 2-butanol, iso-butanol, tert-butanol. In some embodiments, the polar organic solvent is a combination of two of dimethylformamide, diethylformamide, 1-butanol, 2-butanol, iso-butanol, tert-butanol.

In some embodiments, provided are processes of preparing compounds of Formula (II), or a pharmaceutically acceptable salt thereof, wherein the first organic solvent is a non-polar organic solvent. In some embodiments, the non-polar organic solvent is toluene, xylene, benzene, cyclohexane, methyl cyclohexane, hexane, heptane, 1,4-dioxane, or a combination thereof. In some embodiments, the polar organic solvent is toluene. In some embodiments, the polar organic solvent is xylene. In some embodiments, the polar organic solvent is benzene. In some embodiments, the polar organic solvent is cyclohexane. In some embodiments, the polar organic solvent is methyl cyclohexane. In some embodiments, the polar organic solvent is hexane. In some embodiments, the polar organic solvent is heptane. In some embodiments, the polar organic solvent is a combination of two or more of toluene, xylene, benzene, cyclohexane, methyl cyclohexane, hexane, heptane, 1,4-dioxane. In some embodiments, the non-polar organic solvent is a combination of two of toluene, xylene, benzene, cyclohexane, methyl cyclohexane, hexane, heptane, 1,4-dioxane.

In some embodiments, processes of preparing compounds of Formula (II), or a pharmaceutically acceptable salt thereof, as described or provided herein, further comprise adding water to the cooled mixture and extracting the resulting aqueous phase with a second organic solvent to produce the compound of Formula (II). In some embodiments, the second organic solvent is a polar organic solvent. In some embodiments, the polar organic solvent is a non-protic polar organic solvent. In some embodiments, the non-protic polar organic solvent is ethyl acetate.

In some embodiments, methods or processes of preparing a compound of Formula (III) or a pharmaceutically acceptable salt thereof are provided. In some embodiments, the process comprises:
irradiating a solution of the compound of

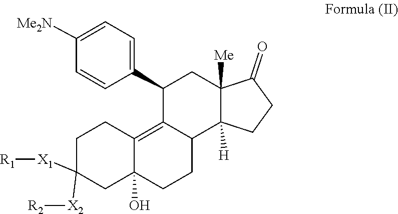

in an organic solvent with a UV light in a flow reactor to produce the compound of

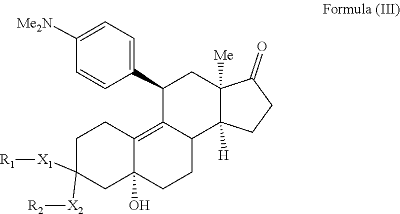

wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal.

In some embodiments, provided are processes of preparing compounds of Formula (III), or a pharmaceutically acceptable salt thereof, wherein X₁ and X₂ are each independently O or S. In some embodiments, X₁ is O or S. In some embodiments, X₁ is O. In some embodiments, X₁ is S. In some embodiments, X₂ is O or S. In some embodiments, X₂ is O. In some embodiments, X₂ is S. In some embodiments, X₁ and X₂ are both S. In some embodiments, X₁ and X₂ are both O.

In some embodiments, provided are processes of preparing compounds of Formula (III), or a pharmaceutically acceptable salt thereof, wherein R¹ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₁ is a bond. In some embodiments. R₁ is optionally substituted C₁-C₆ alkyl. In some embodiments, R₁ is optionally substituted C₁-C₆ hydroxyalkyl. In some embodiments. R₁ is optionally substituted C₁-C₆ alkoxy. In some embodiments, R₁ is optionally substituted cycloalkyl. In some embodiments, R₁ is optionally substituted cycloheteroalkyl.

In some embodiments, provided are processes of preparing compounds of Formula (III), or a pharmaceutically acceptable salt thereof, wherein R₂ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂ is a bond. In some embodiments, R₂ is optionally substituted C₁-C₆ alkyl. In some embodiments, R₂ is optionally substituted C₁-C₆ hydroxyalkyl. In some embodiments. R₂ is optionally substituted C₁-C₆ alkoxy. In some embodiments, R₂ is optionally substituted cycloalkyl. In some embodiments, R₂ is optionally substituted cycloheteroalkyl.

In some embodiments, provided are processes of preparing compounds of Formula (III), or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal or cyclic thioketal. In some embodiments, R₁, R₂, X₁, and X₂ are together with the carbon atom connected X₁ and X₂ to form optionally substituted 4-, 5-, 6-, 7-, or 8-membered cyclic thioketal.

In some embodiments, provided are processes of preparing compounds of Formula (III), or a pharmaceutically acceptable salt thereof, wherein R₁, R₂, X₁, and X₂ are together with the carbon atom connected X₁ and X₂ to form optionally substituted 5-, 6-, 7-, or 8-membered cyclic ketal. In some embodiments. R₁, R₂, X₁, and X₂ are together with the carbon atom connected X₁ and X₂ to form optionally substituted 6-, 7-, or 8-membered cyclic ketal. In some embodiments, R₁, R₂, X₁, and X₂ are together with the carbon atom connected X₁ and X₂ to form optionally substituted 7- or 8-membered cyclic ketal. In some embodiments, R₁, R₂, X₁, and X₂ are together with the carbon atom connected X₁ and X₂ to form optionally substituted 4-, 5-, 6-, or 7-membered cyclic ketal. In some embodiments, R₁, R₂, X₁, and X₂ are together with the carbon atom connected X₁ and X₂ to form optionally substituted 4-, 5-, or 6-membered cyclic ketal. In some embodiments, R₁, R₂, X₁, and X₂ are together with the carbon atom connected X₁ and X₂ to form optionally substituted 4- or 5-membered cyclic ketal. In some embodiments, R₁, R₂, X₁, and X₂ are together with the carbon atom connected X₁ and X₂ to form optionally substituted 5- or 6-membered cyclic ketal. In some embodiments, R₁, R₂, X₁, and X₂ are together with the carbon atom connected X₁ and X₂ to form optionally substituted 4-membered cyclic ketal. In some embodiments, R₁, R₂, X₁, and X₂ are together with the carbon atom connected X₁ and X₂ to form optionally substituted 5-membered cyclic ketal. In some embodiments. R₁, R₂, X₁, and X₂ are together with the carbon atom connected X₁ and X₂ to form optionally substituted 6-membered cyclic ketal. In some embodiments, R₁, R₂, X₁, and X₂ are together with the carbon atom connected X₁ and X₂ to form optionally substituted 7-membered cyclic ketal. In some embodiments, R₁, R₂, X₁, and X₂ are together with the carbon atom connected X₁ and X₂ to form optionally substituted 8-membered cyclic ketal.

In some embodiments, provided are processes of preparing compounds of Formula (III), or a pharmaceutically acceptable salt thereof, wherein R¹ and R₂ are each independently Me, Et, Pr, or Bu. In some embodiments. R₁ is Me, Et, Pr, or Bu. In some embodiments, R₁ is Me. In some embodiments, R₁ is Et. In some embodiments, R₁ is Pr. In some embodiments, R₁ is Bu. In some embodiments. R₂ is Me, Et, Pr, or Bu. In some embodiments, R₂ is Me. In some embodiments, R₂ is Et. In some embodiments, R₂ is Pr. In some embodiments, R₂ is Bu.

In some embodiments, provided are processes of preparing a compound of Formula (III), or a pharmaceutically acceptable salt thereof, wherein the compound of Formula (II) has a formula of

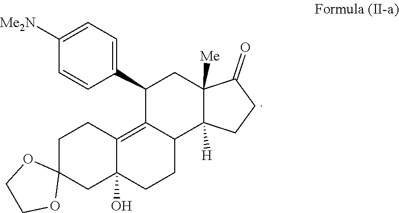 [SMILES: CN(C)c1ccc([C@H]2C[C@]3(C)C(=O)CC[C@H]3C3CC[C@@]4(O)CC5(CCC4=C32)OCCO5)cc1]

In some embodiments, provided are processes of preparing a compound of Formula (III), or a pharmaceutically acceptable salt thereof, wherein the compound of Formula (III) has a formula of

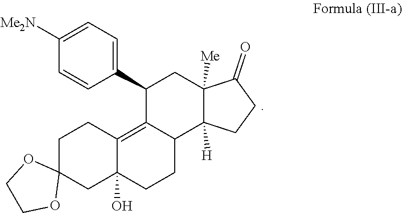 [SMILES: CN(C)c1ccc([C@H]2C[C@@]3(C)C(=O)CC[C@H]3C3CC[C@@]4(O)CC5(CCC4=C32)OCCO5)cc1]

In some embodiments, provided are processes of preparing a compound of Formula (III), or a pharmaceutically acceptable salt thereof, wherein the UV light is a narrow band frequency light at a wavelength from about 300 nm to about 320 nm. In some embodiments, the UV light is a narrow band frequency light with a wavelength at about 311 nm. In some embodiments, the UV light is from a low-pressure mercury lamp. In some embodiments, the UVB light is from a fluorescent bulb. In some embodiments, the low-pressure mercury lamp is fitted inside the flow reactor.

In some embodiments, provided are processes of preparing a compound of Formula (III), or a pharmaceutically acceptable salt thereof, wherein the flow reactor is fabricated from a long polytetrafluoroethylene (PTFE) tubing with an inner diameter between 1 to 20 millimeter. In some embodiments, the flow reactor is fabricated from quartz.

In some embodiments, provided are processes of preparing a compound of Formula (III), or a pharmaceutically acceptable salt thereof, wherein the flow reactor is fabricated from a long fluorinated ethylene propylene (FEP) tubing with an inner diameter between 1 to 20 millimeter. In some embodiments, the flow reactor is fabricated from quartz.

In some embodiments, provided are processes of preparing a compound of Formula (III), or a pharmaceutically acceptable salt thereof, wherein the flow reactor is fabricated from a long perfluoroalkoxy alkane (PFA) tubing with an inner diameter between 1 to 20 millimeter. In some embodiments, the flow reactor is fabricated from quartz.

In some embodiments, provided are processes of preparing a compound of Formula (III), or a pharmaceutically acceptable salt thereof, wherein the organic solvent is ethyl acetate, toluene, methyltetrahydrofuran, tetrahydrofuran, isopropyl ether, methanol, dioxane, dichloromethane, isopropylether or a combination thereof. In some embodiments. In some embodiments, the organic solvent is ethyl acetate. In some embodiments, the organic solvent is toluene. In some embodiments, the organic solvent is methyltetrahydrofuran. In some embodiments, the organic solvent is tetrahydrofuran. In some embodiments, the organic solvent is isopropyl ether. In some embodiments, the organic solvent is methanol. In some embodiments, the organic solvent is dioxane. In some embodiments, the organic solvent is dichloromethane. In some embodiments, the organic solvent is isopropylether a combination of two or more of ethyl acetate, toluene, methyltetrahydrofuran, tetrahydrofuran, isopropyl ether, methanol, dioxane, dichloromethane, and isopropylether.

In some embodiments, processes of preparing a compound of Formula (III), or a pharmaceutically acceptable salt thereof, as described or provided herein, further comprises washing the solution with aqueous NaHSO₃ solution to remove the aldehyde impurities from the UV irradiation.

In some embodiments, processes of preparing a compound of Formula (III), or a pharmaceutically acceptable salt thereof as described or provided herein, further comprise purifying the crude compound of Formula (III) with solid absorbents such as a silica gel, magnesium silicate, alumina, polymers, clays or other porous or high surface area solids. In some embodiments, the solid absorbent is silica gel. In some embodiments, the solid absorbent is magnesium silicate. In some embodiments, the solid absorbent is alumina. In some embodiments, the solid absorbent is a polymer. In some embodiments, the solid absorbent is clay. In some embodiments, the solid absorbent is a porous or high surface area solid.

In some embodiments, processes of preparing a compound of Formula (III), or a pharmaceutically acceptable salt thereof, as described or provided herein, further comprise recrystallizing the compound of Formula (III). In some embodiments, the compound of Formula (III) is recrystallized from a combination of organic solvents. In some embodiments, the compound of Formula (III) is recrystallized from ethyl acetate and heptane. In some embodiments, the volume ratio of ethyl acetate to heptane is about 1 to 3. In some embodiments, the volume ratio of ethyl acetate to heptane is from about 20 to 1 to about 1 to 20. In some embodiments, the volume ratio of ethyl acetate to heptane is from about 10 to 1 to about 1 to 10. In some embodiments, the volume ratio of ethyl acetate to heptane is from about 1 to 1 to about 1 to 20. In some embodiments, the volume ratio of ethyl acetate to heptane is from about 1 to 1 to about 1 to 10. In some embodiments, the volume ratio of ethyl acetate to heptane is about 1 to 1. In some embodiments, the volume ratio of ethyl acetate to heptane is about 1 to 2. In some embodiments, the volume ratio of ethyl acetate to heptane is about 1 to 3. In some embodiments, the volume ratio of ethyl acetate to heptane is about 1 to 4. In some embodiments, the volume ratio of ethyl acetate to heptane is about 1 to 5. In some embodiments, the volume ratio of ethyl acetate to heptane is about 1 to 6. In some embodiments, the volume ratio of ethyl acetate to heptane is about 1 to 7. In some embodiments, the volume ratio of ethyl acetate to heptane is about 1 to 8. In some embodiments, the volume ratio of ethyl acetate to heptane is about 1 to 9. In some embodiments, the volume ratio of ethyl acetate to heptane is about 1 to 10.

In some embodiments, methods or processes of preparing a compound of Formula (X), or a salt thereof, are provided. In some embodiments, the process comprises: contacting the compound of

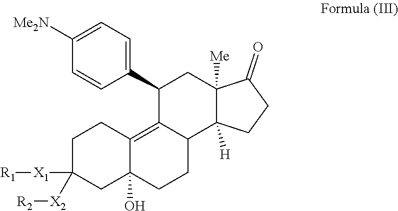

with an organometallic reagent having a formula of

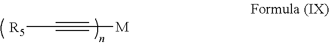

to produce the compound of

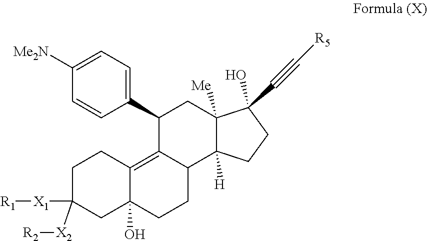

or a salt form thereof; and
wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₅ is trialkylsilyl or triakylsilyloxy.

In some embodiments, provided are processes of preparing compounds of Formula (X), or a pharmaceutically acceptable salt thereof, wherein X₁ and X₂ are each independently O or S. In some embodiments, X₁ is O or S. In some embodiments, X₁ is O. In some embodiments, X₁ is S. In some embodiments, X₂ is O or S. In some embodiments, X₂ is O. In some embodiments, X₂ is S. In some embodiments, X₁ and X₂ are both S. In some embodiments, X₁ and X₂ are both O.

In some embodiments, provided are processes of preparing compounds of Formula (X), or a pharmaceutically acceptable salt thereof, wherein R¹ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₁ is a bond. In some embodiments, R₁ is optionally substituted C₁-C₆ alkyl. In some embodiments, R₁ is optionally substituted C₁-C₆ hydroxyalkyl. In some embodiments, R₁ is optionally substituted C₁-C₆ alkoxy. In some embodiments, R₁ is optionally substituted cycloalkyl. In some embodiments, R₁ is optionally substituted cycloheteroalkyl.

In some embodiments, provided are processes of preparing compounds of Formula (X), or a pharmaceutically acceptable salt thereof, wherein R₂ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂ is a bond. In some embodiments, R₂ is optionally substituted C₁-C₆ alkyl. In some embodiments, R₂ is optionally substituted C₁-C₆ hydroxyalkyl. In some embodiments, R₂ is optionally substituted C₁-C₆ alkoxy. In some embodiments, R₂ is optionally substituted cycloalkyl. In some embodiments, R₂ is optionally substituted cycloheteroalkyl.

In some embodiments, provided are processes of preparing compounds of Formula (X), or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal or cyclic thioketal. In some embodiments. R₁, R₂, X₁, and X₂ are together with the carbon atom connected X₁ and X₂ to form optionally substituted 4-, 5-, 6-, 7-, or 8-membered cyclic thioketal.

In some embodiments, provided are processes of preparing compounds of Formula (X), or a pharmaceutically acceptable salt thereof, wherein R₁, R₂, X₁, and X₂ are together with the carbon atom connected X₁ and X₂ to form optionally substituted 5-, 6-, 7-, or 8-membered cyclic ketal. In some embodiments, R₁, R₂, X₁, and X₂ are together with the carbon atom connected X₁ and X₂ to form optionally substituted 6-, 7-, or 8-membered cyclic ketal. In some embodiments, R₁, R₂, X₁, and X₂ are together with the carbon atom connected X₁ and X₂ to form optionally substituted 7- or 8-membered cyclic ketal. In some embodiments, R₁, R₂, X₁, and X₂ are together with the carbon atom connected X₁ and X₂ to form optionally substituted 4-, 5-, 6-, or 7-membered cyclic ketal. In some embodiments, R₁, R₂, X₁, and X₂ are together with the carbon atom connected X₁ and X₂ to form optionally substituted 4-, 5-, or 6-membered cyclic ketal. In some embodiments, R₁, R₂, X₁, and X₂ are together with the carbon atom connected X₁ and X₂ to form optionally substituted 4- or 5-membered cyclic ketal. In some embodiments, R₁, R₂, X₁, and X₂ are together with the carbon atom connected X₁ and X₂ to form optionally substituted 5- or 6-membered cyclic ketal. In some embodiments, R₁, R₂, X₁, and X₂ are together with the carbon atom connected X₁ and X₂ to form optionally substituted 4-membered cyclic ketal. In some embodiments, R₁, R₂, X₁, and X₂ are together with the carbon atom connected X₁ and X₂ to form optionally substituted 5-membered cyclic ketal. In some embodiments, R₁, R₂, X₁, and X₂ are together with the carbon atom connected X₁ and X₂ to form optionally substituted 6-membered cyclic ketal. In some embodiments, R₁, R₂. X₁, and X₂ are together with the carbon atom connected X₁ and X₂ to form optionally substituted 7-membered cyclic ketal. In some embodiments, R₁, R₂, X₁, and X₂ are together with the carbon atom connected X₁ and X₂ to form optionally substituted 8-membered cyclic ketal.

In some embodiments, provided are processes of preparing compounds of Formula (X), or a pharmaceutically acceptable salt thereof, wherein R₁ and R₂ are each independently Me, Et, Pr, or Bu. In some embodiments, R₁ is Me, Et, Pr, or Bu. In some embodiments, R₁ is Me. In some embodiments, R₁ is Et. In some embodiments. R₁ is Pr. In some embodiments, R₁ is Bu. In some embodiments, R₂ is Me, Et, Pr, or Bu. In some embodiments, R₂ is Me. In some embodiments, R₂ is Et. In some embodiments, R₂ is Pr. In some embodiments, R₂ is Bu.

In some embodiments, provided are processes of preparing compounds of Formula (X), or a pharmaceutically acceptable salt thereof, wherein M is Li, Na, K. MgBr, CuBr, CuLi, Mg, Cu, or Al. In some embodiments, M is Li. In some embodiments, M is Na. In some embodiments, M is K. In some embodiments, M is MgBr. In some embodiments, M is CuBr. In some embodiments, M is CuLi. In some embodiments, M is Mg. In some embodiments, M is Cu. In some embodiments, M is Al.

In some embodiments, provided are processes of preparing compounds of Formula (X), or a pharmaceutically acceptable salt thereof, wherein n is 1-3. In some embodiments, n is 1-2. In some embodiments, n is 1-2. In some embodiments, n is 2-3. In some embodiments, n is 1. In some embodiments, n is 2. In some embodiments, n is 3.

In some embodiments, provided are processes of preparing compounds of Formula (X), or a pharmaceutically acceptable salt thereof, wherein R₅ is trialkylsilyl or triakylsilyloxy. In some embodiments, R₅ is trialkylsilyl. In some embodiments, R₅ is triakylsilyloxy. In some embodiments, the trialkylsilyl is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS). In some embodiments, the trialkylsilyloxy is trimethylsilyloxy (OTMS), tricthylsilyloxy (OTES), tert-butyldimethylsilyloxy (OTBS), tert-butyldiphenylsilyloxy (OTBDPS), thexyldimethylsilyloxy, or triisopropylsilyloxy (OTIPS).

In some embodiments, provided are processes of preparing compounds of Formula (X), or a pharmaceutically acceptable salt thereof, wherein the organometallic reagent of Formula VIII is nucleophilic. In some embodiments, the organometallic reagent of Formula VIII is an organolithium reagent. In some embodiments, the organolithium reagent is R₅≡Li. In some embodiments, the organolithium reagent is commercially available.

In some embodiments, methods or processes of preparing the organolithium reagent of Formula (IX) are provided. In some embodiments, the process comprises contacting R₅≡H with an alkyllithium reagent in a first organic solvent at a low temperature. In some embodiments, the alkyllithium reagent is n-butyllithum, n-heptanyllithoum or n-hexyllithium, or a combination thereof. In some embodiments, the alkyllithium reagent is n-butyllithum. In some embodiments, the alkyllithium reagent is n-heptanyllithoum. In some embodiments, the alkyllithium reagent is n-hexyllithium.

In some embodiments, provided are processes of preparing the organolithium reagent of Formula (IX), wherein the low temperature is below −10° C. In some embodiments, the low temperature is below about −20° C. In some embodiments, the low temperature is below about-30° C. In some embodiments, the low temperature is below about −40° C. In some embodiments, the low temperature is below about −50° C. In some embodiments, the low temperature is below about −60° C. In some embodiments, the low temperature is below about −70° C. In some embodiments, the low temperature is about −10° C. In some embodiments, the low temperature is about −20° C. In some embodiments, the low temperature is about −30° C. In some embodiments, the low temperature is about −40° C. In some embodiments, the low temperature is about −50° C. In some embodiments, the low temperature is about −60° C. In some embodiments, the low temperature is about −70° C. In some embodiments, the low temperature is about −80° C. In some embodiments, the compound of Formula (III) or a salt form thereof is separately dissolved in a second organic solvent before contacting the organolithium reagent at the low temperature. In some embodiments, the compound of Formula (III) or a salt form thereof in the second organic solvent is added dropwise to the organolithium reagent in the first organic solvent at the low temperature.

In some embodiments, provided are processes of preparing compounds of Formula (IX), or a pharmaceutically acceptable salt thereof, wherein both the first and second organic solvents are polar non-protic organic solvents. In some embodiments, both the first and second organic solvents are tetrahydrofuran or 2-methyltetrohydrafuran. In some embodiments, both the first and second organic solvents are tetrahydrofuran. In some embodiments, both the first and second organic solvents are 2-methyltetrohydrafuran.

In some embodiments, provided are processes of preparing compounds of Formula (X), or a pharmaceutically acceptable salt thereof, wherein the molar ratio of the organolithium reagent to the compound of Formula (III) is between about 1:1 to about 10:1. In some embodiments, the molar ratio is between about 1:1 to about 9:1. In some embodiments, the molar ratio is between about 1:1 to about 8:1. In some embodiments, the molar ratio is between about 1:1 to about 7:1. In some embodiments, the molar ratio is between about 1:1 to about 6:1. In some embodiments, the molar ratio is between about 1:1 to about 5:1. In some embodiments, the molar ratio is between about 1:1 to about 4:1. In some embodiments, the molar ratio is between about 1:1 to about 3:1. In some embodiments, the molar ratio is between about 1:1 to about 2:1. In some embodiments, the molar ratio is about 1:1. In some embodiments, the molar ratio is about 2:1. In some embodiments, the molar ratio is about 3:1. In some embodiments, the molar ratio is about 4:1. In some embodiments, the molar ratio is about 5:1. In some embodiments, the molar ratio is about 6:1. In some embodiments, the molar ratio is about 7:1. In some embodiments, the molar ratio is about 8:1. In some embodiments, the molar ratio is about 9:1. In some embodiments, the molar ratio is about 10:1.

In some embodiments, provided are processes of preparing compounds of Formula (X), or a pharmaceutically acceptable salt thereof, wherein a diastereofacial selectivity for the compound of Formula (X) to the 17-β-hydroxy-epimer thereof is achieved. In some embodiments, the molar ratio of the compound of Formula (X) to the 17-β-hydroxy-epimer thereof is from about 1:1 to about 20:1, from about 2:1 to about 20:1, from about 3:1 to about 20:1, from about 4:1 to about 20:1, from about 5:1 to about 20:1, from about 6:1 to about 20:1, from about 7:1 to about 20:1, from about 8:1 to about 20:1, from about 9:1 to about 20:1, from about 10:1 to about 20:1, about 11:1 to about 20:1, from about 12:1 to about 20:1, from about 13:1 to about 20:1, from about 14:1 to about 20:1, from about 15:1 to about 20:1, from about 16:1 to about 20:1, from about 17:1 to about 20:1, from about 18:1 to about 20:1, or from about 19:1 to about 20:1. In some embodiments, the diastereofacial selectivity is more than about 1:1. In some embodiments, the diastereofacial selectivity is more than about 2:1. In some embodiments, the diastereofacial selectivity is more than about 3:1. In some embodiments, the diastereofacial selectivity is more than about 4:1. In some embodiments, the diastereofacial selectivity is more than about 5:1. In some embodiments, the diastereofacial selectivity is more than about 6:1. In some embodiments, the diastereofacial selectivity is more than about 7:1. In some embodiments, the diastereofacial selectivity is more than about 8:1. In some embodiments, the diastereofacial selectivity is more than about 9:1. In some embodiments, the diastereofacial selectivity is more than about 10:1. In some embodiments, the diastereofacial selectivity is more than about 11:1. In some embodiments, the diastereofacial selectivity is more than about 12:1. In some embodiments, the diastereofacial selectivity is more than about 13:1. In some embodiments, the diastereofacial selectivity is more than about 14:1. In some embodiments, the diastereofacial selectivity is more than about 15:1. In some embodiments, the diastereofacial selectivity is more than about 16:1. In some embodiments, the diastereofacial selectivity is more than about 17:1. In some embodiments, the diastereofacial selectivity is more than about 18:1. In some embodiments, the diastereofacial selectivity is more than about 19:1. In some embodiments, the diastereofacial selectivity is more than about 20:1. In some embodiments, the diastereofacial selectivity is about 1:1. In some embodiments, the diastereofacial selectivity is about 2:1. In some embodiments, the diastereofacial selectivity is about 3:1. In some embodiments, the diastereofacial selectivity is about 4:1. In some embodiments, the diastereofacial selectivity is about 5:1. In some embodiments, the diastereofacial selectivity is about 6:1. In some embodiments, the diastereofacial selectivity is about 7:1. In some embodiments, the diastereofacial selectivity is about 8:1. In some embodiments, the diastereofacial selectivity is about 9:1. In some embodiments, the diastereofacial selectivity is about 10:1. In some embodiments, the diastereofacial selectivity is about 11:1. In some embodiments, the diastereofacial selectivity is about 12:1. In some embodiments, the diastereofacial selectivity is about 13:1. In some embodiments, the diastereofacial selectivity is about 14:1. In some embodiments, the diastereofacial selectivity is about 15:1. In some embodiments, the diastereofacial selectivity is about 16:1. In some embodiments, the diastereofacial selectivity is about 17:1. In some embodiments, the diastereofacial selectivity is about 18:1. In some embodiments, the diastereofacial selectivity is about 19:1. In some embodiments, the diastereofacial selectivity is about 20:1.

In some embodiments, processes of preparing compounds of Formula (IX), or a pharmaceutically acceptable salt thereof, as described or provided herein, further comprise isolating of the compound of Formula (X). In some embodiments, the isolating of the compound of Formula (X) comprises: quenching the resulting mixture with ice water to form an aqueous phase and an organic phase; optionally washing the organic phase with water and brine; optionally drying the organic phase with a drying reagent; and evaporating the organic phase to form the compound of Formula (X). In some embodiments, the drying reagent is anhydrous Na₂SO₄, MgSO₄, or CaSO₄, or a combination thereof. In some embodiments, the drying reagent is anhydrous Na₂SO.

In some embodiments, processes of preparing compounds of Formula (X), or salts thereof, as described or provided herein, further comprise contacting the compound of Formula (X) in a third organic solvent with a fluoride-containing reagent to produce the compound having a formula of

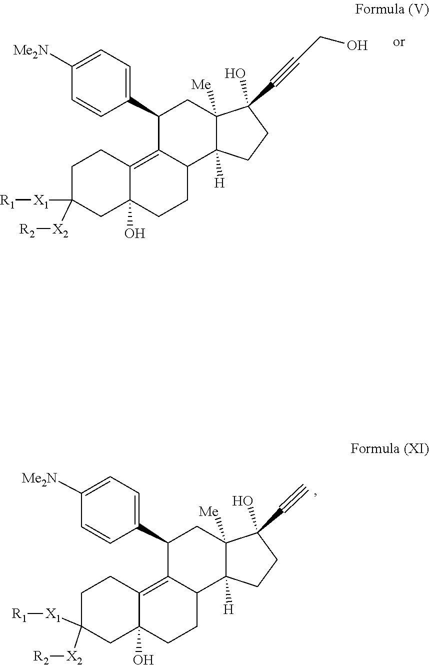

wherein the variables are as described and defined herein. In some embodiments, the fluoride-containing reagent is tetra-n-butylammonium fluoride (TBAF), NH₄F, KH₂F, KF, hydrogen fluoride, hydrogen fluoride pyridine, tris(dimethylamino)sulfonium difluorotrimethylsilicate, triethylamine trihydrofluoride (Et₃N·3HF), tetrabutylammonium tetra(t-butyl alcohol)-coordinated fluoride (TBAF(tBuOH)₄), tetrabutylammonium triphenyldifluorosilicate, or a combination thereof. In some embodiments, the fluoride-containing reagent is tetra-n-butylammonium fluoride (TBAF). In some embodiments, the fluoride-containing reagent is NH₄F. In some embodiments, the fluoride-containing reagent is KH₂F. In some embodiments, the fluoride-containing reagent is KF. In some embodiments, the fluoride-containing reagent is hydrogen fluoride. In some embodiments, the fluoride-containing reagent is hydrogen fluoride pyridine. In some embodiments, the fluoride-containing reagent is tris(dimethylamino)sulfonium difluorotrimethylsilicate. In some embodiments, the fluoride-containing reagent is triethylamine trihydrofluoride (Et₃N·3HF). In some embodiments, the fluoride-containing reagent is tetrabutylammonium tetra(t-butyl alcohol)-coordinated fluoride (TBAF(tBuOH)₄). In some embodiments, the fluoride-containing reagent is tetrabutylammonium triphenyldifluorosilicate. In some embodiments, the fluoride-containing reagent is a combination of two or more of tetra-n-butylammonium fluoride (TBAF), NH₄F, KH₂F, KF, hydrogen fluoride, hydrogen fluoride pyridine, tris(dimethylamino)sulfonium difluorotrimethylsilicate, triethylamine trihydrofluoride (Et₃N·3HF), tetrabutylammonium tetra(t-butyl alcohol)-coordinated fluoride (TBAF(tBuOH)₄), and tetrabutylammonium triphenyldifluorosilicate. In some embodiments, the fluoride-containing reagent is tetra-n-butylammonium fluoride (TBAF), NH₄F, KH₂F, KF, or a combination thereof. In some embodiments, the fluoride-containing reagent is a combination of two or more of tetra-n-butylammonium fluoride (TBAF), NH₄F, KH₂F, and KF.

In some embodiments, the fluoride-containing reagent is added to the solution of the compound of Formula (X) in the third organic solvent to form a mixture. In some embodiments, the mixture was optionally heated to a temperature and kept at the temperature for a period of time. In some embodiments, the temperature is at room temperature. In some embodiments, the temperature is at least about 40° C. In some embodiments, the temperature is at least about 50° C. In some embodiments, the temperature is at least about 60° C. In some embodiments, the temperature is at least about 70° C. In some embodiments, the temperature is at least about 80° C. In some embodiments, the period of time is at least about 5 minutes. In some embodiments, the period of time is at least about 10 minutes. In some embodiments, the period of time is at least about 20 minutes. In some embodiments, the period of time is at least about 30 minutes. In some embodiments, the period of time is at least about 60 minutes. In some embodiments, the period of time is at least about 2 hours. In some embodiments, the period of time is at least about 4 hours. In some embodiments, the period of time is about 12 hours. In some embodiments, the period of time is about 16 hours. In some embodiments, the period of time is about 20 hours. In some embodiments, the third organic solvent is a polar non-protic organic solvent. In some embodiments, the third organic solvent is dimethyl sulfoxide.

In some embodiments, the fluoride-containing reagent is added to the solution of the compound of Formula (X) in the third organic solvent to form a mixture. In some embodiments, the mixture was optionally cooled to a temperature and kept at the temperature for a period of time. In some embodiments, the temperature is at room temperature. In some embodiments, the temperature is at least about 0° C. In some embodiments, the temperature is at least about 10° C. In some embodiments, the temperature is at least about 20° C. In some embodiments, the period of time is at least about 5 minutes. In some embodiments, the period of time is at least about 10 minutes. In some embodiments, the period of time is at least about 20 minutes. In some embodiments, the period of time is at least about 30 minutes. In some embodiments, the period of time is at least about 60 minutes. In some embodiments, the period of time is at least about 2 hours. In some embodiments, the period of time is at least about 4 hours. In some embodiments, the period of time is about 12 hours. In some embodiments, the period of time is about 16 hours. In some embodiments, the period of time is about 20 hours. In some embodiments, the third organic solvent is a polar non-protic organic solvent. In some embodiments, the third organic solvent is 2-methyltetrahydrofuran.

In some embodiments, the heated mixture is cooled to room temperature to form a lower phase and an upper phase. In some embodiments, the lower phase is optionally washed with a non-polar organic solvent. In some embodiments, the non-polar organic solvent is one or more C₅-C₁₀ alkanes. In some embodiments, the non-polar organic solvent is pentane, hexane, heptane, or a combination thereof. In some embodiments, the non-polar organic solvent is pentane. In some embodiments, the non-polar organic solvent is hexane. In some embodiments, the non-polar organic solvent is heptane. In some embodiments, the non-polar organic solvent is a combination of two or more of pentane, hexane, and heptane. In some embodiments, the lower phase is mixed with water to form a mixture and the mixture is extracted with a polar non-protic organic solvent. In some embodiments, the mixture is extracted with the polar non-protic organic solvent at least once. In some embodiments, the mixture is extracted with the polar non-protic organic solvent twice. In some embodiments, the mixture is extracted with the polar non-protic organic solvent at least three times. In some embodiments, the mixture is extracted with the polar non-protic organic solvent at least four times.

In some embodiments, the polar non-protic organic solvent after extraction is optionally washed with water and brine, optionally dried over a drying reagent, and evaporated to form the compound having a formula of Formula (V) or Formula (XI) with the 17-β-hydroxy-epimer thereof as an impurity.

In some embodiments, the polar non-protic organic solvent after extraction is optionally washed with water and brine, optionally dried over a drying reagent, and evaporated to form the compound having a compound of Formula (V) with the 17-β-hydroxy-epimer thereof as an impurity. In some embodiments, the molar ratio of the compound of Formula (V) to the 17-β-hydroxy-epimer thereof is from about 1:1 to about 10:1, from about 2:1 to about 10:1, from about 3:1 to about 10:1, from about 4:1 to about 10:1, from about 5:1 to about 10:1, from about 6:1 to about 10:1, from about 7:1 to about 10:1, from about 8:1 to about 10:1, or from about 9:1 to about 10:1. In some embodiments, the molar ratio of the compound of Formula (V) to the 17-β-hydroxy-epimer thereof is about 2:1. In some embodiments, the molar ratio is about 3:1. In some embodiments, the molar ratio is about 4:1. In some embodiments, the molar ratio is about 5:1. In some embodiments, the molar ratio is about 6:1. In some embodiments, the molar ratio is about 7:1. In some embodiments, the molar ratio is about 8:1. In some embodiments, the molar ratio is about 9:1. In some embodiments, the molar ratio is about 10:1.

In some embodiments, the polar non-protic organic solvent after extraction is optionally washed with water and brine, optionally dried over a drying reagent, and evaporated to form the compound having a compound of Formula (V) with the 17-β-hydroxy-epimer thereof as an impurity. In some embodiments, the molar ratio of the compound of Formula (V) to the 17-β-hydroxy-epimer thereof is from about 1:1 to about 20:1, from about 2:1 to about 20:1, from about 3:1 to about 20:1, from about 4:1 to about 20:1, from about 5:1 to about 20:1, from about 6:1 to about 20:1, from about 7:1 to about 20:1, from about 8:1 to about 20:1, from about 9:1 to about 20:1, from about 10:1 to about 20:1, from about 11:1 to about 20:1, from about 12:1 to about 20:1, from about 13:1 to about 20:1, from about 14:1 to about 20:1, from about 15:1 to about 20:1, from about 16:1 to about 20:1, from about 17:1 to about 20:1, from about 18:1 to about 20:1, from about 19:1 to about 20:1. In some embodiments, the molar ratio of the compound of Formula (V) to the 17-β-hydroxy-epimer thereof is about 2:1. In some embodiments, the molar ratio is about 3:1. In some embodiments, the molar ratio is about 4:1. In some embodiments, the molar ratio is about 5:1. In some embodiments, the molar ratio is about 6:1. In some embodiments, the molar ratio is about 7:1. In some embodiments, the molar ratio is about 8:1. In some embodiments, the molar ratio is about 9:1. In some embodiments, the molar ratio is about 10:1.

In some embodiments, the polar non-protic organic solvent after extraction is optionally washed with water and brine, optionally dried over a drying reagent, and evaporated to form the compound having of Formula (X). In some embodiments, the molar ratio of the compound of Formula (XI) to the 17-β-hydroxy-epimer thereof is about 2:1. In some embodiments, the molar ratio is about 3:1. In some embodiments, the molar ratio is about 4:1. In some embodiments, the molar ratio is about 5:1. In some embodiments, the molar ratio is about 6:1. In some embodiments, the molar ratio is about 7:1. In some embodiments, the molar ratio is about 8:1. In some embodiments, the molar ratio is about 9:1. In some embodiments, the molar ratio is about 10:1. In some embodiments, the molar ratio is about 11:1. In some embodiments, the molar ratio is about 12:1. In some embodiments, the molar ratio is about 13:1. In some embodiments, the molar ratio is about 14:1. In some embodiments, the molar ratio is about 15:1. In some embodiments, the molar ratio of the compound of Formula (XI) and the 17-β-hydroxy-epimer thereof is about 15.2:1. In some embodiments, the molar ratio is about 16:1. In some embodiments, the molar ratio is about 17:1. In some embodiments, the molar ratio is about 18:1. In some embodiments, the molar ratio is about 19:1. In some embodiments, the molar ratio is about 20:1.

In some embodiments, processes of preparing compounds of Formula XI, or salts thereof, as described or provided herein, the reaction mixture was added to water to minimize decomposition. In some embodiments, Formula XI crystallized in the aqueous solvent mixture.

In some embodiments, processes of preparing compounds of Formula (X), or salts thereof, as described or provided herein, wherein R₅ is trialkylsilyl. In some embodiments, R₅ is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS). In some embodiments, the trialkylsilyl is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS).

In some embodiments, provided are processes of preparing compounds of Formula (V), or a pharmaceutically acceptable salt thereof, comprising contacting the compound of

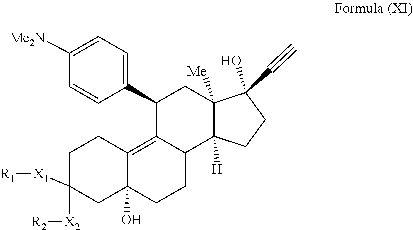

with paraformaldehyde, at least one bases, and CuI in a fourth organic solvent under suitable conditions to produce the compound of

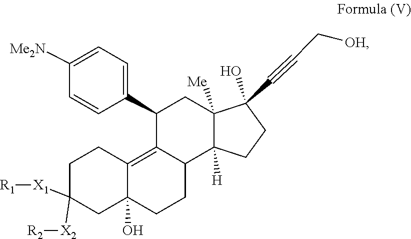

wherein the variables are as described and defined herein.

In some embodiments, processes of preparing compounds of Formula (V), or a pharmaceutically acceptable salt thereof, as described or provided herein, further comprise two bases. In some embodiments, the two bases are a trialkylamine and an alkali hydroxide. In some embodiments, the trialkylamine is triethylamine. In some embodiments, the alkali hydroxide is KOH. In some embodiments, the two bases are a trialkylamine and potassium oxide.

In some embodiments, provided are processes of preparing compounds of Formula (V), or a pharmaceutically acceptable salt thereof, wherein the fourth organic solvent is a non-protic organic solvent. In some embodiments, the non-protic organic solvent is a polar non-protic organic solvent. In some embodiments, the polar non-protic organic solvent is dimethyl sulfoxide.

In some embodiments, provided are processes of preparing compounds of Formula (V), or a pharmaceutically acceptable salt thereof, wherein the diastereofacial selectivity of the compound of Formula (V) to the 17-β-hydroxy-epimer thereof is at least about 10:1. In some embodiments, the diastereofacial selectivity is about 2:1. In some embodiments, the diastereofacial selectivity is about 3:1. In some embodiments, the diastereofacial selectivity is about 4:1. In some embodiments, the diastereofacial selectivity is about 5:1. In some embodiments, the diastereofacial selectivity is about 6:1. In some embodiments, the diastereofacial selectivity is about 7:1. In some embodiments, the diastereofacial selectivity is about 8:1. In some embodiments, the diastereofacial selectivity is about 9:1. In some embodiments, the diastereofacial selectivity is about 10:1. In some embodiments, the diastereofacial selectivity is about 11:1. In some embodiments, the diastereofacial selectivity is about 12:1. In some embodiments, the diastereofacial selectivity is about 13:1. In some embodiments, the diastereofacial selectivity is about 14:1. In some embodiments, the diastereofacial selectivity is about 15:1. In some embodiments, the diastereofacial selectivity is about 15.3:1. In some embodiments, the diastereofacial selectivity is about 16:1. In some embodiments, the diastereofacial selectivity is about 17:1. In some embodiments, the diastereofacial selectivity is about 18:1. In some embodiments, the diastereofacial selectivity is about 19:1. In some embodiments, the diastereofacial selectivity is about 20:1.

In some embodiments, provided are processes of preparing compounds of Formula (X) or salts thereof, as described or provided herein, wherein the compound of Formula (III) has a formula of

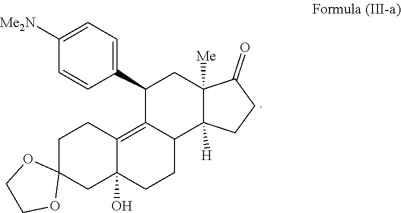 [SMILES: CN(C)c1ccc([C@H]2C[C@@]3(C)C(=O)CC[C@H]3C3CC[C@@]4(O)CC5(CCC4=C32)OCCO5)cc1]

In some embodiments, The process of claim 135, wherein the compound of Formula (X) has a formula of

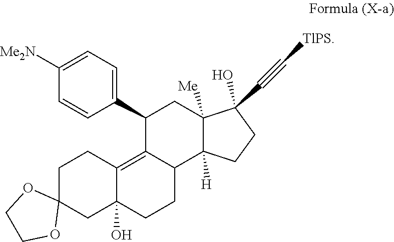

In some embodiments, provided are processes of preparing compounds of Formula (V), or salts thereof, as described or provided herein, wherein the compound of Formula (III) has a formula of

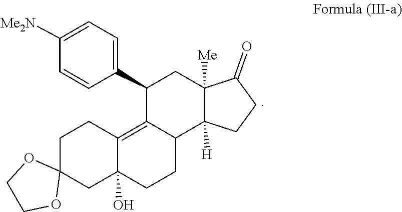 [SMILES: CN(C)c1ccc([C@H]2C[C@@]3(C)C(=O)CC[C@H]3C3CC[C@@]4(O)CC5(CCC4=C32)OCCO5)cc1]

In some embodiments, the compound of Formula (X) has a formula of

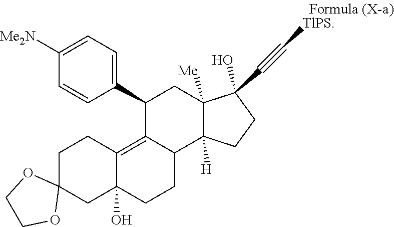

In some embodiments, provided are processes of preparing compounds of Formula (XI) or salts thereof, as described or provided herein, wherein the compound of Formula (III) has a formula of

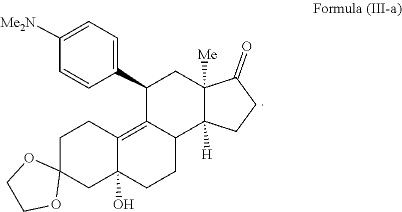 [SMILES: CN(C)c1ccc([C@H]2C[C@@]3(C)C(=O)CC[C@H]3C3CC[C@@]4(O)CC5(CCC4=C32)OCCO5)cc1]

In some embodiments, the compound of Formula (XI) has a formula of

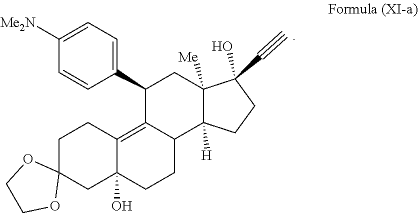 [SMILES: C#C[C@@]1(O)CC[C@H]2C3CC[C@@]4(O)CC5(CCC4=C3[C@@H](c3ccc(N(C)C)cc3)C[C@]21C)OCCO5]

In some embodiments, provided are processes of preparing compounds of Formula (X) or salts thereof, as described or provided herein, wherein the compound of Formula (X) or a salt form thereof, has a formula of

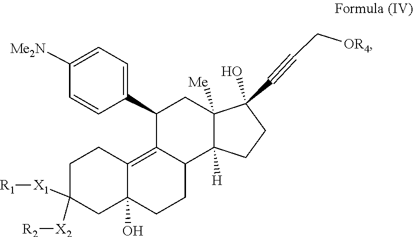

wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and R₄ is trialkylsilyl.

In some embodiments, R₄ is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS). In some embodiments, R₄ is trimethylsilyl (TMS). In some embodiments, R₄ is triethylsilyl (TES). In some embodiments, R₄ is tert-butyldimethylsilyl (TBS). In some embodiments, R₄ is tert-butyldiphenylsilyl (TBDPS). In some embodiments, R₄ is thexyldimethylsilyl. In some embodiments, R₄ is triisopropylsilyl (TIPS).

In some embodiments, the compound of Formula (III) has a formula of

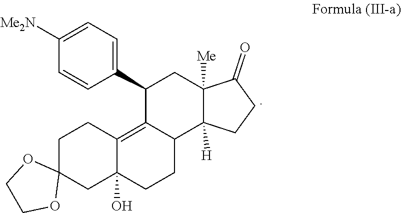 [SMILES: CN(C)c1ccc([C@H]2C[C@@]3(C)C(=O)CC[C@H]3C3CC[C@@]4(O)CC5(CCC4=C32)OCCO5)cc1]

In some embodiments, the compound of Formula (IV) has a formula of

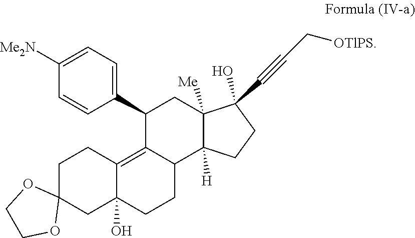

In some embodiments, provided are processes of preparing compounds of Formula (V), or salts thereof, wherein the compound of Formula (V) has a formula of

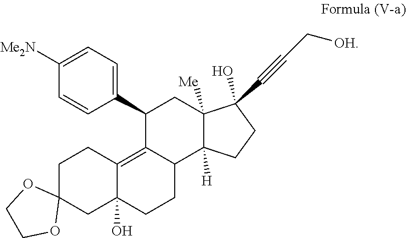 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

In some embodiments, processes of preparing compounds of Formula (V), or salts thereof, as described or provided herein, further comprise purifying the compound of Formula (V) from the 17-β-hydroxy-epimer thereof comprising forming an aggregate of the compound of Formula (V) with a salt. In some embodiments, the salt is a citrate salt or an acetate salt. In some embodiments, the salt is a citrate salt. In some embodiments, the citrate salt is lithium citrate. In some embodiments, the salt is an acetate salt. In some embodiments, the acetate salt is potassium acetate.

In some embodiments, the molar ratio of the compound of Formula (V) to the 17-β-hydroxy-epimer thereof in the aggregate is more than about 20:1, about 40:1, about 60:1, about 80:1, about 100:1, about 150:1. about 200:1, about 250:1, about 300:1, about 350:1, or about 400:1. In some embodiments, the molar ratio is more than about 20:1. In some embodiments, the molar ratio is more than about 40:1. In some embodiments, the molar ratio is more than about 60:1. In some embodiments, the molar ratio is more than about 80:1. In some embodiments, the molar ratio is more than about 100:1. In some embodiments, the molar ratio is more than about 150:1. In some embodiments, the molar ratio is more than about 200:1. In some embodiments, the molar ratio is more than about 250:1. In some embodiments, the molar ratio is more than about 300:1. In some embodiments, the molar ratio is more than about 350:1. In some embodiments, the molar ratio is more than about 400:1.

In some embodiments, the aggregate is formed in a mixture of the polar non-protic organic solvent and the non-polar organic solvent. In some embodiments, the volume ratio of the polar non-protic organic solvent to the non-polar organic solvent in the mixture is about 1:1. In some embodiments, the volume ratio is about 2:1. In some embodiments, the volume ratio is about 3:1. In some embodiments, the volume ratio is about 4:1. In some embodiments, the volume ratio is about 4.4:1. In some embodiments, the volume ratio is about 5:1. In some embodiments, the volume ratio is about 6:1. In some embodiments, the volume ratio is about 7:1. In some embodiments, the volume ratio is about 8:1. In some embodiments, the polar non-protic organic solvent is ethyl acetate. In some embodiments, the non-polar organic solvent is heptane.

In some embodiments, processes of preparing compounds of Formula (V), or salts thereof, further comprising dissolving the aggregate in the polar non-protic organic solvent to form an organic phase, washing the organic phase with water, and concentrating the organic phase to form the compound of Formula (V) in high purify. In some embodiments, the purity of the compound of Formula (V) is at least about 90%, at least about 92%, at least about 94%, at least about 96%, at least about 98%, or at least about 99%. In some embodiments, the purity is at least about 90%. In some embodiments, the purity is at least about 92%. In some embodiments, the purity is at least about 94%. In some embodiments, the purity is at least about 96%. In some embodiments, the purity is at least about 98%. In some embodiments, the purity is at least about 99%.

In some embodiments, provided are processes of preparing a compound of Formula (VII), or a pharmaceutically acceptable salt thereof, comprising: a) subjecting the compound of

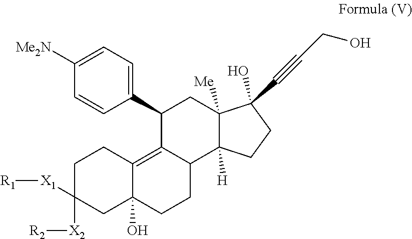

or a salt form thereof, prepared according to any process as described or provided herein under a hydrogenation condition to produce the compound of and

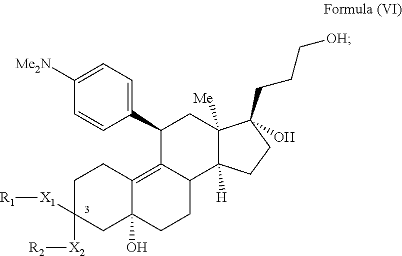

e) hydrolyzing the compound of Formula (VI) under a hydrolysis condition to produce the compound of

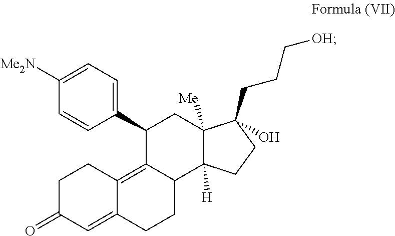 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt

In some embodiments, provided are processes of preparing a compound of Formula (VII), or a pharmaceutically acceptable salt thereof, wherein the hydrogenation condition is suitable for converting an unsaturated carbon-carbon triple bond to a saturated carbon-carbon bond. In some embodiments, the suitable hydrogenation condition comprising hydrogen and a metal catalyst. In some embodiments, the suitable hydrogenation condition comprising hydrogen, a metal catalyst, and an alkali metal borohydride. In some embodiments, wherein the alkali metal borohydride comprises sodium borohydride or potassium borohydride.

In some embodiments, provided are processes of preparing a compound of Formula (VII), or a pharmaceutically acceptable salt thereof, wherein the metal catalyst comprises palladium.

In some embodiments, provided are processes of preparing a compound of Formula (VII), or a pharmaceutically acceptable salt thereof, wherein the hydrolysis condition is a suitable condition for removing the protection for the 3-ketone of Formula (VI). In some embodiments, wherein the suitable hydrolysis condition comprises sulfuric acid. In some embodiments, wherein the suitable hydrolysis condition comprises an alkali metal bisulfate. In some embodiments, wherein the suitable hydrolysis condition comprises potassium bisulfate.

In some embodiments, processes of preparing a compound of Formula (VII), or a pharmaceutically acceptable salt thereof, as described or provided herein, further comprise recrystallizing the compound of Formula (VII). In some embodiments, the compound of Formula (VII) is recrystallized from a mixture of at least one polar solvent and/or at least non-polar solvent. In some embodiments, the compound of Formula (VII) is recrystallized from a mixture of at least one polar solvent and at least one non-polar solvent. In some embodiments, the compound of Formula (VII) is recrystallized from a mixture of one polar solvent and one non-polar solvent. In some embodiments, the polar solvent is ethyl acetate. In some embodiments, the polar solvent is n-heptane. In some embodiments, the mixture is a mixture of ethyl acetate and n-heptane. In some embodiments, the volume ratio of ethyl acetate to n-heptane is about 1 to 3.

In some embodiments, provided are processes of preparing a compound of Formula (XII), acceptable salt thereof, the process comprising: or a pharmaceutically subjecting the compound of

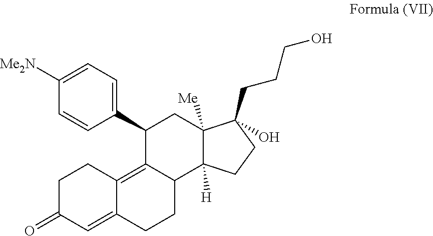 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt thereof, prepared according to any process as described or provided herein under a suitable N-demethylation condition to produce the compound of

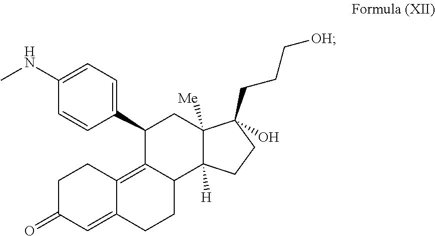 [SMILES: CNc1ccc([C@H]2C[C@]3(C)[C@@H](CC[C@]3(O)CCCO)C3CCC4=CC(=O)CCC4=C32)cc1]

or a pharmaceutically acceptable salt. In some embodiments, the N-demethylation condition is suitable for removing a methyl group from the dimethylamino phenyl group on the compound of Formula (VII). In some embodiments, the compound of Formula (XII) can be prepared from the compound of Formula (VII) under the N-demethylation protocols, including but not limited to the von Braun reaction employing cyanogen bromide (Von Braun, J. Chem. Ber. 1980, 33, 1438), using chloroformate reagents (Cooley, J. H.; Evain, E. J. Synthesis 1989, 1; Olofson, R. A. et al. J. Org. Chem. 1984, 49, 2081), photochemical methods (Ripper, J. A., et al. Biorg. & Med. Chem. Lett. 2001, 11, 443-445), demethylation of N-oxides (the Polonovski reaction as described in Kok, G. et al. Adv. Synth. Catal. 2009, 351, 283; Dong, Z. et al. J. Org. Chem. 2007, 72, 9881; Smith, C. et al. PCT Patent Application Publication No. WO 2005/028483), as well as microbial methods as described in Madyashtha, K. M. et al. Proc. Indian Acad. Sci. 1984, 106, 1203; Madyastha, K. M. et al. J. Chem. Soc. Perkin Trans. 1, 1994, 911 and enzymatic methods as described in Chaudhary, V. et al. Collect. Czech. Chem. Commun. 2009, 74, 1179), each of the references recited herein is incorporated by reference in its entirety. In some embodiments, the suitable N-demethylation condition is a von Braun reaction, a method of applying chloroformate, a Polonovski reaction, a photochemical method, a microbial method, or an enzymatic method. In some embodiments, the enzymatic method is a cytochrome P450 enzyme-mediated N-demethylation. In some embodiments, the enzymatic method is a cytochrome P450 3A-mediated N-demethylation.

In some embodiments, the methods or processes of preparing compounds of any formula of Formulae (II), (III), (IV), (V), (VI), (VII), (IX), (X), (XI), (XII), (II-a), (III-a), (IV-a), (V-a), (VI-a), (X-a), (XI-a), and (XII), or salts thereof or pharmaceutically acceptable salts thereof, are as described in the appended exemplary, non-limiting claims.

In some embodiments, also provided are compounds having a formula of

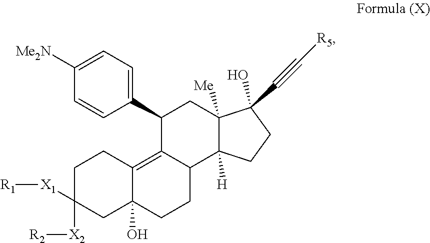

or salts thereof, wherein:
X₁ and X₂ are each independently O or S; R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and R₅ is trialkylsilyl or triakylsilyloxy.

In some embodiments, provided are compounds of Formula (X), or salts thereof, wherein X₁ and X₂ are each independently O or S. In some embodiments, X₁ is O or S. In some embodiments, X₁ is O. In some embodiments, X₁ is S. In some embodiments, X₂ is O or S. In some embodiments, X₂ is O. In some embodiments, X₂ is S. In some embodiments, X₁ and X₂ are both S. In some embodiments, X₁ and X₂ are both O.

In some embodiments, provided are compounds of Formula (X), or salts thereof, wherein R₁ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₁ is a bond. In some embodiments, R₁ is optionally substituted C₁-C₆ alkyl. In some embodiments, R₁ is optionally substituted C₁-C₆ hydroxyalkyl. In some embodiments, R₁ is optionally substituted C₁-C₆ alkoxy. In some embodiments, R₁ is optionally substituted cycloalkyl. In some embodiments, R₁ is optionally substituted cycloheteroalkyl.

In some embodiments, provided are compounds of Formula (X), or salts thereof, wherein R₂ is bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂ is a bond. In some embodiments, R₂ is optionally substituted C₁-C₆ alkyl. In some embodiments, R₂ is optionally substituted C₁-C₆ hydroxyalkyl. In some embodiments, R₂ is optionally substituted C₁-C₆ alkoxy. In some embodiments, R₂ is optionally substituted cycloalkyl. In some embodiments, R₂ is optionally substituted cycloheteroalkyl.

In some embodiments, provided are compounds of Formula (X), or salts thereof, wherein R₁, R₂, X₁, and X₂ are together with the carbon atom connected X₁ and X₂ to form optionally substituted 4-, 5-, 6-, 7-, or 8-membered cyclic ketal or cyclic thioketal. In some embodiments, R₁, R₂, X₁, and X₂ are together with the carbon atom connected X₁ and X₂ to form optionally substituted 4-, 5-, 6-, 7-, or 8-membered cyclic thioketal.

In some embodiments, provided are compounds of Formula (X), or salts thereof, wherein R₁. R₂, X₁, and X₂ are together with the carbon atom connected X₁ and X₂ to form optionally substituted 5-, 6-, 7-, or 8-membered cyclic ketal. In some embodiments, R₁, R₂, X₁, and X₂ are together with the carbon atom connected X₁ and X₂ to form optionally substituted 6-, 7-, or 8-membered cyclic ketal. In some embodiments. R₁, R₂, X₁, and X₂ are together with the carbon atom connected X₁ and X₂ to form optionally substituted 7- or 8-membered cyclic ketal. In some embodiments, R₁, R₂, X₁, and X₂ are together with the carbon atom connected X₁ and X₂ to form optionally substituted 4-, 5-, 6-, or 7-membered cyclic ketal. In some embodiments, R₁, R₂, X₁, and X₂ are together with the carbon atom connected X₁ and X₂ to form optionally substituted 4-, 5-, or 6-membered cyclic ketal. In some embodiments, R₁, R₂, X₁, and X₂ are together with the carbon atom connected X₁ and X₂ to form optionally substituted 4- or 5-membered cyclic ketal. In some embodiments, R₁, R₂, X₁, and X₂ are together with the carbon atom connected X₁ and X₂ to form optionally substituted 5- or 6-membered cyclic ketal. In some embodiments, R₁, R₂, X₁, and X₂ are together with the carbon atom connected X₁ and X₂ to form optionally substituted 4-membered cyclic ketal. In some embodiments. R₁, R₂, X₁, and X₂ are together with the carbon atom connected X₁ and X₂ to form optionally substituted 5-membered cyclic ketal. In some embodiments, R₁, R₂. X₁, and X₂ are together with the carbon atom connected X₁ and X₂ to form optionally substituted 6-membered cyclic ketal. In some embodiments, R₁, R₂, X₁, and X₂ are together with the carbon atom connected X₁ and X₂ to form optionally substituted 7-membered cyclic ketal. In some embodiments, R₁, R₂, X₁, and X₂ are together with the carbon atom connected X₁ and X₂ to form optionally substituted 8-membered cyclic ketal.

In some embodiments, provided are compounds of Formula (X), or salts thereof, wherein R₁ and R₂ are each independently Me, Et, Pr, or Bu. In some embodiments, R₁ is Me, Et, Pr, or Bu. In some embodiments, R₁ is Me. In some embodiments, R₁ is Et. In some embodiments, R₁ is Pr. In some embodiments, R₁ is Bu. In some embodiments, R₂ is Me, Et, Pr, or Bu. In some embodiments, R₂ is Me. In some embodiments, R₂ is Et. In some embodiments, R₂ is Pr. In some embodiments, R₂ is Bu.

In some embodiments, provided are compounds of Formula (X), or salts thereof, wherein M is Li, Na, K, MgBr, CuBr, CuLi, Mg, Cu, or Al. In some embodiments, M is Li. In some embodiments, M is Na. In some embodiments, M is K. In some embodiments, M is MgBr. In some embodiments, M is CuBr. In some embodiments, M is CuLi. In some embodiments, M is Mg. In some embodiments, M is Cu. In some embodiments, M is Al.

In some embodiments, provided are compounds of Formula (X), or salts thereof, wherein n is 1-3. In some embodiments, n is 1-2. In some embodiments, n is 1-2. In some embodiments, n is 2-3. In some embodiments, n is 1. In some embodiments, n is 2. In some embodiments, n is 3.

In some embodiments, provided are compounds of Formula (X), or salts thereof, wherein R₅ is trialkylsilyl or triakylsilyloxy. In some embodiments, R₅ is trialkylsilyl. In some embodiments, R₅ is triakylsilyloxy. In some embodiments, the trialkylsilyl is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS). In some embodiments, the trialkylsilyloxy is trimethylsilyloxy (OTMS), triethylsilyloxy (OTES), tert-butyldimethylsilyloxy (OTBS), tert-butyldiphenylsilyloxy (OTBDPS), thexyldimethylsilyloxy, or triisopropylsilyloxy (OTIPS).

In some embodiments, also provided is a compound of Formula (X) or a salt thereof has a formula of

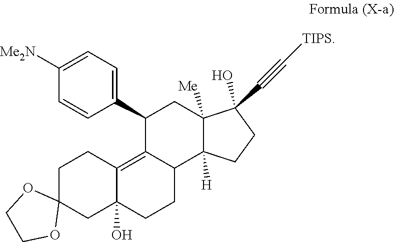

In some embodiments, also provided is a compound of Formula (IV) or a salt thereof has a formula of

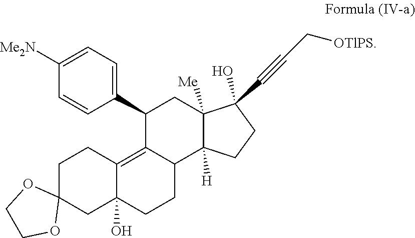

In some embodiments, also provided are compounds having a formula of

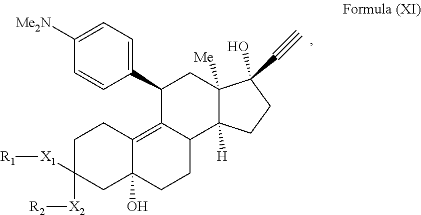

or a pharmaceutically acceptable salt thereof, wherein the variables are as defined herein. In some embodiments, the compound of Formula (XI) has a formula of

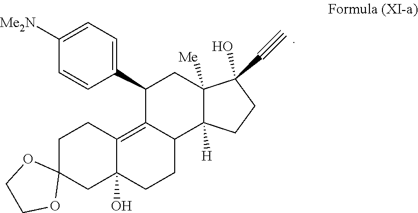 [SMILES: C#C[C@@]1(O)CC[C@H]2C3CC[C@@]4(O)CC5(CCC4=C3[C@@H](c3ccc(N(C)C)cc3)C[C@]21C)OCCO5]

In some embodiments, provided are compounds having a formula of

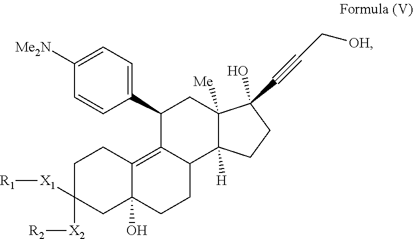

or a pharmaceutically acceptable salt thereof, wherein the variables are as defined herein. In some embodiments, the compound of Formula (V) has a formula of

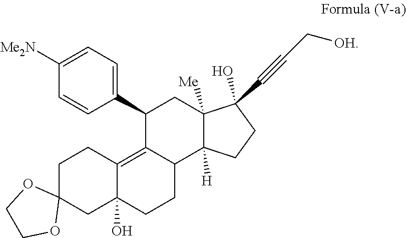 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

In some embodiments, provided are compounds s having a formula of

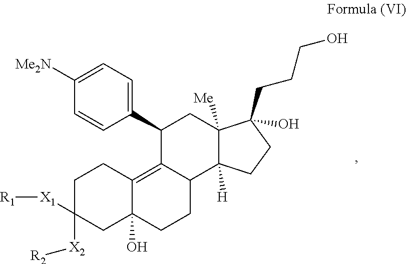

or a pharmaceutically acceptable salt thereof, wherein the variables are as defined herein. In some embodiments, the compound of Formula (VI) has a formula of

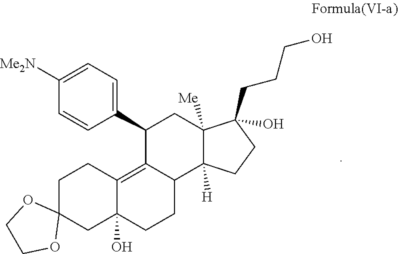 [SMILES: CN(C)c1ccc([C@H]2C[C@]3(C)[C@@H](CC[C@]3(O)CCCO)C3CC[C@@]4(O)CC5(CCC4=C32)OCCO5)cc1]

In some embodiments, provided are processes of preparing compounds of Formulae (II), (III), (IV), (V), (VI), (VII), (IX), (X), (XI), (XII), (II-a), (III-a), (IV-a), (V-a), (VI-a), (X-a), (XI-a), and (XII), or salts there of or pharmaceutically acceptable salts thereof, wherein the contacting is reacting. In some embodiments, the contacting is condensing. In some embodiments, the contacting is oxidizing. In some embodiments, the contacting is reducing. In some embodiments, the contacting is an alkylation. In some embodiments, the contacting is a desilylation. In some embodiments, the contacting is coupling. In some embodiments, the contacting is cyclizing.

In some embodiments, pharmaceutical compositions comprising a compound or pharmaceutically salt thereof of any compound described herein are provided.

The compounds described herein can be made according to the processes described herein and in the examples. The processes described herein can be adapted based upon the compounds desired and described herein. In some embodiments, this method can be used to make one or more compounds as described herein and will be apparent to one of skill in the art which compounds can be made according to the processes described herein.

The conditions and temperatures can be varied, such as shown in the examples described herein. These schemes are non-limiting synthetic schemes and the synthetic routes can be modified as would be apparent to one of skill in the art reading the present specification. The compounds can also be prepared according to the schemes described in the Examples.

In some embodiments, the compounds are made according to schemes described in the examples. The schemes can be used to prepare the compounds and/or intermediates described herein. The conditions and temperatures can be varied or the synthesis can be performed according to the examples described herein with modifications that are readily apparent based upon the compound being synthesized.

The conditions and temperatures can be varied, such as shown in the examples described herein. These schemes are non-limiting synthetic schemes and the synthetic routes can be modified as would be apparent to one of skill in the art reading the present specification.

The present disclosure also provides the following non-limiting embodiments:
1. A process of preparing the compound of Formula (II), or a pharmaceutically acceptable salt thereof, the process comprising:
   contacting the compound of

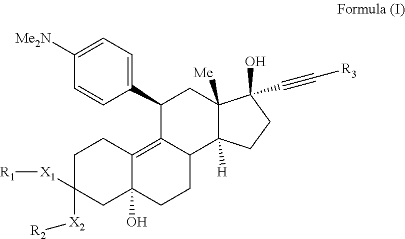

with a base in a first organic solvent under suitable conditions to produce the compound of

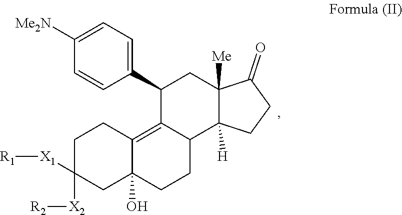

wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₃ is H, optionally substituted C₁-C₆ alkyl, optionally substituted C₁-C₆ hydroxyalkyl, optionally substituted C₁-C₆ alkoxy, optionally substituted cycloalkyl.
2. The process of embodiment 1, wherein both X₁ and X₂ are O.
3. The process according to embodiments 1 or 2, wherein R¹ and R₂ are each independently optionally substituted C₁-C₆ alkyl.
4. The process of any one of embodiments 1-3, wherein R¹ and R₂ are each independently Me, Et, Pr, or Bu.
5. The process of any one of embodiments 1-3, wherein R₁, R₂, X₁, and X₂ are together with the carbon atom connected X₁ and X₂ to form optionally substituted 4-, 5-, 6-, 7-, or 8-membered cyclic ketal.
6. The process of any one of embodiments 1-3, wherein R₁, R₂, X₁, and X₂ are together with the carbon atom connected X₁ and X₂ to form optionally substituted 5- or 6-membered cyclic ketal.
7. The process of any one of embodiments 1-3, wherein R₁, R₂, X₁, and X₂ are together with the carbon atom connected X₁ and X₂ to form optionally substituted 5-membered cyclic ketal.
8. The process of any one of embodiments 1-7, wherein R³ is optionally substituted C₁-C₆ alkyl.
9. The process of any one of embodiments 1-8, wherein R³ is Me.
10. The process of embodiment 1, wherein the compound of Formula (I) has a formula of

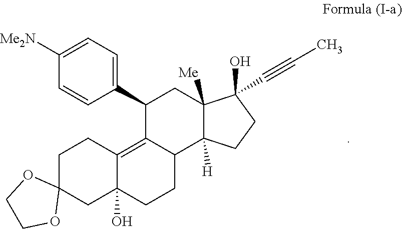 [SMILES: CC#C[C@]1(O)CC[C@H]2C3CC[C@@]4(O)CC5(CCC4=C3[C@@H](c3ccc(N(C)C)cc3)C[C@@]21C)OCCO5]

11. The process of embodiment 10, wherein the compound of Formula (II) has a formula of

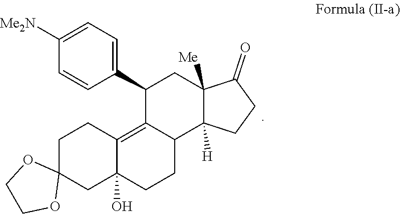 [SMILES: CN(C)c1ccc([C@H]2C[C@]3(C)C(=O)CC[C@H]3C3CC[C@@]4(O)CC5(CCC4=C32)OCCO5)cc1]

12. The process of any one of embodiments 1-11, wherein the base is an alkali metal oxide.
13. The process of any one of embodiments 1-11, wherein the base is tBuOK.
14. The process of any one of embodiments 1-13, wherein the suitable condition comprises heating the mixture of the compound of Formula (I) and the base in the organic solvent to a temperature.
15. The process of embodiment 14, wherein the temperature is at least about 30° C.
16. The process of embodiment 14, wherein the temperature is between about 45° C. and about 70° C.
17. The process of embodiment 14, wherein the temperature is about 55° C.
18. The process of any one of embodiments 14-17, wherein the heated mixture is stirred at the temperature for at least about 5 minutes.
19. The process of embodiment 18, wherein the heated mixture is stirred at the temperature for at least about 30 minutes.
20. The process of any one of embodiments 14-19 further comprising adding aqueous NaCl solution to the reaction mixture.
21. The process of embodiment 20, further comprising cooling the resulting mixture to a temperature between about 0° C. and about 40° C.
22. The process of embodiment 21, wherein the temperature is about 20° C.
23. The process of any one of embodiments 1-21, wherein the first organic solvent is a non-polar organic solvent.
24. The process of embodiment 23, wherein the non-polar organic solvent is toluene, xylene, benzene, cyclohexane, methyl cyclohexane, hexane, heptane, 1,4-dioxane, or a combination thereof.
25. The process of embodiment 23, wherein the non-polar organic solvent is toluene.
26. The process of embodiment 20 further comprising separating the resulting organic phase and washing with aqueous NaCl solution.
27. The process of embodiment 26 further comprising evaporating the organic phase to form a residue.
28. The process of embodiment 27 wherein the residue is purified by precipitation.
29. The process of embodiment 28, wherein the precipitation comprises:
   dissolving the residue in a first solvent to form a mixture;
   heating the mixture to a temperature;
   adding a second solvent to the heated mixture to form a precipitate; and
   collecting the precipitate by filtration to produce the compound of Formula (II).
30. The process of embodiment 29, wherein the first solvent is an alcohol.
31. The process of embodiment 30, wherein the alcohol is methanol, ethanol, propanol, isopropanol, butanol, or a combination thereof
32 The process of embodiment 31, wherein the alcohol is methanol.
33. The process of any of embodiments 29-31, wherein the mixture is heated to a temperature of about 55° C.
34 The process of any of embodiments 29-33, wherein the heated mixture is stirred for at least about 5 minutes.
35. The process of any of embodiments 29-33, wherein the heated mixture is stirred for at least about 20 minutes.
36. The process of any of embodiments 29-35, wherein the second solvent is water, an ether, an alkane, an ester, a ketone, or a combination thereof.
37 The process of embodiment 36, wherein the alkane is butane, pentane, hexane, heptane, octane, or a combination thereof.
38. The process of embodiment 36, wherein the ketone is acetone.
39. The process of embodiment 36, wherein the ether is diethyl ether, diisopropyl ether, cyclopentyl methyl ether, methyl tert butyl ether, or a combination thereof.
40 The process of any of embodiments 29-35, wherein the second solvent is water.
41 The process of embodiment 40, wherein the volume ratio of water to methanol is about 3 to 1.
42. The process of any of embodiments 29-41, wherein the filtered precipitate is dried under vacuum between about 40° C. and about 90° C.
43. A process of preparing a compound of Formula (III), or a pharmaceutically acceptable salt thereof, the process comprising:
   irradiating a solution of the compound of

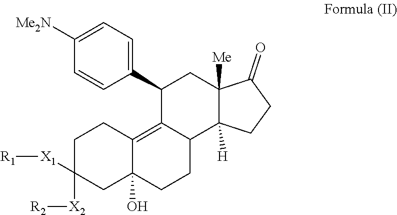

in an organic solvent with a UV light in a flow reactor to produce the compound of

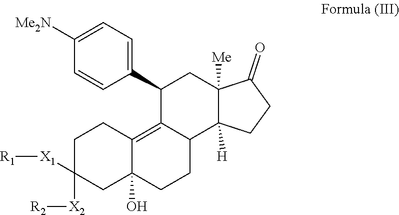

wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal.
44 The process of embodiment 43, wherein both X₁ and X₂ are O.
45. The process according to embodiments 43 or 44, wherein R¹ and R₂ are each independently optionally substituted C₁-C₆ alkyl.
46. The process of any one of embodiments 43-45, wherein R₁ and R₂ are each independently Me, Et, Pr, or Bu.
47. The process of any one of embodiments 43 or 44, wherein R₁, R₂, X₁, and X₂ are together with the carbon atom connected X₁ and X₂ to form optionally substituted 4-, 5-, 6-, 7-, or 8-membered cyclic ketal.
48. The process according to embodiments 43 or 44, wherein R₁, R₂, X₁, and X₂ are together with the carbon atom connected X₁ and X₂ to form optionally substituted 5- or 6-membered cyclic ketal.
49. The process according to embodiments 43 or 44, wherein R₁, R₂, X₁, and X₂ are together with the carbon atom connected X₁ and X₂ to form optionally substituted 5-membered cyclic ketal.
50. The process of embodiment 43, wherein the compound of Formula (II) has a formula of

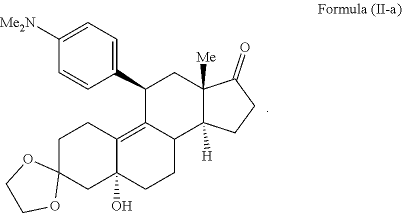 [SMILES: CN(C)c1ccc([C@H]2C[C@]3(C)C(=O)CC[C@H]3C3CC[C@@]4(O)CC5(CCC4=C32)OCCO5)cc1]

51. The process of embodiment 50, wherein the compound of Formula (III) has a formula of

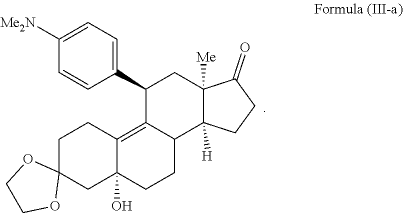 [SMILES: CN(C)c1ccc([C@H]2C[C@@]3(C)C(=O)CC[C@H]3C3CC[C@@]4(O)CC5(CCC4=C32)OCCO5)cc1]

52. The process of any one of embodiments 43-51, wherein the UV light is a narrow band frequency light at a wavelength from about 300 nm to about 320 nm.
53. The process of any one of embodiments 43-52, wherein the UV light is a narrow band frequency light with a wavelength at about 311 nm.
54. The process of any one of embodiments 43-53, wherein the UV light is from a low-pressure mercury lamp.
55. The process of any one of embodiments 43-54, wherein the low-pressure mercury lamp is fitted inside the flow reactor.
56. The process of any one of embodiments 43-55, wherein the flow reactor is fabricated from a long polytetrafluoroethylene (PTFE) tubing with an inner diameter between 1 to 20 millimeters.
57. The process of any one of embodiments 43-55, wherein the flow reactor is fabricated from a long fluorinated ethylene propylene (FEP) tubing with an inner diameter between 1 to 20 millimeters.
58. The process of any one of embodiments 43-55, wherein the flow reactor is fabricated from a long perfluoroalkoxy alkane (PFA) tubing with an inner diameter between 1 to 20 millimeters.
59. The process of any one of embodiments 43-58, wherein the organic solvent is ethyl acetate, toluene, methyltetrahydrofuran, tetrahydrofuran, isopropyl ether, methanol, water, dichloromethane, isopropylether, or a combination thereof.
60. The process of any one of embodiments 43-58, wherein the organic solvent is ethyl acetate.
61. The process of any one of embodiments 43-60 further comprising washing the solution with aqueous NaHSO₃ solution to remove the aldehyde impurities from the UV irradiation.
62. The process of any one of embodiments 43-61, further comprising purifying the crude compound of Formula (III) with solid absorbents such as silica gel, magnesium silicate, alumina, polymers, clays, or other porous or high surface area solids.
63. The process of any one of embodiments 43-62 further comprising recrystallizing the compound of Formula (III).
64 The process of embodiment 63, wherein the compound of Formula (III) is recrystallized from ethyl acetate and heptane.
65. The process of embodiment 64, wherein the volume ratio of ethyl acetate to heptane is about 1 to 3.
66. A process of preparing a compound of Formula (X), or a salt thereof, the process comprising:
   contacting the compound of

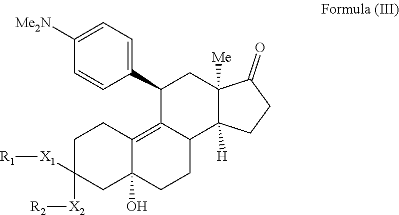

with an organometallic reagent having a formula of

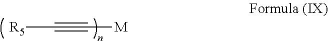

to produce the compound of

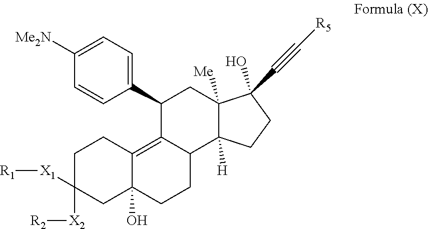

or a salt form thereof; and
wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
   R₅ is trialkylsilyl or triakylsilyloxy.
67. The process of embodiment 66, wherein both X₁ and X₂ are O.
68. The process according to embodiments 66 or 67, wherein R¹ and R₂ are each independently optionally substituted C₁-C₆ alkyl.
69 The process of any one of embodiments 66-68, wherein R¹ and R₂ are each independently Me, Et, Pr, or Bu.
70. The process according to embodiments 66 or 67, wherein R₁, R₂, X₁, and X₂ are together with the carbon atom connected X₁ and X₂ to form optionally substituted 4-, 5-, 6-, 7-, or 8-membered cyclic ketal.
71 The process according to embodiments 66 or 67, wherein R₁, R₂, X₁, and X₂ are together with the carbon atom connected X₁ and X₂ to form optionally substituted 5- or 6-membered cyclic ketal.
72. The process according to embodiments 66 or 67, wherein R₁, R₂, X₁, and X₂ are together with the carbon atom connected X₁ and X₂ to form optionally substituted 5-membered cyclic ketal.
73. The process of any one of embodiments 66-72, wherein the organometallic reagent of Formula IX is nucleophilic.
74 The process of embodiment 73, wherein the organometallic reagent of Formula IX is an organolithium reagent.
75 The process of embodiment 74, wherein the organolithium reagent

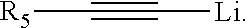

76. The process of embodiment 75 further comprising preparing the organolithium reagent by contacting

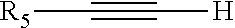

with an alkyllithium reagent in a first organic solvent at a low temperature.
77. The process of embodiment 76, wherein the alkyllithium reagent is n-butyllithum, n-heptanyllithoum or n-hexyllithium, or a combination thereof.
78 The process of embodiment 76, wherein the alkyllithium reagent is n-hexyllithium.
79. The process of embodiment 76, wherein the low temperature is below −10° C.
80 The process of embodiment 76, wherein the low temperature is about −25° C.
81 The process of any one of embodiments 73-80, wherein the compound of Formula (III) or a salt form thereof is separately dissolved in a second organic solvent before contacting the organolithium reagent at the low temperature.
82. The process of embodiment 81, wherein the compound of Formula (III) or a salt form thereof in the second organic solvent is added dropwise to the organolithium reagent in the first organic solvent at the low temperature.
83. The process of embodiment 82, wherein both the first and second organic solvents are polar non-protic organic solvents.
84. The process of embodiment 83, wherein both the first and second organic solvents are tetrahydrofuran or 2-methyltetrohydrafuran.
85 The process of embodiment 83, wherein both the first and second organic solvents are 2-methyltetrohydrafuran.
86 The process of any one of embodiments 73-85, wherein the molar ratio of the organolithium reagent to the compound of Formula (III) is between about 1:1 to about 10:1.
87. The process of any one of embodiments 73-85, wherein the molar ratio of the organolithium reagent to the compound of Formula (III) is about 4:1.
88. The process of any one of embodiments 73-87, wherein a diastereofacial selectivity for the compound of Formula (X) to the 17-β-hydroxy-epimer thereof is achieved.
89. The process of embodiment 88, wherein the diastereofacial selectivity of the compound of Formula (X) to the 17-β-hydroxy-epimer thereof, is more than about 1:1.
90. The process of embodiment 88, wherein the diastereofacial selectivity of the compound of Formula (X) to the 17-β-hydroxy-epimer thereof, is about 8:1.
91. The process of embodiment 88, wherein the diastereofacial selectivity of the compound of Formula (X) to the 17-β-hydroxy-epimer thereof, is about 9.3:1.
92 The process of embodiment 88, wherein the diastereofacial selectivity of the compound of Formula (X) to the 17-β-hydroxy-epimer thereof, is about 13.4:1.
93. The process of embodiment 88, wherein the diastereofacial selectivity of the compound of Formula (X) to the 17-β-hydroxy-epimer thereof, is about 18.8:1.
94. The process of any one of embodiments 73-92, further comprising quenching the resulting mixture with ice water to form an aqueous phase and an organic phase; optionally washing the organic phase with water and brine; and optionally drying the organic phase with a drying reagent to produce a solution of the compound of Formula (X) in 2-methyltetrahydrofuran.
95. The process of embodiment 94, wherein the drying reagent is anhydrous Na₂SO₄, MgSO₄, or CaSO₄.
96 The process of embodiment 94, wherein the drying reagent is anhydrous Na₂SO₄.
97. The process of any one of embodiments 66-96, the process further comprising: contacting the solution of the compound of Formula (X) in 2-methyltetrahydrofuran with a fluoride-containing reagent to produce the compound having a formula of

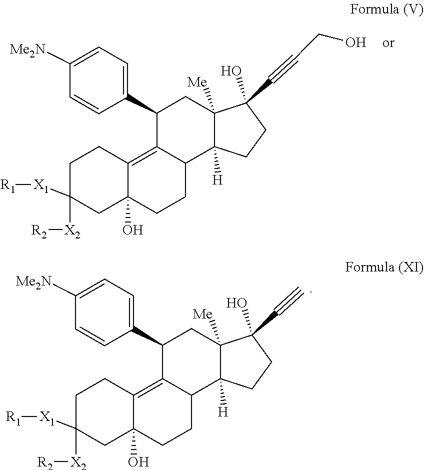

98. The process of embodiment 97, wherein the fluoride-containing reagent is tetra-n-butylammonium fluoride (TBAF), NH₄F, KH₂F, KF, NaF, hydrogen fluoride, hydrogen fluoride pyridine, tris(dimethylamino)sulfonium difluorotrimethylsilicate, triethylamine trihydrofluoride (Et₃N·3HF), tetrabutylammonium tetra(t-butyl alcohol)-coordinated fluoride (TBAF(tBuOH)₄), tetrabutylammonium triphenyldifluorosilicate, or a combination thereof.
99 The process of embodiment 97, wherein the fluoride-containing reagent is tetra-n-butylammonium fluoride (TBAF), NH₄F, KH₂F, KF, or a combination thereof.
100. The process of embodiment 97, wherein the fluoride-containing reagent is tetra-n-butylammonium fluoride (TBAF).
101. The process of embodiment 97, wherein the fluoride-containing reagent is NH₄F.
102. The process of embodiment 97, wherein the fluoride-containing reagent is KH₂F.
103. The process of embodiment 97, wherein the fluoride-containing reagent is KF.
104. The process of embodiment 97, wherein the fluoride-containing reagent is NaF.
105. The process of any one of embodiments 97-104, wherein the fluoride-containing reagent is added to the solution of the compound of Formula (X) in 2-methyltetrahydrofuran to form a mixture.
106. The process of embodiment 105, wherein the mixture is optionally cooled to a temperature and kept at the temperature for a period of time.
107. The process of embodiment 106, wherein the temperature is at least about 10° C.
108. The process of embodiment 106, wherein the temperature is about 15° C.
109. The process of embodiment 106, wherein the temperature is about 25° C.
110. The process of any one of embodiments 106-109, wherein the period of time is at least about 5 minutes.
111. The process of any one of embodiments 106-109, wherein the period of time is about 16 hours.
112. The process of any one of embodiments 106-111, wherein the mixture is optionally washed with water and brine, optionally dried over a drying reagent, and evaporated to form the compound having a formula of Formula (V) or Formula (XI) with the 17-β-hydroxy-epimer thereof as an impurity.
113. The process of embodiment 112, wherein the dried mixture is evaporated below 45° C. under vacuum.
114. The process of embodiment 112, wherein the molar ratio of the compound of Formula (V) and the 17-β-hydroxy-epimer thereof is about 10:1.
115. The process of embodiment 112, wherein the molar ratio of the compound of Formula (XI) and the 17-β-hydroxy-epimer thereof is about 15.2:1.
116. The process of any one of embodiments 66-115, wherein R₅ is trialkylsilyl.
117. The process of any one of embodiments 66-116, wherein R₅ is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS).
118. The process of any one of embodiments 66-117, wherein R₅ is triisopropylsilyl (TIPS).
119. The process of any one of embodiments 97-118, wherein the process further comprising:
   contacting the compound of

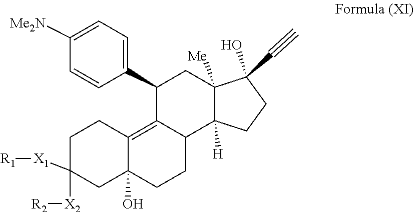

with paraformaldehyde, at least one bases, and CuI in a fourth organic solvent under suitable conditions to produce the compound of

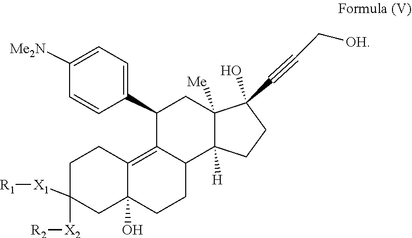

120. The process of embodiment 119, the process further comprising two bases.
121. The process of embodiment 120, wherein the two bases are a trialkylamine and an alkali hydroxide.
122. The process of embodiment 121, wherein the trialkylamine is triethylamine.
123. The process according to embodiments 120 or 121, wherein the alkali hydroxide is KOH.
124. The process of embodiment 119, wherein the fourth organic solvent is a non-protic organic solvent.
125. The process of embodiment 124, wherein the non-protic organic solvent is a polar non-protic organic solvent.
126. The process of embodiment 125, wherein the polar non-protic organic solvent is dimethyl sulfoxide.
127. The process of any one of embodiments 119-126, wherein the diastereofacial selectivity of the compound of Formula (V) to the 17-β-hydroxy-epimer thereof is at least about 9:1.
128. The process of any one of embodiments 119-126, wherein the diastereofacial selectivity of the compound of Formula (V) to the 17-β-hydroxy-epimer thereof is about 15.3:1.
129. The process of any one of embodiments 66-128, wherein the compound of Formula (III) has a formula of

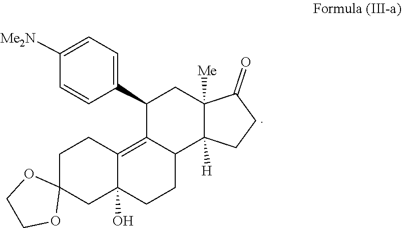 [SMILES: CN(C)c1ccc([C@H]2C[C@@]3(C)C(=O)CC[C@H]3C3CC[C@@]4(O)CC5(CCC4=C32)OCCO5)cc1]

130. The process of embodiment 129, wherein the compound of Formula (X) has a formula of

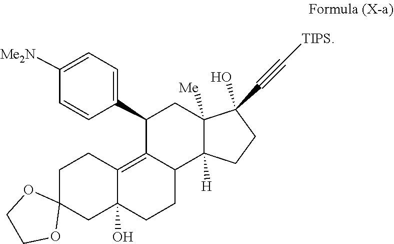

131. The process of any one of embodiments 97-130, wherein the compound of Formula (XI) has a formula of

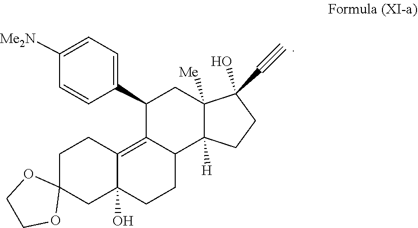 [SMILES: C#C[C@@]1(O)CC[C@H]2C3CC[C@@]4(O)CC5(CCC4=C3[C@@H](c3ccc(N(C)C)cc3)C[C@]21C)OCCO5]

132. The process of any one of embodiments 66-115, wherein the compound of Formula (X) or a salt form thereof, has a formula of

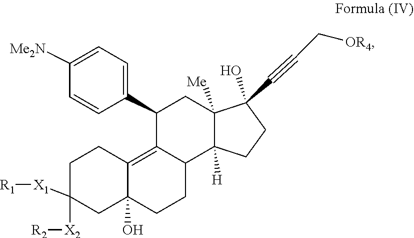

wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₄ is trialkylsilyl.
133. The process of embodiment 132, wherein R⁴ is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS).
134. The process of embodiment 132, wherein R⁴ is triisopropylsilyl (TIPS).
135. The process of any one of embodiments 132-134, wherein the compound of Formula (III) has a formula of

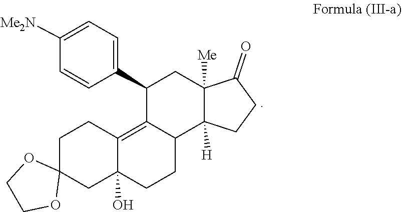 [SMILES: CN(C)c1ccc([C@H]2C[C@@]3(C)C(=O)CC[C@H]3C3CC[C@@]4(O)CC5(CCC4=C32)OCCO5)cc1]

136. The process of embodiment 135, wherein the compound of Formula (IV) has a formula of

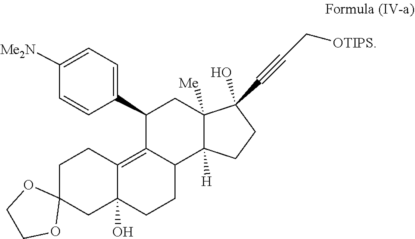

137. The process of any one of embodiments 97-115 and 132-136, wherein the compound of Formula (V) has a formula of

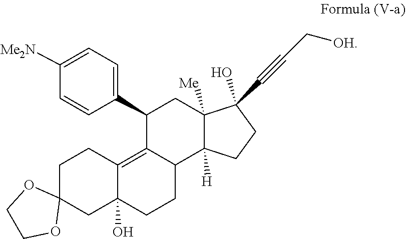 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

138. The process of any one of embodiments 97-137, further comprising purifying the compound of Formula (V) from the 17-β-hydroxy-epimer thereof comprising forming an aggregate of the compound of Formula (V) with a salt.
139. The process of embodiment 138, wherein the salt is a citrate salt or an acetate salt.
140. The process of embodiment 138, wherein the salt is a citrate salt.
141. The process of embodiment 140, wherein the citrate salt is lithium citrate.
142. The process of embodiment 138, wherein the salt is an acetate salt.
143. The process of embodiment 142, wherein the acetate salt is potassium acetate.
144. The process of any one of embodiments 138-143, wherein the molar ratio of the compound of Formula (V) to the 17-β-hydroxy-epimer thereof in the aggregate is more than about 20:1, about 40:1, about 60:1, about 80:1, about 100:1, about 150:1, about 200:1, about 250:1, about 300:1, about 350:1, or about 400:1.
145. The process of any one of embodiments 138-144, wherein the aggregate is formed in a mixture of a polar non-protic organic solvent and a non-polar organic solvent.
146. The process of embodiment 145, wherein the volume ratio of the polar non-protic organic solvent to the non-polar organic solvent in the mixture is between about 1:1 to about 4.4:1.
147. The process of embodiment 145, wherein the volume ratio of the polar non-protic organic solvent to the non-polar organic solvent in the mixture is about 1.4:1.
148. The process of any one of embodiments 145 or 146, wherein the polar non-protic organic solvent is ethyl acetate.
149. The process of any one of embodiments 145-148, wherein the non-polar organic solvent is heptane.
150. The process of any one of embodiments 138-149, further comprising dissolving the aggregate in the polar non-protic organic solvent to form an organic phase, washing the organic phase with water, and concentrating the organic phase to form the compound of Formula (V) in high purity.
151. The process of embodiment 150, wherein the purity of the compound of Formula (V) is at least about 90%, at least about 92%, at least about 94%, at least about 96%, at least about 98%, or at least about 99%.
152. A process of preparing a compound of Formula (VII), or a pharmaceutically acceptable salt thereof, the process comprising:
   a) subjecting the compound of

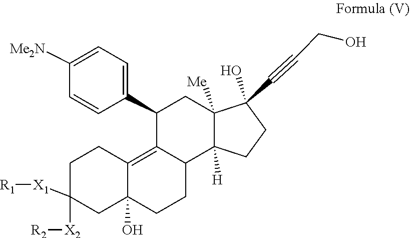

or a salt form thereof, prepared according to any process of embodiments 97-151 under a hydrogenation condition to produce the compound of

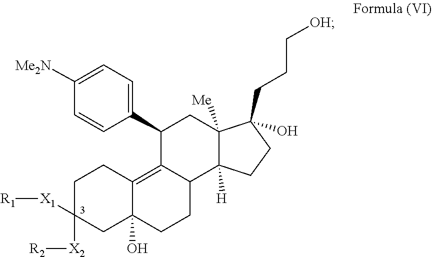

and
e) hydrolyzing the compound of Formula (VI) under a hydrolysis condition to produce the compound of

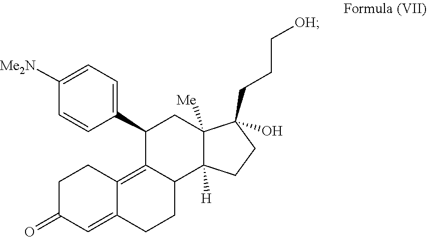 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt
153. The process of embodiment 152, wherein the hydrogenation condition is suitable for converting an unsaturated carbon-carbon triple bond to a saturated carbon-carbon bond.
154. The process of embodiment 153, wherein the suitable hydrogenation condition comprises hydrogen and a metal catalyst.
155. The process of embodiment 153, wherein the suitable hydrogenation condition comprises hydrogen, a metal catalyst, and an alkali metal borohydride.
156. The process of embodiment 155, wherein the alkali metal borohydride is sodium borohydride or potassium borohydride.
157. The process of any of embodiments 154-156, wherein the metal catalyst comprises palladium.
158. The process of embodiment 152, wherein the hydrolysis condition is a suitable condition for removing the protection for the 3-ketone of Formula (VI).
159. The process of embodiment 158, wherein the suitable hydrolysis condition comprises an alkali metal bisulfate.
160. The process of embodiment 159, wherein the alkali metal bisulfate is potassium bisulfate.
161. The process of any one of embodiments 152-161, further comprising recrystallizing the compound of Formula (VII).
162. The process of embodiment 161, wherein the compound of Formula (VII) is recrystallized from a mixture of at least one polar solvent and/or at least one non-polar solvent.
163. The process of embodiment 161, wherein the compound of Formula (VII) is recrystallized from a mixture of at least one polar solvent and at least one non-polar solvent.
164. The process of embodiment 161, wherein the compound of Formula (VII) is recrystallized from a mixture of one polar solvent and one non-polar solvent.
165. The process of any one of embodiments 162-164, wherein the polar solvent is ethyl acetate.
166. The process of any one of embodiments 162-165, wherein the polar solvent is n-heptane.
167. The process of embodiment 162, wherein the mixture is a mixture of ethyl acetate and n-heptane.
168. The process of embodiment 167, wherein the volume ratio of ethyl acetate to n-heptane is about 1 to 3.
169. A process of preparing a compound of Formula (XII), or a pharmaceutically acceptable salt thereof, the process comprising:
   subjecting the compound of

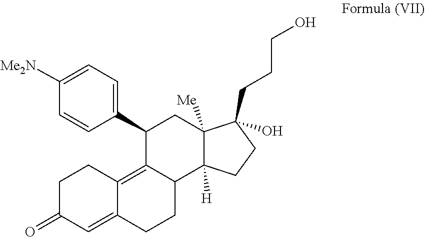 [SMILES: CN(C)c1ccc([C@H]2C[C@]3(C)[C@@H](CC[C@]3(O)CCCO)C3CCC4=CC(=O)CCC4=C32)cc1]

or pharmaceutically acceptable salt thereof, prepared according to any process of embodiments 152-155 under a suitable N-demethylation condition to produce the
compound of

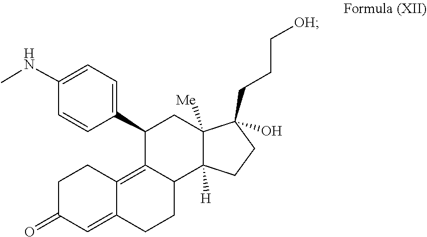 [SMILES: CNc1ccc([C@H]2C[C@]3(C)[C@@H](CC[C@]3(O)CCCO)C3CCC4=CC(=O)CCC4=C32)cc1]

or a pharmaceutically acceptable salt.
170. The process of embodiment 169, wherein the suitable N-demethylation condition is suitable for removing a methyl group from the dimethylamino phenyl group on the compound of Formula (VII).
171. The process according embodiments 169 or 170, wherein the suitable N-demethylation condition is a von Braun reaction, a method of applying chloroformate, a Polonovski reaction, a photochemical method, a microbial method, or an enzymatic method.
172. The process of embodiment 171, wherein the enzymatic method is a cytochrome P450 enzyme mediated N-demethylation.
173. The process of embodiment 171, wherein the enzymatic method is a cytochrome P450 3A-mediated N-demethylation.
174. A compound having a formula of

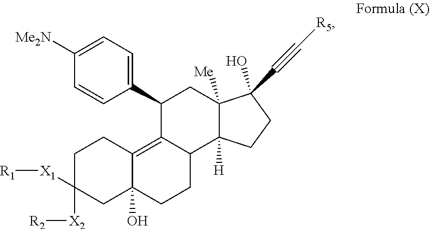

or a pharmaceutically acceptable salt thereof,
wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₅ is trialkylsilyl or triakylsilyloxy.
175. The compound of embodiment 174, or a pharmaceutically acceptable salt thereof, wherein both X₁ and X₂ are O.
176. The compound according to embodiments 174 or 175, or a pharmaceutically acceptable salt thereof, wherein R¹ and R₂ are each independently optionally substituted C₁-C₆ alkyl.
177. The compound of any one of embodiments 174-176, or a pharmaceutically acceptable salt thereof, wherein R¹ and R₂ are each independently Me, Et, Pr, or Bu.
178. The compound of any one of embodiments 174-176, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
179. The compound of any one of embodiments 174-176, or a pharmaceutically acceptable salt thereof, wherein R₁, R₂, X₁, and X₂ are together with the carbon atom connected X₁ and X₂ to form optionally substituted 5- or 6-membered cyclic ketal.
180. The compound of any one of embodiments 174-176, or a pharmaceutically acceptable salt thereof, wherein R₁, R₂, X₁, and X₂ are together with the carbon atom connected X₁ and X₂ to form optionally substituted 5-membered cyclic ketal.
181. The compound of any one of embodiments 174-180, wherein R₅ is trialkylsilyl.
182. The compound of any one of embodiments 174-180, wherein R₅ is trimethylsilyl (TMS), triethylsilyl (TES), tert-butyldimethylsilyl (TBS), tert-butyldiphenylsilyl (TBDPS), or triisopropylsilyl (TIPS).
183. The compound of any one of embodiments 174-180, wherein R₅ is triisopropylsilyl (TIPS).
184. The compound of embodiment 174, or a pharmaceutically acceptable salt thereof, wherein the compound of Formula (X) has a formula of

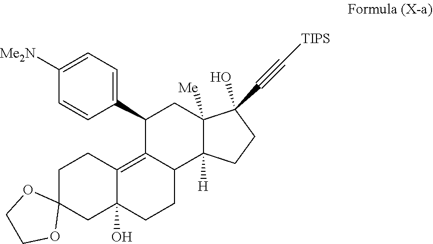

185. The compound of any one of embodiments 174-180, wherein R₅ is triakylsilyloxy.
186. The compound of any one of embodiments 174-180, wherein R₅ is trimethylsilyloxy (OTMS), triethylsilyloxy (OTES), tert-butyldimethylsilyloxy (OTBS), tert-butyldiphenylsilyloxy (OTBDPS), or triisopropylsilyloxy (OTIPS).
187. The compound of any one of embodiments 174-180, wherein R₅ is triisopropylsilyloxy (OTIPS).
188. The compound of embodiment 174, or a pharmaceutically acceptable salt thereof, wherein the compound of Formula (X) has a formula of

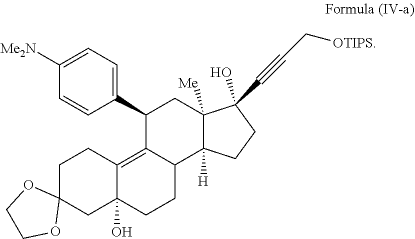

189. A compound having a formula of

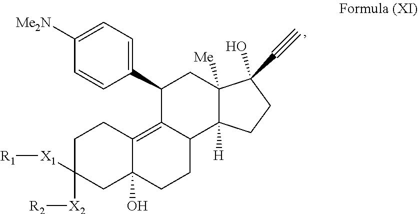

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
190. The compound of embodiment 189, or a pharmaceutically acceptable salt thereof, wherein both X₁ and X₂ are O.
191. The compound according to embodiments 189 or 190, or a pharmaceutically acceptable salt thereof, wherein R¹ and R₂ are each independently optionally substituted C₁-C₆ alkyl.
192. The compound of any one of embodiments 189-191, or a pharmaceutically acceptable salt thereof, wherein R¹ and R₂ are each independently Me, Et, Pr, or Bu.
193. The compound according to embodiments 189 or 190, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
194. The compound according to embodiments 189 or 190, or a pharmaceutically acceptable salt thereof, wherein R₁, R₂, X₁, and X₂ are together with the carbon atom connected X₁ and X₂ to form optionally substituted 5-membered cyclic ketal.
195. The compound of embodiment 189, or a pharmaceutically acceptable salt thereof, wherein the compound of Formula (XI) has a formula of

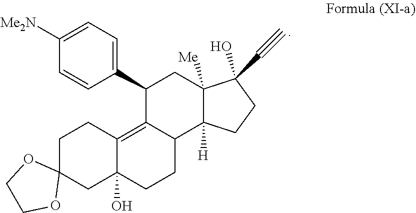 [SMILES: C#C[C@@]1(O)CC[C@H]2C3CC[C@@]4(O)CC5(CCC4=C3[C@@H](c3ccc(N(C)C)cc3)C[C@]21C)OCCO5]

196. A compound having a formula of

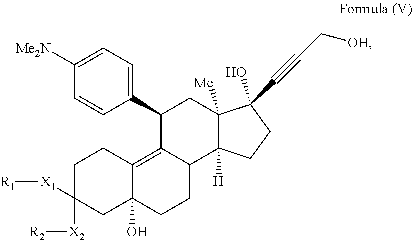

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
197. The compound of embodiment 196, or a pharmaceutically acceptable salt thereof, wherein both X₁ and X₂ are O.
198. The compound according to embodiments 196 or 197, or a pharmaceutically acceptable salt thereof, wherein R¹ and R₂ are each independently optionally substituted C₁-C₆ alkyl.
199. The compound of any one of embodiments 196-198, or a pharmaceutically acceptable salt thereof, wherein R¹ and R₂ are each independently Me, Et, Pr, or Bu.
200. The compound according to embodiments 196 or 197, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
201. The compound according to embodiments 196 or 197, or a pharmaceutically acceptable salt thereof, wherein R₁, R₂, X₁, and X₂ are together with the carbon atom connected X₁ and X₂ to form optionally substituted 5-membered cyclic ketal.
202. The compound of embodiment 196, or a pharmaceutically acceptable salt thereof, wherein the compound of Formula (V) has a formula of

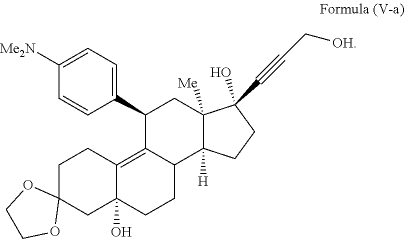 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

203. A compound having a formula of

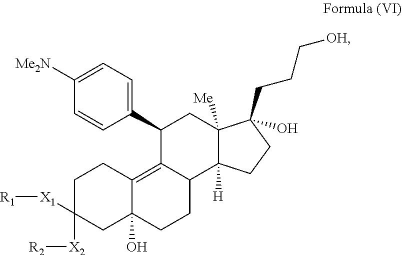

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
204. The compound of embodiment 203, or a pharmaceutically acceptable salt thereof, wherein both X₁ and X₂ are O.
205. The compound according to embodiments 203 or 204, or a pharmaceutically acceptable salt thereof, wherein R¹ and R₂ are each independently optionally substituted C₁-C₆ alkyl.
206. The compound of any one of embodiments 203-205, or a pharmaceutically acceptable salt thereof, wherein R¹ and R₂ are each independently Me, Et, Pr, or Bu.
207. The compound according to embodiments 203 or 204, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
208. The compound according to embodiments 203 or 204, or a pharmaceutically acceptable salt thereof, wherein R₁, R₂, X₁, and X₂ are together with the carbon atom connected X₁ and X₂ to form optionally substituted 5-membered cyclic ketal.
209. The compound of embodiment 203, or a pharmaceutically acceptable salt thereof,

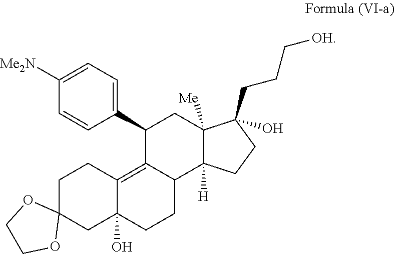 [SMILES: CN(C)c1ccc([C@H]2C[C@]3(C)[C@@H](CC[C@]3(O)CCCO)C3CC[C@@]4(O)CC5(CCC4=C32)OCCO5)cc1]

wherein the compound of Formula (VI) has a formula of
210. A process of preparing the compound of Formula (II), or a pharmaceutically acceptable salt thereof, the process comprising:
   contacting the compound of

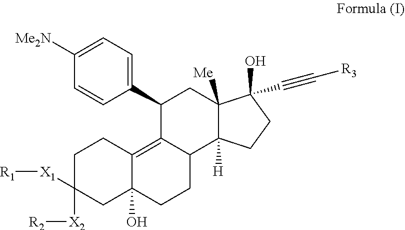

with a base in a first organic solvent under suitable conditions to produce the compound of

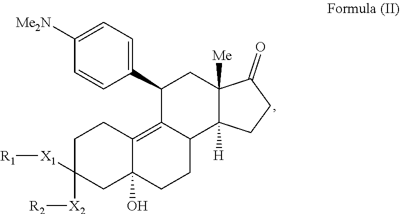

wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₃ is H, optionally substituted C₁-C₆ alkyl, optionally substituted C₁-C₆ hydroxyalkyl, optionally substituted C₁-C₆ alkoxy, optionally substituted cycloalkyl.
211. The process of embodiment 210, wherein both X₁ and X₂ are O.
212. The process according to embodiments 210 or 211, wherein R¹ and R₂ are each independently optionally substituted C₁-C₆ alkyl.
213. The process of any one of embodiments 210-212, wherein R¹ and R₂ are each independently Me, Et, Pr, or Bu.
214. The process of any one of embodiments 210-212, wherein R₁, R₂, X₁, and X₂ are together with the carbon atom connected X₁ and X₂ to form optionally substituted 4-, 5-, 6-, 7-, or 8-membered cyclic ketal.
215. The process of any one of embodiments 210-212, wherein R₁, R₂, X₁, and X₂ are together with the carbon atom connected X₁ and X₂ to form optionally substituted 5- or 6-membered cyclic ketal.
216. The process of any one of embodiments 210-212, wherein R₁, R₂, X₁, and X₂ are together with the carbon atom connected X₁ and X₂ to form optionally substituted 5-membered cyclic ketal.
217. The process of any one of embodiments 210-216, wherein R³ is optionally substituted C₁-C₆ alkyl.
218. The process of any one of embodiments 210-217, wherein R³ is Me.
219. The process of embodiment 210, wherein the compound of Formula (I) has a formula of

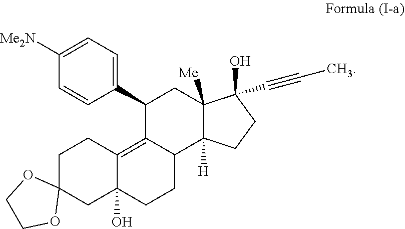 [SMILES: CC#C[C@]1(O)CC[C@H]2C3CC[C@@]4(O)CC5(CCC4=C3[C@@H](c3ccc(N(C)C)cc3)C[C@@]21C)OCCO5]

220. The process of embodiment 219, wherein the compound of Formula (II) has a formula of

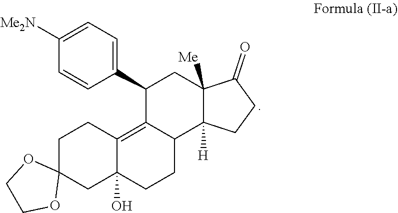 [SMILES: CN(C)c1ccc([C@H]2C[C@]3(C)C(=O)CC[C@H]3C3CC[C@@]4(O)CC5(CCC4=C32)OCCO5)cc1]

221. The process of any one of embodiments 210-220, wherein the base is an alkali metal oxide.
222. The process of any one of embodiments 210-220, wherein the base is tBuOK.
223. The process of any one of embodiments 210-222, wherein the suitable condition comprises heating the mixture of the compound of Formula (I) and the base in the organic solvent to a temperature.
224. The process of embodiment 223, wherein the temperature is at least about 30° C.
225. The process of embodiment 223, wherein the temperature is between about 60° C. and about 80° C.
226. The process of embodiment 223, wherein the temperature is about 70° C.
227. The process of any one of embodiments 223-226, wherein the heated mixture is stirred at the temperature for at least about 5 minutes.
228. The process of embodiment 227, wherein the heated mixture is stirred at the temperature for at least about 30 minutes.
229. The process of any one of embodiments 223-228 further comprising cooling the heated mixture to room temperature.
230. The process of any one of embodiments 210-229, wherein the first organic solvent is a polar organic solvent.
231. The process of embodiment 230, wherein the polar organic solvent is dimethylformamide, diethylformamide, 1-butanol, 2-butanol, iso-butanol, tert-butanol, or a combination thereof.
232. The process of embodiment 230, wherein the polar organic solvent is dimethylformamide.
233. The process of embodiment 23 further comprising adding NH₄Cl aqueous solution to the resulting mixture to form a suspension.
234. The process of embodiment 229 further comprising filtering the suspension to yield a solid.
235. The process of embodiment 234 further comprising crystallizing the solid to produce the compound of Formula (II).
236. The process of embodiment 235, wherein the solid is crystallized in a solvent.
237. The process of embodiment 236, wherein the solvent is an alcohol, H₂O, an ether, an alkane, an ester, a ketone, or a combination thereof.
238. The process of embodiment 237, wherein the alkane is butane, pentane, hexane, heptane, octane, or a combination thereof.
239. The process of embodiment 237, wherein the alkane is heptane.
240. The process of embodiment 237, wherein the ketone is acetone.
241. The process of embodiment 237, wherein the ester is ethyl acetate.
242. The process of embodiment 237, wherein the solvent is a combination of an alcohol and an ether.
243. The process of embodiment 242, wherein the alcohol is methanol, ethanol, propanol, isopropanol, butanol, or a combination thereof.
244. The process of embodiment 243, wherein the alcohol is isopropanol.
245. The process of embodiment 242, wherein the ether is diethyl ether, diisopropyl ether, cyclopentyl methyl ether, methyl tert-butyl ether, or a combination thereof.
246. The process of embodiment 245, wherein the ether is diisopropyl ether.
247. The process of any one of embodiments 243-246, wherein the volume ratio of the alcohol to the ether is about 1:1.
248. The process of embodiment 230, wherein the polar organic solvent is tert-butanol.
249. The process of embodiment 233 further comprising adding water to the cooled mixture and extracting the resulting aqueous phase with a second organic solvent to produce the compound of Formula (II).
250. The process of embodiment 249, wherein the second organic solvent is a polar organic solvent.
251. The process of embodiment 249, wherein the polar organic solvent is a non-protic polar organic solvent.
252. The process of embodiment 251, wherein the non-protic polar organic solvent is ethyl acetate.
253. A process of preparing a compound of Formula (III), or a pharmaceutically acceptable salt thereof, the process comprising:
   irradiating a solution of the compound of

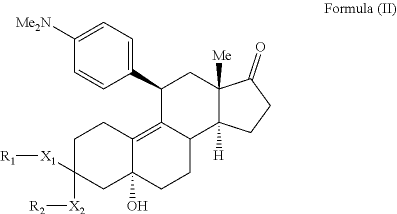

in an organic solvent with a UV light in a flow reactor to produce the compound of

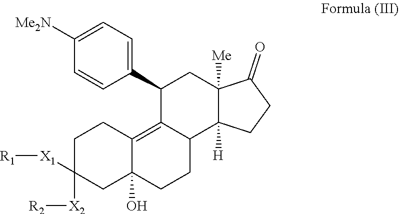

wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal.
254. The process of embodiment 253, wherein both X₁ and X₂ are O.
255. The process according to embodiments 253 or 254, wherein R₁ and R₂ are each independently optionally substituted C₁-C₆ alkyl.
256. The process of any one of embodiments 253-255, wherein R₁ and R₂ are each independently Me, Et, Pr, or Bu.
257. The process of any one of embodiments 253 or 254, wherein R₁, R₂, X₁, and X₂ are together with the carbon atom connected X₁ and X₂ to form optionally substituted 4-, 5-, 6-, 7-, or 8-membered cyclic ketal.
258. The process according to embodiments 253 or 254, wherein R₁, R₂, X₁, and X₂ are together with the carbon atom connected X₁ and X₂ to form optionally substituted 5- or 6-membered cyclic ketal.
259. The process according to embodiments 253 or 254, wherein R₁, R₂, X₁, and X₂ are together with the carbon atom connected X₁ and X₂ to form optionally substituted 5-membered cyclic ketal.
260. The process of embodiment 253, wherein the compound of Formula (II) has a formula of

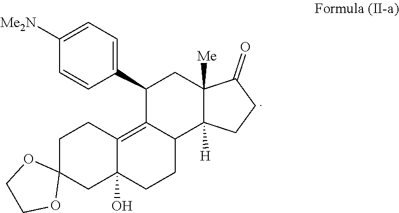 [SMILES: CN(C)c1ccc([C@H]2C[C@]3(C)C(=O)CC[C@H]3C3CC[C@@]4(O)CC5(CCC4=C32)OCCO5)cc1]

261. The process of embodiment 260, wherein the compound of Formula (III) has a formula of

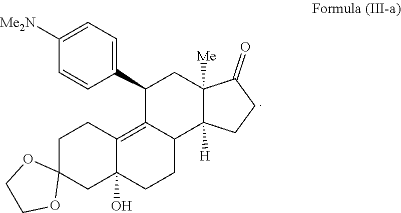 [SMILES: CN(C)c1ccc([C@H]2C[C@@]3(C)C(=O)CC[C@H]3C3CC[C@@]4(O)CC5(CCC4=C32)OCCO5)cc1]

262. The process of any one of embodiments 253-261, wherein the UV light is a narrow band frequency light at a wavelength from about 300 nm to about 320 nm.
263. The process of any one of embodiments 253-262, wherein the UV light is a narrow band frequency light with a wavelength at about 311 nm.
264. The process of any one of embodiments 253-263, wherein the UV light is from a low-pressure mercury lamp.
265. The process of any one of embodiments 253-264, wherein the low-pressure mercury lamp is fitted inside the flow reactor.
266. The process of any one of embodiments 253-265, wherein the flow reactor is fabricated from a long polytetrafluoroethylene (PTFE) tubing with an inner diameter between 1 to 20 millimeters.
267. The process of any one of embodiments 253-265, wherein the flow reactor is fabricated from a long fluorinated ethylene propylene (FEP) tubing with an inner diameter between 1 to 20 millimeters.
268. The process of any one of embodiments 253-265, wherein the flow reactor is fabricated from a long perfluoroalkoxy alkane (PFA) tubing with an inner diameter between 1 to 20 millimeters.
269. The process of any one of embodiments 253-268, wherein the organic solvent is ethyl acetate, toluene, methyltetrahydrofuran, tetrahydrofuran, isopropyl ether, methanol, water, dichloromethane, isopropylether, or a combination thereof.
270. The process of any one of embodiments 253-268, wherein the organic solvent is ethyl acetate.
271. The process of any one of embodiments 253-270 further comprising washing the solution with aqueous NaHSO₃ solution to remove the aldehyde impurities from the UV irradiation.
272. The process of any one of embodiments 253-271, further comprising purifying the crude compound of Formula (III) with solid absorbents such as silica gel, magnesium silicate, alumina, polymers, clays, or other porous or high surface area solids.
273. The process of any one of embodiments 253-272 further comprising recrystallizing the compound of Formula (III).
274. The process of embodiment 273, wherein the compound of Formula (III) is recrystallized from ethyl acetate and heptane.
275. The process of embodiment 274, wherein the volume ratio of ethyl acetate to heptane is about 1 to 3.
276. A process of preparing a compound of Formula (X), or a salt thereof, the process comprising:
   contacting the compound of

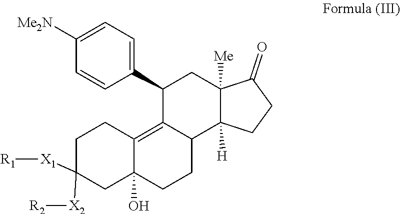

with an organometallic reagent having a formula of

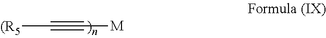

to produce the compound of

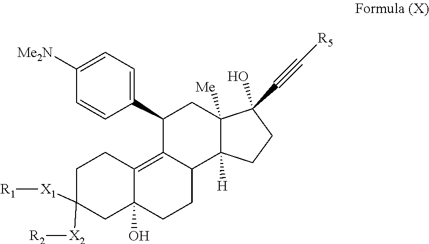

or a salt form thereof; and
wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and R₅ is trialkylsilyl or triakylsilyloxy.
277. The process of embodiment 276, wherein both X₁ and X₂ are O.
278. The process according to embodiments 276 or 277, wherein R₁ and R₂ are each independently optionally substituted C₁-C₆ alkyl.
279. The process of any one of embodiments 276-278, wherein R₁ and R₂ are each independently Me, Et, Pr, or Bu.
280. The process according to embodiments 276 or 277, wherein R₁, R₂, X₁, and X₂ are together with the carbon atom connected X₁ and X₂ to form optionally substituted 4-, 5-, 6-, 7-, or 8-membered cyclic ketal.
281. The process according to embodiments 276 or 277, wherein R₁, R₂, X₁, and X₂ are together with the carbon atom connected X₁ and X₂ to form optionally substituted 5- or 6-membered cyclic ketal.
282. The process according to embodiments 276 or 277, wherein R₁, R₂, X₁, and X₂ are together with the carbon atom connected X₁ and X₂ to form optionally substituted 5-membered cyclic ketal.
283. The process of any one of embodiments 276-282, wherein the organometallic reagent of Formula VIII is nucleophilic.
284. The process of embodiment 283, wherein the organometallic reagent of Formula VIII is an organolithium reagent.

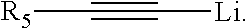

285. The process of embodiment 284, wherein the organolithium reagent is
286. The process of embodiment 285 further comprising preparing the organolithium reagent by contacting

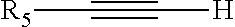

with an alkyllithium reagent in a first organic solvent at a low temperature.
287. The process of embodiment 286, wherein the alkyllithium reagent is n-butyllithum, n-heptanyllithoum or n-hexyllithium, or a combination thereof.
288. The process of embodiment 286, wherein the alkyllithium reagent is n-hexyllithium.
289. The process of embodiment 286, wherein the low temperature is below −40° C.
290. The process of embodiment 286, wherein the low temperature is about −70° C.
291. The process of any one of embodiments 283-290, wherein the compound of Formula (III) or a salt form thereof is separately dissolved in a second organic solvent before contacting the organolithium reagent at the low temperature.
292. The process of embodiment 291, wherein the compound of Formula (III) or a salt form thereof in the second organic solvent is added dropwise to the organolithium reagent in the first organic solvent at the low temperature.
293. The process of embodiment 292, wherein both the first and second organic solvents are polar non-protic organic solvents.
294. The process of embodiment 293, wherein both the first and second organic solvents are tetrahydrofuran or 2-methyltetrohydrafuran.
295. The process of embodiment 293, wherein both the first and second organic solvents are 2-methyltetrohydrafuran.
296. The process of any one of embodiments 283-295, wherein the molar ratio of the organolithium reagent to the compound of Formula (III) is between about 1:1 to about 10:1.
297. The process of any one of embodiments 283-295, wherein the molar ratio of the organolithium reagent to the compound of Formula (III) is about 4:1.
298. The process of any one of embodiments 283-297, wherein a diastereofacial selectivity for the compound of Formula (X) to the 17-β-hydroxy-epimer thereof is achieved.
299. The process of embodiment 298, wherein the diastereofacial selectivity of the compound of Formula (X) to the 17-β-hydroxy-epimer thereof, is more than about 1:1.
300. The process of embodiment 298, wherein the diastereofacial selectivity of the compound of Formula (X) to the 17-β-hydroxy-epimer thereof, is about 8:1.
301. The process of embodiment 298, wherein the diastereofacial selectivity of the compound of Formula (X) to the 17-β-hydroxy-epimer thereof, is about 13.4:1.
302. The process of any one of embodiments 283-301, further comprising isolating of the compound of Formula (X).
303. The process of embodiment 302, wherein the isolating of the compound of Formula (X) comprises:
   quenching the resulting mixture with ice water to form an aqueous phase and an organic phase;
   optionally washing the organic phase with water and brine;
   optionally drying the organic phase with a drying reagent; and
   evaporating the organic phase to form the compound of Formula (X).
304. The process of embodiment 303, wherein the drying reagent is anhydrous Na₂SO₄, MgSO₄, or CaSO₄.
305. The process of embodiment 303, wherein the drying reagent is anhydrous Na₂SO₄.
306. The process of any one of embodiments 276-305, the process further comprising: contacting the compound of Formula (X) in a third organic solvent with a fluoride-containing reagent to produce the compound having a formula of

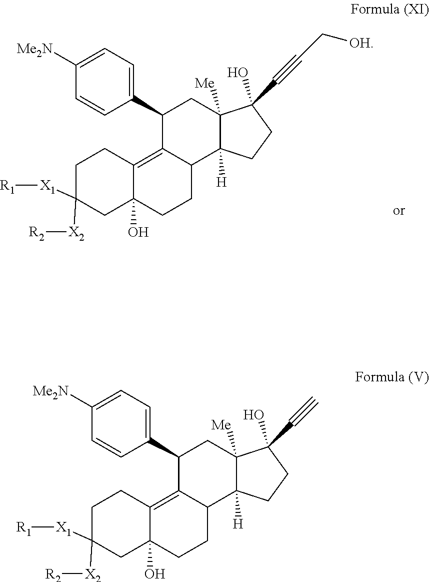

307. The process of embodiment 306, wherein the fluoride-containing reagent is tetra-n-butylammonium fluoride (TBAF), NH₄F, KH₂F, KF, NaF, hydrogen fluoride, hydrogen fluoride pyridine, tris(dimethylamino)sulfonium difluorotrimethylsilicate, triethylamine trihydrofluoride (Et₃N·3HF), tetrabutylammonium tetra(t-butyl alcohol)-coordinated fluoride (TBAF(tBuOH)₄), tetrabutylammonium triphenyldifluorosilicate, or a combination thereof.
308. The process of embodiment 306, wherein the fluoride-containing reagent is tetra-n-butylammonium fluoride (TBAF), NH₄F, KH₂F, KF, or a combination thereof.
309. The process of embodiment 306, wherein the fluoride-containing reagent is tetra-n-butylammonium fluoride (TBAF).
310. The process of embodiment 306, wherein the fluoride-containing reagent is NH₄F.
311. The process of embodiment 306, wherein the fluoride-containing reagent is KH₂F.
312. The process of embodiment 306, wherein the fluoride-containing reagent is KF.
313. The process of embodiment 306, wherein the fluoride-containing reagent is NaF.
314. The process of any one of embodiments 306-313, wherein the fluoride-containing reagent is added to the solution of the compound of Formula (X) in the third organic solvent to form a mixture.
315. The process of embodiment 314, wherein the mixture was optionally heated to a temperature and kept at the temperature for a period of time.
316. The process of embodiment 315, wherein the temperature is at least about 40° C.
317. The process of embodiment 315, wherein the temperature is about 70° C.
318. The process of embodiment 315, wherein the temperature is about 80° C.
319. The process of any one of embodiments 315-318, wherein the period of time is at least about 5 minutes.
320. The process of any one of embodiments 315-318, wherein the period of time is about 16 hours.
321. The process of any one of embodiments 306-320, wherein the third organic solvent is a polar non-protic organic solvent.
322. The process of embodiment 321, wherein the third organic solvent is dimethyl sulfoxide.
323. The process of any one of embodiments 315-322, wherein the heated mixture is cooled to room temperature to form a lower phase and an upper phase.
324. The process of embodiment 323, wherein the lower phase is optionally washed with a non-polar organic solvent.
325. The process of embodiment 324, wherein the non-polar organic solvent is one or more C₅-C₁₀ alkanes.
326. The process of embodiment 324, wherein the non-polar organic solvent is pentane, hexane, heptane, or a combination thereof.
327. The process of embodiment 326, wherein the non-polar organic solvent is heptane.
328. The process of any one of embodiments 323-327, wherein the lower phase is mixed with water to form a mixture and the mixture is extracted with a polar non-protic organic solvent.
329. The process of embodiment 328, wherein the mixture is extracted with the polar non-protic organic solvent at least once.
330. The process of embodiment 328, wherein the mixture is extracted with the polar non-protic organic solvent twice.
331. The process of any one of embodiments 328-330, wherein the polar non-protic organic solvent after extraction is optionally washed with water and brine, optionally dried over a drying reagent, and evaporated to form the compound having a formula of Formula (V) or Formula (XI) with the 17-β-hydroxy-epimer thereof as an impurity.
332. The process of embodiment 331, wherein the molar ratio of the compound of Formula (V) and the 17-β-hydroxy-epimer thereof is about 10:1.
333. The process of embodiment 331, wherein the molar ratio of the compound of Formula (XI) and the 17-β-hydroxy-epimer thereof is about 15.2:1.
334. The process of any one of embodiments 276-333, wherein R₅ is trialkylsilyl.
335. The process of any one of embodiments 276-334, wherein R₅ is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS).
336. The process of any one of embodiments 276-335, wherein R₅ is triisopropylsilyl (TIPS).
337. The process of any one of embodiments 306-336, wherein the process further comprising:
   contacting the compound of

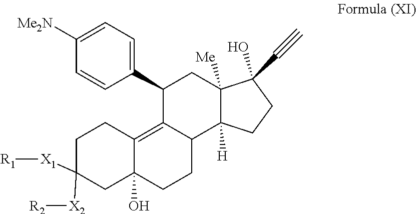

with paraformaldehyde, at least one bases, and CuI in a fourth organic solvent under suitable conditions to produce the compound of

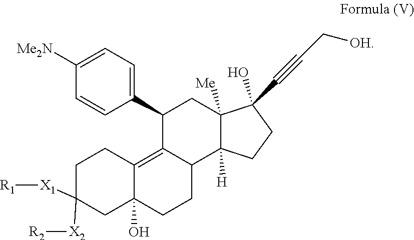

338. The process of embodiment 337, the process further comprising two bases.
339. The process of embodiment 338, wherein the two bases are a trialkylamine and an alkali hydroxide.
340. The process of embodiment 339, wherein the trialkylamine is triethylamine.
341. The process according to embodiments 338 or 339, wherein the alkali hydroxide is KOH.
342. The process of embodiment 337, wherein the fourth organic solvent is a non-protic organic solvent.
343. The process of embodiment 342, wherein the non-protic organic solvent is a polar non-protic organic solvent.
344. The process of embodiment 343, wherein the polar non-protic organic solvent is dimethyl sulfoxide.
345. The process of any one of embodiments 337-344, wherein the diastereofacial selectivity of the compound of Formula (V) to the 17-β-hydroxy-epimer thereof is at least about 10:1.
346. The process of any one of embodiments 337-344, wherein the diastereofacial selectivity of the compound of Formula (V) to the 17-β-hydroxy-epimer thereof is about 15.3:1.
347. The process of any one of embodiments 276-346, wherein the compound of Formula (III) has a formula of

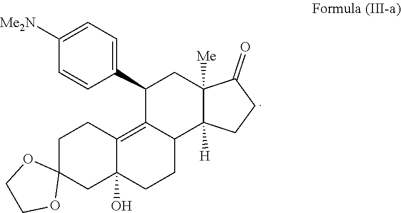 [SMILES: CN(C)c1ccc([C@H]2C[C@@]3(C)C(=O)CC[C@H]3C3CC[C@@]4(O)CC5(CCC4=C32)OCCO5)cc1]

348. The process of embodiment 347, wherein the compound of Formula (X) has a formula of

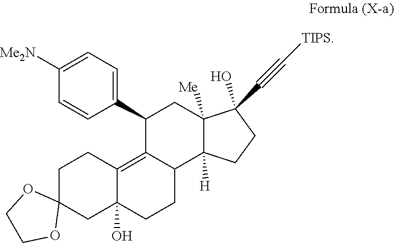

349. The process of any one of embodiments 306-348, wherein the compound of Formula (XI) has a formula of

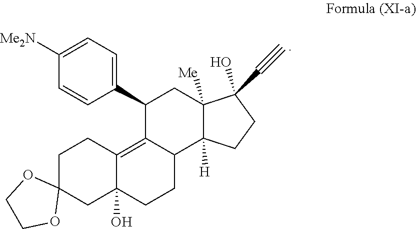 [SMILES: C#C[C@@]1(O)CC[C@H]2C3CC[C@@]4(O)CC5(CCC4=C3[C@@H](c3ccc(N(C)C)cc3)C[C@]21C)OCCO5]

350. The process of any one of embodiments 276-333, wherein the compound of Formula (X) or a salt form thereof, has a formula of

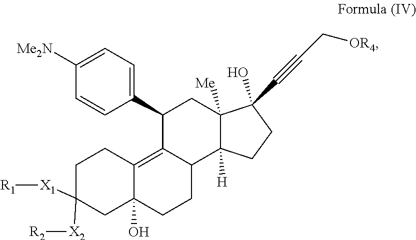

wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₄ is trialkylsilyl.
351. The process of embodiment 350, wherein Ra is trimethylsilyl (TMS), triethylsilyl (TES), tert-butyldimethylsilyl (TBS), tert-butyldiphenylsilyl (TBDPS), thexyldimethylsilyl, or triisopropylsilyl (TIPS).
352. The process of embodiment 350, wherein R⁴ is triisopropylsilyl (TIPS).
353. The process of any one of embodiments 350-352, wherein the compound of Formula (III) has a formula of

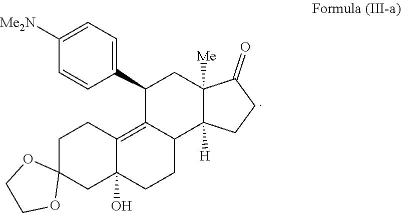 [SMILES: CN(C)c1ccc([C@H]2C[C@@]3(C)C(=O)CC[C@H]3C3CC[C@@]4(O)CC5(CCC4=C32)OCCO5)cc1]

354. The process of embodiment 353, wherein the compound of Formula (IV) has a formula of

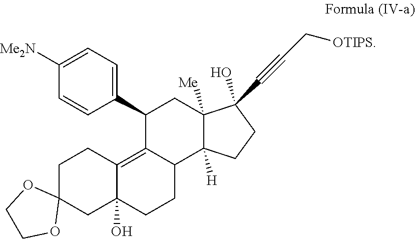

355. The process of any one of embodiments 306-333 and 350-354, wherein the compound of Formula (V) has a formula of

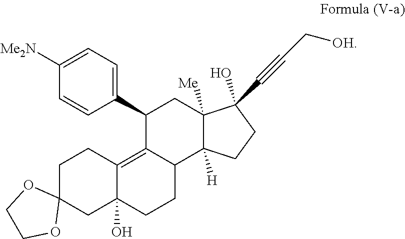 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

356. The process of any one of embodiments 306-355, further comprising purifying the compound of Formula (V) from the 17-β-hydroxy-epimer thereof comprising forming an aggregate of the compound of Formula (V) with a salt.
357. The process of embodiment 356, wherein the salt is a citrate salt or an acetate salt.
358. The process of embodiment 356, wherein the salt is a citrate salt.
359. The process of embodiment 358, wherein the citrate salt is lithium citrate.
360. The process of embodiment 356, wherein the salt is an acetate salt.
361. The process of embodiment 360, wherein the acetate salt is potassium acetate.
362. The process of any one of embodiments 356-361, wherein the molar ratio of the compound of Formula (V) to the 17-β-hydroxy-epimer thereof in the aggregate is more than about 20:1, about 40:1, about 60:1, about 80:1, about 100:1, about 150:1, about 200:1, about 250:1, about 300:1, about 350:1, or about 400:1.
363. The process of any one of embodiments 356-362, wherein the aggregate is formed in a mixture of the polar non-protic organic solvent and the non-polar organic solvent.
364. The process of embodiment 363, wherein the volume ratio of the polar non-protic organic solvent to the non-polar organic solvent in the mixture is about 4.4:1.
365. The process of any one of embodiments 363 or 364, wherein the polar non-protic organic solvent is ethyl acetate.
366. The process of any one of embodiments 363-365, wherein the non-polar organic solvent is heptane.
367. The process of any one of embodiments 356-366, further comprising dissolving the aggregate in the polar non-protic organic solvent to form an organic phase, washing the organic phase with water, and concentrating the organic phase to form the compound of Formula (V) in high purify.
368. The process of embodiment 367, wherein the purity of the compound of Formula (V) is at least about 90%, at least about 92%, at least about 94%, at least about 96%, at least about 98%, or at least about 99%.
369. A process of preparing a compound of Formula (VII), or a pharmaceutically acceptable salt thereof, the process comprising:
   a) subjecting the compound of

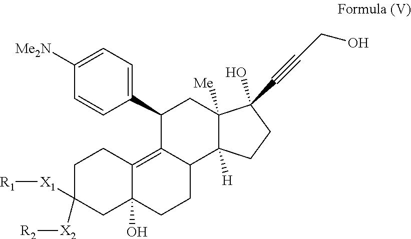

or a salt form thereof, prepared according to any process of embodiments 306-368 under a hydrogenation condition to produce the compound of

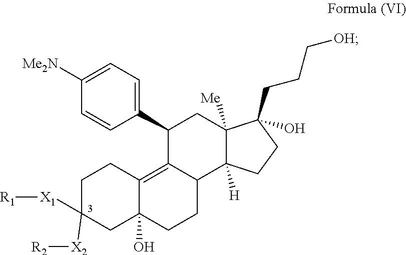

and
e) hydrolyzing the compound of Formula (VI) under a hydrolysis condition to produce the compound of

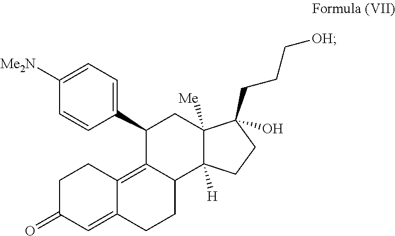 [SMILES: CN(C)c1ccc([C@H]2C[C@]3(C)[C@@H](CC[C@]3(O)CCCO)C3CCC4=CC(=O)CCC4=C32)cc1]

or a pharmaceutically acceptable salt
370. The process of embodiment 369, wherein the hydrogenation condition is suitable for converting an unsaturated carbon-carbon triple bond to a saturated carbon-carbon bond.
371. The process of embodiment 370, wherein the suitable hydrogenation condition comprises hydrogen and a metal catalyst.
372. The process of embodiment 371, wherein the metal catalyst comprises palladium.
373. The process of embodiment 369, wherein the hydrolysis condition is a suitable condition for removing the protection for the 3-ketone of Formula (VI).
374. The process of embodiment 373, wherein the suitable hydrolysis condition comprises sulfuric acid.
375. The process of any one of embodiments 369-374, further comprising recrystallizing the compound of Formula (VII).
376. The process of embodiment 375, wherein the compound of Formula (VII) is recrystallized from a mixture of at least one polar solvent and/or at least one non-polar solvent
377. The process of embodiment 375, wherein the compound of Formula (VII) is recrystallized from a mixture of at least one polar solvent and at least one non-polar solvent
378. The process of embodiment 375, wherein the compound of Formula (VII) is recrystallized from a mixture of one polar solvent and one non-polar solvent
379. The process of any one of embodiments 376-378, wherein the polar solvent is ethyl acetate.
380. The process of any one of embodiments 376-379, wherein the polar solvent is n-heptane.
381. The process of embodiment 376, wherein the mixture is a mixture of ethyl acetate and n-heptane.
382. The process of embodiment 381, wherein the volume ratio of ethyl acetate to n-heptane is about 1 to 3.
383. A process of preparing a compound of Formula (XII), or a pharmaceutically acceptable salt thereof, the process comprising:
   subjecting the compound of

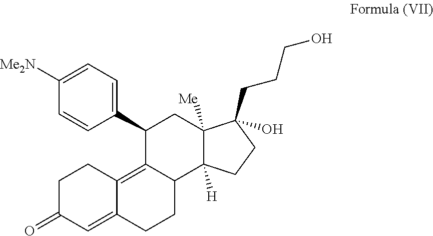 [SMILES: CN(C)c1ccc([C@H]2C[C@]3(C)[C@@H](CC[C@]3(O)CCCO)C3CCC4=CC(=O)CCC4=C32)cc1]

or pharmaceutically acceptable salt thereof, prepared according to any process of embodiments 157-170 under a suitable N-demethylation condition to produce the compound of

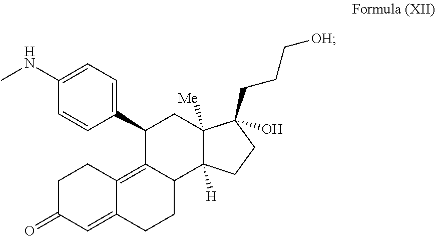 [SMILES: CNc1ccc([C@H]2C[C@]3(C)[C@@H](CC[C@]3(O)CCCO)C3CCC4=CC(=O)CCC4=C32)cc1]

or a pharmaceutically acceptable salt.
384. The process of embodiment 383, wherein the suitable N-demethylation condition is suitable for removing a methyl group from the dimethylamino phenyl group on the compound of Formula (VII).
385. The process according embodiments 383 or 384, wherein the suitable N-demethylation condition is a von Braun reaction, a method of applying chloroformate, a Polonovski reaction, a photochemical method, a microbial method, or an enzymatic method.
386. The process of embodiment 385, wherein the enzymatic method is a cytochrome P450 enzyme mediated N-demethylation.
387. The process of embodiment 385, wherein the enzymatic method is a cytochrome P450 3A-mediated N-demethylation.
388. A compound having a formula of

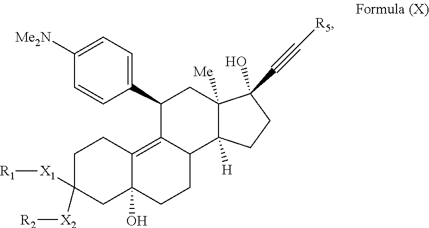

or a pharmaceutically acceptable salt thereof,
wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₅ is trialkylsilyl or triakylsilyloxy.
389. The compound of embodiment 388, or a pharmaceutically acceptable salt thereof, wherein both X₁ and X₂ are O.
390. The compound according to embodiments 388 or 389, or a pharmaceutically acceptable salt thereof, wherein R¹ and R₂ are each independently optionally substituted C₁-C₆ alkyl.
391. The compound of any one of embodiments 388-390, or a pharmaceutically acceptable salt thereof, wherein R¹ and R₂ are each independently Me, Et, Pr, or Bu.
392. The compound of any one of embodiments 388-390, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
393. The compound of any one of embodiments 388-390, or a pharmaceutically acceptable salt thereof, wherein R₁, R₂, X₁, and X₂ are together with the carbon atom connected X₁ and X₂ to form optionally substituted 5- or 6-membered cyclic ketal.
394. The compound of any one of embodiments 388-390, or a pharmaceutically acceptable salt thereof, wherein R₁, R₂, X₁, and X₂ are together with the carbon atom connected X₁ and X₂ to form optionally substituted 5-membered cyclic ketal.
395. The compound of any one of embodiments 388-394, wherein R₅ is trialkylsilyl.
396. The compound of any one of embodiments 388-394, wherein R₅ is trimethylsilyl (TMS), triethylsilyl (TES), tert-butyldimethylsilyl (TBS), tert-butyldiphenylsilyl (TBDPS), or triisopropylsilyl (TIPS).
397. The compound of any one of embodiments 388-394, wherein R₅ is triisopropylsilyl (TIPS).
398. The compound of embodiment 388, or a pharmaceutically acceptable salt thereof, wherein the compound of Formula (X) has a formula of

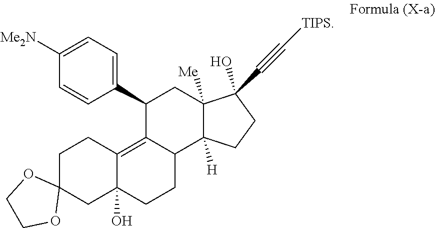

399. The compound of any one of embodiments 388-394, wherein R₅ is triakylsilyloxy.
400. The compound of any one of embodiments 388-394, wherein R₅ is trimethylsilyloxy (OTMS), triethylsilyloxy (OTES), tert-butyldimethylsilyloxy (OTBS), tert-butyldiphenylsilyloxy (OTBDPS), or triisopropylsilyloxy (OTIPS).
401. The compound of any one of embodiments 388-394, wherein R₅ is triisopropylsilyloxy (OTIPS).
402. The compound of embodiment 388, or a pharmaceutically acceptable salt thereof, wherein the compound of Formula (X) has a formula of

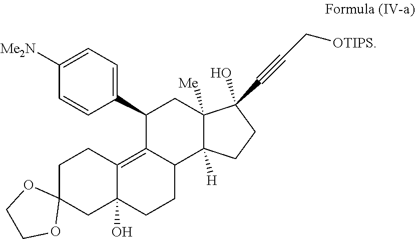

403. A compound having a formula of

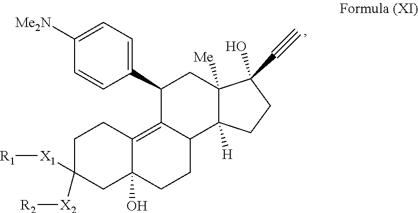

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
404. The compound of embodiment 403, or a pharmaceutically acceptable salt thereof, wherein both X₁ and X₂ are O.
405. The compound according to embodiments 403 or 404, or a pharmaceutically acceptable salt thereof, wherein R¹ and R₂ are each independently optionally substituted C₁-C₆ alkyl.
406. The compound of any one of embodiments 403-405, or a pharmaceutically acceptable salt thereof, wherein R¹ and R₂ are each independently Me, Et, Pr, or Bu.
407. The compound according to embodiments 403 or 404, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
408. The compound according to embodiments 403 or 404, or a pharmaceutically acceptable salt thereof, wherein R₁, R₂, X₁, and X₂ are together with the carbon atom connected X₁ and X₂ to form optionally substituted 5-membered cyclic ketal.
409. The compound of embodiment 403, or a pharmaceutically acceptable salt thereof, wherein the compound of Formula (XI) has a formula of

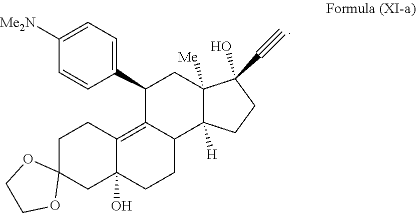 [SMILES: C#C[C@@]1(O)CC[C@H]2C3CC[C@@]4(O)CC5(CCC4=C3[C@@H](c3ccc(N(C)C)cc3)C[C@]21C)OCCO5]

410. A compound having a formula of

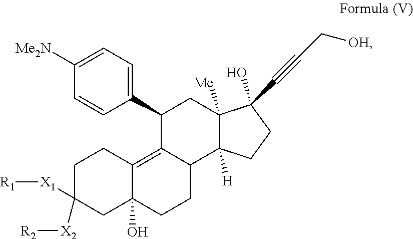

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
411. The compound of embodiment 410, or a pharmaceutically acceptable salt thereof, wherein both X₁ and X₂ are O.
412. The compound according to embodiments 410 or 411, or a pharmaceutically acceptable salt thereof, wherein R¹ and R₂ are each independently optionally substituted C₁-C₆ alkyl.
413. The compound of any one of embodiments 410-412, or a pharmaceutically acceptable salt thereof, wherein R¹ and R₂ are each independently Me, Et, Pr, or Bu.
414. The compound according to embodiments 410 or 411, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
415. The compound according to embodiments 410 or 411, or a pharmaceutically acceptable salt thereof, wherein R₁, R₂, X₁, and X₂ are together with the carbon atom connected X₁ and X₂ to form optionally substituted 5-membered cyclic ketal.
416. The compound of embodiment 410, or a pharmaceutically acceptable salt thereof, wherein the compound of Formula (V) has a formula of

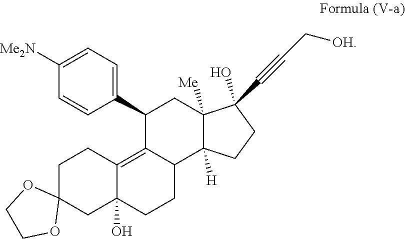 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

417. A compound having a formula of

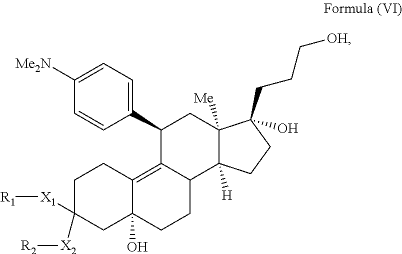

or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.
418. The compound of embodiment 417, or a pharmaceutically acceptable salt thereof, wherein both X₁ and X₂ are O.
419. The compound according to embodiments 417 or 418, or a pharmaceutically acceptable salt thereof, wherein R¹ and R₂ are each independently optionally substituted C₁-C₆ alkyl.
420. The compound of any one of embodiments 417-419, or a pharmaceutically acceptable salt thereof, wherein R¹ and R₂ are each independently Me, Et, Pr, or Bu.
421. The compound according to embodiments 417 or 418, or a pharmaceutically acceptable salt thereof, wherein R₁, R₂, X₁, and X₂ are together with the carbon atom connected X₁ and X₂ to form optionally substituted 4-, 5-, 6-, 7-, or 8-membered cyclic ketal.
422. The compound according to embodiments 417 or 418, or a pharmaceutically acceptable salt thereof, wherein R₁, R₂, X₁, and X₂ are together with the carbon atom connected X₁ and X₂ to form optionally substituted 5-membered cyclic ketal.
423. The compound of embodiment 417, or a pharmaceutically acceptable salt thereof,
   wherein the compound of Formula (VI) has a formula of

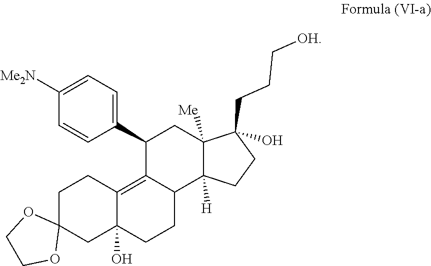 [SMILES: CN(C)c1ccc([C@H]2C[C@]3(C)[C@@H](CC[C@]3(O)CCCO)C3CC[C@@]4(O)CC5(CCC4=C32)OCCO5)cc1]

In order that the embodiments disclosed herein may be more efficiently understood, examples are provided below. It should be understood that these examples are for illustrative purposes only and are not to be construed as limiting the embodiments in any manner.

The following examples are illustrative, but not limiting, of the processes described herein. Other suitable modifications and adaptations of the variety of conditions and parameters normally encountered in therapy, synthesis, and other embodiments disclosed herein are within the spirit and scope of the embodiments.

### EXAMPLES

Certain synthetic schemes, both general and specific, are provided herein. The compounds disclosed herein can be made according to the processes described herein or intermediates that lead to the compounds disclosed herein can be made according to the processes described herein. The substitutions can be varied according to the compound or intermediate being made based upon the following examples and other modifications known to one of skill in the art.

The processes disclosed herein were used to prepare the following compounds in the following examples or the examples were varied according to one of skill in the art to prepare the compounds.

### Example 1: Processes of Preparing Compounds of Formula (II)

### General Procedure A:

Some compounds of Formula (II) can be prepared via a base-catalyzed fragmentation of compounds of Formula (I) as shown by the methods outlined in Scheme 1. A compound of Formula (I) was treated with a suitable base (e.g., t-BuOK) under suitable conditions that can yield the corresponding compound of Formula (II). The variables in Scheme 1 are as defined in the embodiments as described herein.

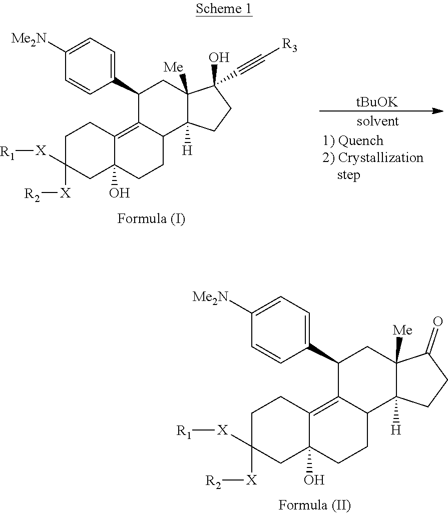

### Example 1a: Synthesis of the Compound of Formula (II-a), (5R,11R,13S,14S)-11-(4-(dimethylamino)phenyl)-5-hydroxy-13-methyl-1,4,5,6,7,8,11,12,13,14,15,16-dodecahydrospiro[cyclopenta[a]phenanthrene-3,2′-[1,3]dioxolan]-17 (2H)-one

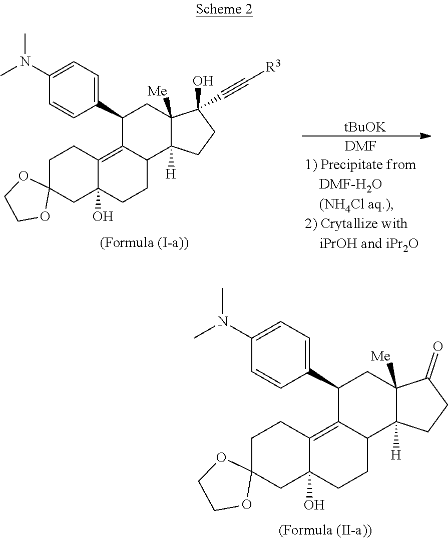

A mixture of tBuOK (22.8 g, 0.20 mol, 0.1 eq) and DMF (4 L) was added to a solution of the compound of Formula (I-a) (1 kg, 2.04 mol, 1.0 eq) in DMF (500 mL) in about 1 h at 70° C. Thereafter, the reaction mixture was stirred at 70° C. for about 0.5 h before being cooled to room temperature. To the cooled reaction mixture was added 7.5% wt NH₄Cl (20 L) aqueous in 1 h to form a suspension. The suspension was filtered to form a filter cake and the filter cake was rinsed with water (5 L×2) and dried under atmospheric pressure at about 50° C. to afford crude solids. The crude solids were crystallized with a mixture solvent of iPrOH (2.75 L) and iPr₂O (2.75 L) to afford the compound Formula (II-a) (739 g, 81% yield) as off-white solids.

¹H NMR (400 MHZ, CDCl3) δ 7.06 (d, J=8.6 Hz, 2H), 6.64 (d, J=8.8 Hz, 2H), 4.38 (s, 1H), 4.24 (d. J=6.9 Hz, 1H), 4.07-3.87 (m, 4H), 2.91 (s, 6H), 2.51-2.26 (m, 5H), 2.07-1.96 (m, 4H), 1.87-1.74 (m, 3H), 1.67-1.49 (m, 6H), 1.37-1.22 (m, 2H), 0.51 (s, 3H).

### Example 1b: Synthesis of the Compound of Formula (II-a), (5R,11R,13S,14S)-11-(4-(dimethylamino)phenyl)-5-hydroxy-13-methyl-1,4,5,6,7,8,11,12,13,14,15,16-dodecahydrospiro[cyclopenta[a]phenanthrene-3,2′-[1,3]dioxolan]-17 (2H)-one

A solution of tBuOK (114 mg, 1.02 mmol, 1.0 eq) and tBuOH (0.5 mL) was added to a solution of the compound of Formula (I-a) (0.5 g, 1.02 mmol, 1.0 eq) and tBuOH (5 mL) in about 0.5 h at about 70° C. Thereafter, the reaction was stirred for about 6 h before being cooled to room temperature and added to water (10 mL) in 10 min, which was extracted with ethyl acetate (10 mL×3). The organic phases were combined and washed with brine (10 mL) before solvent being removed under vacuum to afford 490 mg the compound of Formula (II-a), (93.7% yield; 95.9% purity).

¹H NMR (400 MHZ, CDCl3) δ 7.06 (d, J=8.6 Hz, 2H), 6.64 (d, J=8.8 Hz, 2H), 4.38 (s, 1H), 4.24 (d. J=6.9 Hz, 1H), 4.07-3.87 (m, 4H), 2.91 (s, 6H), 2.51-2.26 (m, 5H), 2.07-1.96 (m, 4H), 1.87-1.74 (m, 3H), 1.67-1.49 (m, 6H), 1.37-1.22 (m, 2H), 0.51 (s, 3H).

### Example 1c: Synthesis of the Compound of Formula (II-a), (5R,11R,13S,14S)-11-(4-(dimethylamino)phenyl)-5-hydroxy-13-methyl-1,4,5,6,7,8,11,12,13,14,15,16-dodecahydrospiro[cyclopenta[a]phenanthrene-3,2′-[1,3]dioxolan]-17 (2H)-one

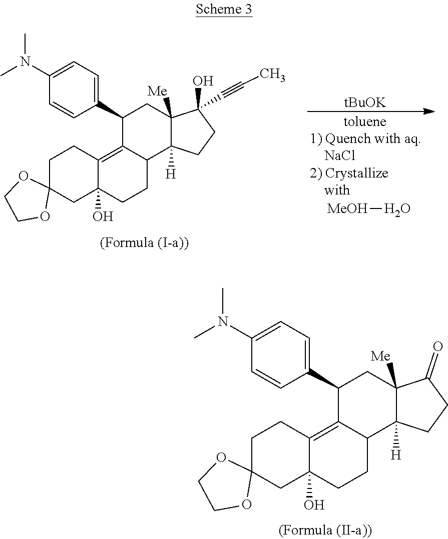

tBuOK (1.5 kg, 13.3 mmol, 0.131 eq) was added to a solution of the compound of Formula (I-a) (50 kg, 102 mmol, 1.0 eq) and toluene (300 L) in about 0.5 h at about 55-60° C. Thereafter, the reaction was stirred for about 0.5 h to drive the reaction to completion. Then 20% wt aqueous NaCl solution (250 L) was added into the reaction mixture, and the batch temperature was cooled to 10-30° C. The organic phase was separated and further washed with 20% wt aqueous NaCl twice (250 L each time). After aqueous NaCl solution wash, the organic phase was concentrated under vacuum between 60-70° C. until no continuous drops could be observed. Then methanol (300 L) was added in two portions and concentrated under vacuum between 60-70° C. until no continuous drops could be observed. Additional methanol (150 L) was added to the residue and stirred between 50-55° C. for 10-20 minutes. Then water (500 L) was added to crystallize, where precipitations were filtered through a centrifuge to afford the crude product. The crude product was further dried under vacuum between 50-80° C. to afford 41.3 kg the compound of Formula (II-a), (89.9% yield; 92.6% purity). 1H NMR (400 MHZ, CDCl3) δ 7.06 (d, J=8.6 Hz, 2H), 6.64 (d, J=8.8 Hz, 2H), 4.38 (s, 1H), 4.24 (d, J=6.9 Hz, 1H), 4.07-3.87 (m, 4H), 2.91 (s, 6H), 2.51-2.26 (m, 5H), 2.07-1.96 (m, 4H), 1.87-1.74 (m, 3H), 1.67-1.49 (m, 6H), 1.37-1.22 (m, 2H), 0.51 (s, 3H).

### Example 2: Processes of Preparing Compounds of Formula (III)

### General Procedure B:

Some compounds of Formula (III) can be prepared via stereoisomerism at carbon 13 by free radical C13-C17 ring-opening and reclosure of compounds of Formula (II) as shown by the methods outlined in Schemes 4 and 5. A compound of Formula (II) was subjected to a continuous photochemical isomerization using FEP (fluorinated ethylene propylene) or, alternatively, PFA (perfluoroalkoxy alkane) or PTFE (polytetrafluoroethylene) tubular reactors, all of which have the advantage of being resistant to fouling from sticky film formation, permeable to UV light, and having an adjustable and extended residence time, safer operating conditions, and being readily cleaned and replaced. An unexpected and surprising improvement over the prior art is that the aldehyde impurities generated from the photochemical reaction could be removed by washing with aqueous bisulfite solution selectively, despite having the same molecular weight as the desired product. The variables in Schemes 4 and 5 are as defined in the embodiments as described herein.

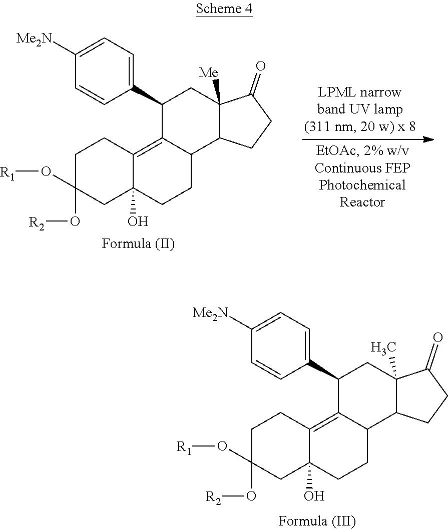

### Example 2a: Synthesis of the Compound of Formula (III-a), (5R,11R,13R)-11-(4-(dimethylamino)phenyl)-5-hydroxy-13-methyl-1,4,5,6,7,8,11,12,13,14,15,16-dodecahydrospiro[cyclopenta[a]phenanthrene-3,2′-[1,3]dioxolan]-17 (2H)-one

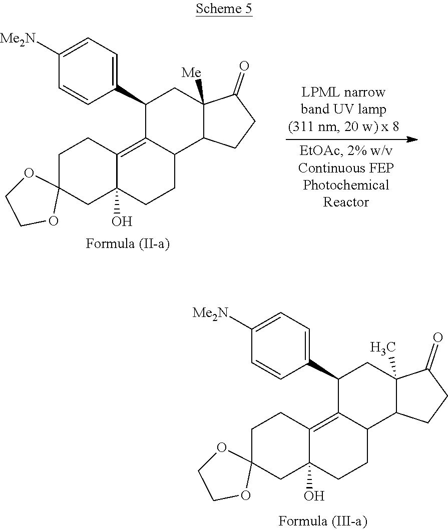 [SMILES: CN(C)c1ccc([C@H]2C[C@]3(C)C(=O)CCC3C3CC[C@@]4(O)CC5(CCC4=C32)OCCO5)cc1 ; CN(C)c1ccc([C@H]2C[C@@]3(C)C(=O)CCC3C3CC[C@@]4(O)CC5(CCC4=C32)OCCO5)cc1]

A solution of the compound of Formula (II-a) (1.2 kg, 2.66 mol) and EtOAc (60 L) was irradiated through a flow reactor (FEP tubing length 192 m, inner diameter 1.575 mm, outer diameter 3.175 mm) fitted with a low-pressure mercury lamp (a narrow band UV lamp, 311 nm wavelength, 8×20 w) inside the reactor. After the ¹HNMR analysis showed that there was ≤3.0% of the starting material remaining, the volume of the reaction mixture was reduced to 30 L via evaporation and the reaction mixture was then washed with a mixture of NaHSO₃ solution (20% wt, 30 L) and pyridine (1.5 L) before being washed with NaCl solution (20%, 30 L). Then solvent of the reaction mixture was concentrated under a vacuum below 45° C. to produce a residue that was then dissolved in toluene (1.473 L) and filtered through a plug of silica gel to (5× silica, 40 volumes of 50% EtOAc in heptane as eluent). The combined fractions containing the compound of Formula (III-a) from the silica-gel plug purification were concentrated under vacuum below 45° C. to produce crude solids of the compound of Formula (III-a) that was recrystallized from EtOAc (0.57 L, 1V) and heptane (1.71 L, 3 V) to afford the compound of Formula (III-a) (446.1 g, 36.4% yield) as off-white solids. The mother liquor resulted from the recrystallization was concentrated under vacuum below 45° C. to give a yellow foam residue that was dissolved in toluene (280 mL) subject to purification through silica gel chromatography (5× silica, 40V 50% EtOAc in heptane as eluent). The combined fractions containing the compound of Formula (III-a) from the silica-gel chromatography were removed under vacuum below 45° C. and recrystallized from EtOAc (106 mL, 1V) and heptane (318 mL, 3V) to afford an additional compound of Formula (III-a) (35.2 g, 2.9% yield) as off-white solids. the compound of Formula (III-a) was obtained in a total amount of 481.3 g; 39.3%.

¹H NMR (400 MHZ, CDCl₃) δ 6.96 (d, J=8.6 Hz, 2H), 6.63 (d, J=8.7 Hz, 2H), 4.34 (s, 1H), 4.02-3.83 (m, 4H), 3.75 (dd, J=11.6, 6.5 Hz, 1H), 2.90 (d, J=6.2 Hz, 6H), 2.37-2.14 (m, 6H), 1.94-1.75 (m, 6H), 1.66-1.46 (m, 6H), 1.11 (s, 3H), 1.07-0.97 (m, 1H).

### Example 2b: Synthesis of the Compound of Formula (III-a), (5R,11R,13R)-11-(4-(dimethylamino)phenyl)-5-hydroxy-13-methyl-1,4,5,6,7,8,11,12,13,14,15,16-dodecahydrospiro[cyclopenta[a]phenanthrene-3,2′-[1,3]dioxolan]-17 (2H)-one

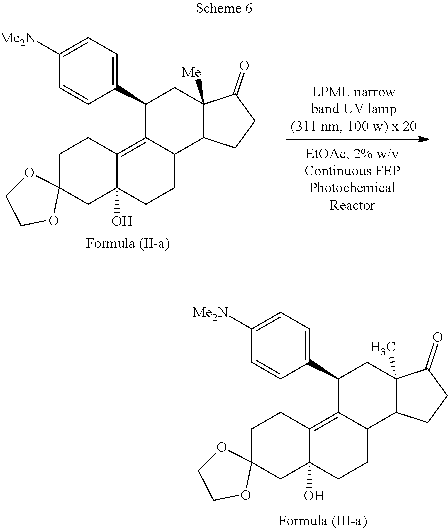 [SMILES: CN(C)c1ccc([C@H]2C[C@]3(C)C(=O)CCC3C3CC[C@@]4(O)CC5(CCC4=C32)OCCO5)cc1 ; CN(C)c1ccc([C@H]2C[C@@]3(C)C(=O)CCC3C3CC[C@@]4(O)CC5(CCC4=C32)OCCO5)cc1]

A solution of the compound of Formula (II-a) (0.458 g, 0.932 mol) and EtOAc (22.9 L) was irradiated through a flow reactor (FEP tubing length 300 m, inner diameter 4.35 mm, outer diameter 6.35 mm) fitted with low-pressure mercury lamps (narrow band UV lamps, 311 nm wavelength, 28×100 w) inside the reactor. After HPLC analysis showed that there was ≤4.0% of the starting material remaining, the volume of the reaction mixture was reduced to 10 L via evaporation and the reaction mixture was then washed with a mixture of NaHSO₃ solution (10% wt, 10 L) and NaHCO₃ (183 g) before being washed with saturated NaCl solution (9.16 L). Then solvent of the organic phase was concentrated under a vacuum below 45° C. to produce a residue that was then dissolved in toluene (458 mL) and filtered through a plug of silica gel to (5× silica, 40-50 volumes of 50% EtOAc in petroleum ether as eluent, the eluent also contains 0.5% triethylamine). The combined fractions containing the compound of Formula (III-a) from the silica-gel plug purification were concentrated under vacuum below 45° C. to produce crude solids of the compound of Formula (III-a) that was recrystallized from EtOAc (0.11 L) and heptane (0.32 L) to afford the compound of Formula (III-a) (188.6 g, 41.2% yield) as off-white solids.

¹H NMR (400 MHZ, CDCl₃) δ 6.96 (d, J=8.6 Hz, 2H), 6.63 (d, J=8.7 Hz, 2H), 4.34 (s, 1H), 4.02-3.83 (m, 4H), 3.75 (dd, J=11.6, 6.5 Hz, 1H), 2.90 (d, J=6.2 Hz, 6H), 2.37-2.14 (m, 6H), 1.94-1.75 (m, 6H), 1.66-1.46 (m, 6H), 1.11 (s, 3H), 1.07-0.97 (m, 1H).

### Example 3: Processes of Preparing Compounds of Formula (V) Via O-Trialkylsilyl-Propynol Route

### General Procedure C:

Some compounds of Formula (V) can be prepared via alkylation with an organometallic reagent having a formula of Formula (IX) as described or provided herein to form intermediates of Formula (IV), which was subject to desilylation with a fluoride-containing reagent as described or provided herein to form crude compounds of Formula (V) by the methods outlined in Scheme 6. The crude compounds of Formula (V) were purified by forming an aggregate with a suitable salt as described or provided herein and the aggregate was further crystallized in the suitable solvent to produce compounds of Formula (V) in higher purity and diastereoselectivity. The pure compounds of Formula (V) were readily freed from the aggregates by partitioning the aggregates in polar organic solvent and water as described or provided herein. The variables in Scheme 6 are as defined in the embodiments as described herein.

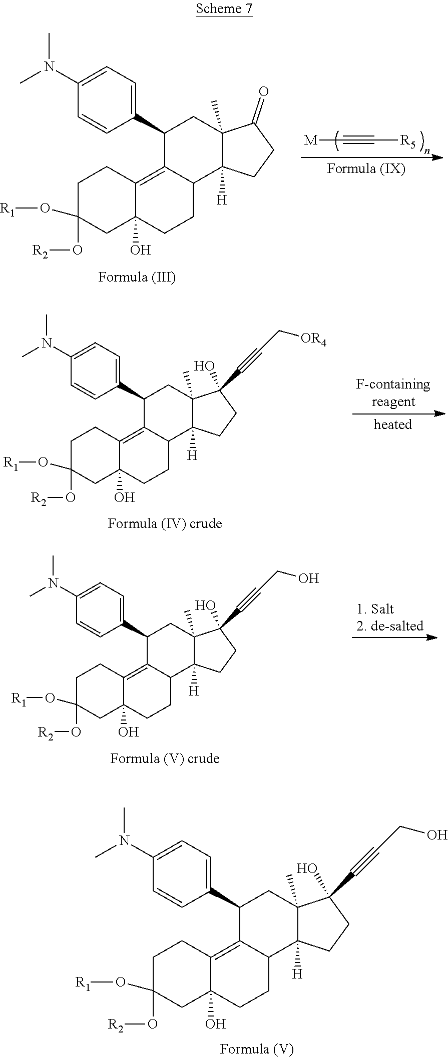

### Example 3a: Synthesis of Compound of Formula (V-a), (5R,11R,13R,14S,17R)-11-(4-(dimethylamino)phenyl)-17-(3-hydroxyprop-1-yn-1-yl)-13-methyl-1,2,6,7,8,11,12,13,14,15,16,17-dodecahydrospiro[cyclopenta[a]phenanthrene-3,2′-[1,3]dioxolane]-5,17(4H)-diol

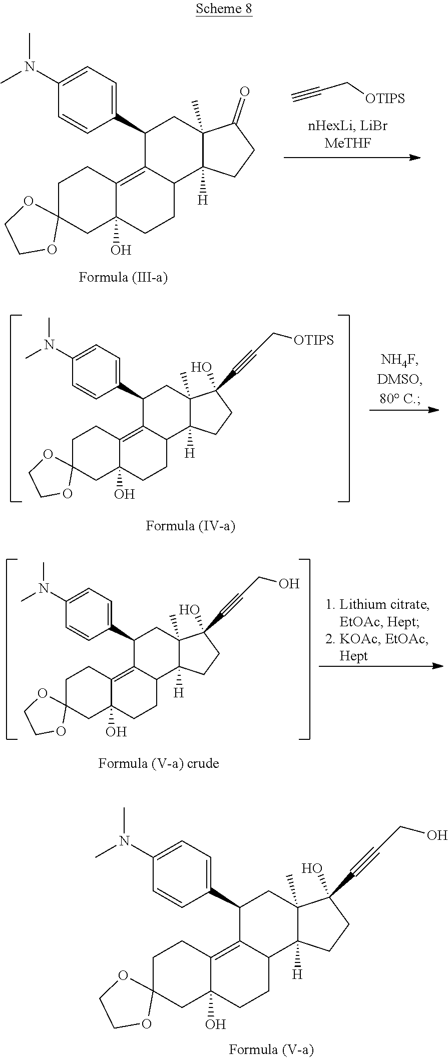

Preparation of the compound of Formula (IV-a), (5R,11R,13R,14S,17R)-11-(4-(dimethylamino)phenyl)-13-methyl-17-(3-((triisopropylsilyl)oxy)prop-1-yn-1-yl)-1,2,6,7,8,11,12,13,14,15,16,17-dodecahydrospiro[cyclopenta[a]phenanthrene-3,2′-[1,3]dioxolane]-5,17(4H)-diol.

A solution of O-triisopropylsilyl propargyl alcohol (847 g, 3.99 mol), LiBr (346 g, 3.99 mol) and MeTHF (2.25 L) was cooled to about-70° C. To the cooled solution, was dropwise added nHexLi solution (2.2 mol/L in hexane, 1.81 L, 3.99 mol) over about 2.5 h to form a mixture. To the mixture, was dropwise added a solution of the compound of Formula (III-a) (2.25 L MeTHF, 450 g of the compound of Formula (III-a), 1.0 mol) over about 5 h. After addition of the compound of Formula (III-a) solution, the reaction mixture was warmed to about 10° C. and stirred at that temperature for about 16 h. HPLC analysis showed that there were 10% the compound of Formula (III-a) remaining, 77% the compound of Formula (IV-a) formed, and 11% 17-β-hydroxy-epimer detected. The reaction mixture was then quenched and washed by ice water (4.5 L). The organic phase was then washed with water (4.5 L), brine (4.5 L), and dried with anhydrous Na₂SO₄ and, then, was evaporated under vacuum below 45° C. to afford 1410.9 g crude the compound of Formula (IV-a).

### Preparation of Crude the Compound of Formula (V-a)

The crude compound of Formula (IV-a) and NH₄F (160 g, 4.33 mol) were dissolved in DMSO (2.4 L) to form a solution and the solution was heated to about 70° C. and stirred for about 16 h. After HPLC analysis shown that the remaining compound of Formula (IV-a) is equal or less than 1%, the reaction mixture was cooled to room temperature to form an upper phase and a lower phase. The upper phase was removed, while the lower phase was extracted with heptane (1.2 L). Then water (2.4 L) was added to the lower phase and extracted with EtOAc (2×2.4 L). The organic phases were combined and washed with water (2×1.2 L) and brine (2×1.2 L), then dried with anhydrous Na₂SO₄ followed by evaporation under vacuum below 45° C. to afford 705.8 g crude compound of Formula (V-a) (79% purity, 8% 17-β-hydroxy-epimer, 2% 13-epimer).

### Preparations of Aggregates of the Compound of Formula (V-a)

The crude compound of Formula (V-a) was then dissolved in EtOAc (2 L) to form a solution to which were added lithium citrate (241 g, 858.7 mmol) and heptane (450 mL) in sequence to form a mixture. The mixture was stirred at room temperature for about 16 h followed by filtration to afford 434.8 g aggregates of the compound of Formula (V-a) and citrate (99.4% purity, 0.23% 17-β-hydroxy-epimer) and an EtOAc filtrate. The aggregates of the compound of Formula (V-a) and citrate were dissociated/partitioned between EtOAc (1.1 L) and water (1.1 L) and the EtOAc layer was evaporated under vacuum below 45° C. to afford 222.3 g of the compound of Formula (V-a) in a salt free form. The EtOAc filtrate as described herein was further dried under vacuum below 45° C. to form a residue that was then dissolved in EtOAc (650 mL) to form a solution to which was added KOAc (52 g, 530 mmol) and heptane (180 mL) in sequence to form a mixture. The mixture was stirred at room temperature for 16 h followed by filtration to afford 151.4 g aggregates of the compound of Formula (V-a) and acetate (98.5% purity, 0.51% 17-β-hydroxy-epimer, 0.08% 13-epimer).

### Preparation of the Compound of Formula (V-a) in a Salt Free Form

The aggregates of the compound of Formula (V-a) and acetate were dissociated/partitioned between EtOAc (500 mL) and water (500 mL) to afford 91.1 g of the compound of Formula (V-a) in a salt free form. The total yield of the compound of Formula (V-a) in the salt free form is 62% yield by combining the compound of Formula (V-a) prepared from both the citrate and KOAc aggregates.

¹H NMR (400 MHZ, CDCl₃) δ 7.01 (d. J=8.6 Hz, 2H), 6.68 (s, 2H), 4.28 (s, 1H), 4.25 (s, 2H), 4.04-3.80 (m, 4H), 3.69 (dd, J=12.0, 6.7 Hz, 1H), 2.91 (s, 6H), 2.34-1.99 (m, 5H), 1.91-1.79 (m, 6H), 1.66 (dd, J=15.5, 5.4 Hz, 2H), 1.58-1.41 (m, 5H), 1.12 (s, 3H), 0.92 (td, J=13.3, 4.7 Hz, 1H).

### Example 3b: Synthesis of Compound of Formula (V-a), (5R,11R,13R,14S,17R)-11-(4-(dimethylamino)phenyl)-17-(3-hydroxyprop-1-yn-1-yl)-13-methyl-1,2,6,7,8,11,12,13,14,15,16,17-dodecahydrospiro[cyclopenta[a]phenanthrene-3,2′-[1,3]dioxolane]-5,17(4H)-diol

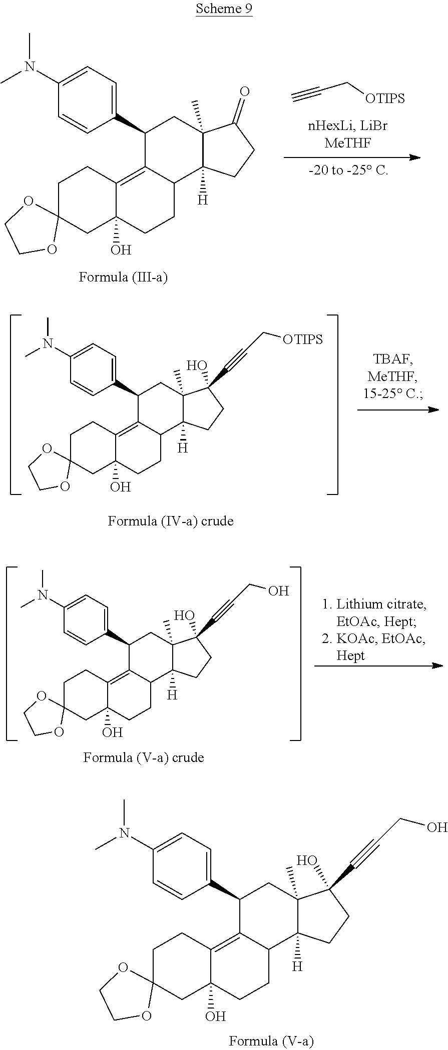

### Preparation of the compound of Formula (V-a), (5R,11R,13R,14S,17R)-11-(4-(dimethylamino)phenyl)-13-methyl-17-(3-((triisopropylsilyl)oxy)prop-1-yn-1-yl)-1,2,6,7,8,11,12,13,14,15,16,17-dodecahydrospiro[cyclopenta[a]phenanthrene-3,2′-[1,3]dioxolane]-5,17(4H)-diol

A solution of O-triisopropylsilyl propargyl alcohol (56.86 g, 0.266 mol), LiBr (23.3 g, 0.269 mol) and MeTHF 1.05 L) was cooled to about −25˜−20° C. To the cooled solution, was dropwise added nHexLi solution (2.2 mol/L in hexane, 120 mL, 0.264 mol) below −10° C. and then stirred for 15˜20 min to form a mixture. To the mixture, was dropwise added a solution of the compound of Formula (III-a) (150 mL MeTHF, 30 g of the compound of Formula (III-a), 66.4 mmol) below −20° C. After addition of the compound of Formula (III-a) solution, the reaction mixture was stirred between −25˜−20° C. for 4.5 h. The reaction mixture was then quenched and washed by ice water (4.5 L). The organic phase was then washed with water (0.3 L) and brine (0.3 L) to afford a solution of compound of Formula (IV-a). Then 85 wt % tetrabutylammonium fluoride (42.03 g, 0.137 mol) was added to the solution of compound of Formula (IV-a) and stirred at 15˜25° C. for 16 h. After complete reaction, the reaction mixture was washed by water (twice, 0.3 L each time) and brine (0.3 L), then was evaporated under vacuum below 45° C. to afford crude the compound of Formula (V-a).

### Preparations of the Compound of Formula (V-a) in a Salt Free Form

The crude compound of Formula (V-a) was then dissolved in solvent mixture of EtOAc (42 mL) and heptane (30 mL) to form a solution to which was added lithium citrate (18.3 g). The mixture was stirred at room temperature for about 16 h followed by filtration and rinse of the cake with a solvent mixture of EtOAc (7.5 mL) and heptane (1.5 mL) to afford aggregates of the compound of Formula (V-a) and citrate (98.0% purity, 1.05% 17-β-hydroxy-epimer) and an EtOAc filtrate. The aggregates of the compound of Formula (V-a) and citrate were dissociated/partitioned between EtOAc (120 mL) and water (120 mL) and the EtOAc layer was evaporated under vacuum below 45° C. to afford 18.26 g of the compound of Formula (V-a) in a salt free form (Purified Crop 1, 60.9% yield based on the compound of Formula IV-a). The EtOAc filtrate as described herein was further dried under vacuum below 45° C. to form a residue that was then dissolved in a solvent mixture of EtOAc (42 mL) and heptane (10 mL) to form a solution to which was added KOAc (3.52 g) in sequence to form a mixture. The mixture was stirred at room temperature for 16 h followed by filtration and rinse of the cake with a solvent mixture of EtOAc (5.1 mL) and heptane (0.9 mL) to afford aggregates of the compound of Formula (V-a) and acetate (Recovered Crop 1). The Recovered Crop 1 was dissociated/partitioned between EtOAc (60 mL) and water (60 mL) and the EtOAc layer was evaporated under vacuum below 45° C. to afford Recovered Crop 2. The Recovered Crop 2 was dissolved in a solvent mixture of EtOAc (42 mL) and heptane (42 mL) to form a solution to which were added lithium citrate (0.33 g) in sequence to form a mixture. The mixture was stirred at room temperature for about 16 h followed by filtration and rinse of the cake with a solvent mixture of EtOAc (5.1 mL) and heptane (0.9 mL) to afford to afford aggregates of the compound of Formula (V-a) and citrate (Recovered Crop 3). The Recovered Crop 3 was dissociated/partitioned between EtOAc (30 mL) and water (30 mL) and the EtOAc layer was evaporated under vacuum below 45° C. to afford 3.09 g of the compound of Formula (V-a) in a salt free form (Purified Crop 2, 10.3% yield based on the compound of Formula IV-a). The total yield of the compound of Formula (V-a) in the salt free form is 71.2% yield based on the compound of Formula IV-a by combining the compound of Formula (V-a) from both Purified Crop 1 and Purified Crop 2.

¹H NMR (400 MHZ, CDCl₃) δ 7.01 (d, J=8.6 Hz, 2H), 6.68 (s, 2H), 4.28 (s, 1H), 4.25 (s, 2H), 4.04-3.80 (m, 4H), 3.69 (dd, J=12.0, 6.7 Hz, 1H), 2.91 (s, 6H), 2.34-1.99 (m, 5H), 1.91-1.79 (m, 6H), 1.66 (dd, J=15.5, 5.4 Hz, 2H), 1.58-1.41 (m, 5H), 1.12 (s, 3H), 0.92 (td, J=13.3, 4.7 Hz, 1H).

### Example 4: Alternative Processes of Preparing Compounds of Formula (V) Via trialkylsilylacetylene Route

### General Procedure D:

Some compounds of Formula (V) can also be prepared from compounds of Formula (III) through the intermediates having the Formula (X) according to the methods outlined in Scheme 9. Alkylation of compounds of Formula (III) with an organometallic reagent of Formula (IX) forms trialkylsilylacetylene intermediates of Formula (IX), which are subject to desilylation to form acetylene intermediates of Formula (XI). The acetylene intermediates of Formula (XI) react with paraformaldehyde in the presence of CuI and at least one base under the suitable conditions as described or provided herein to yield compounds of Formula (V) in crude form. Surprisingly and unexpectedly, the diastereoselectivity for compounds of Formula (V) obtained according to the general process D of Scheme 9 is higher than that obtained from the general process C of Scheme 6. The crude compounds of Formula (V) were purified by forming an aggregate with a suitable salt as described or provided herein and the aggregate was further crystallized in the suitable solvent to produce compounds of Formula (V) in higher purity and diastereoselectivity. The pure compounds of Formula (V) were readily freed from the aggregates by partitioning the aggregates in polar organic solvent and water as described or provided herein. The variables in Scheme 9 are as defined in the embodiments as described herein.

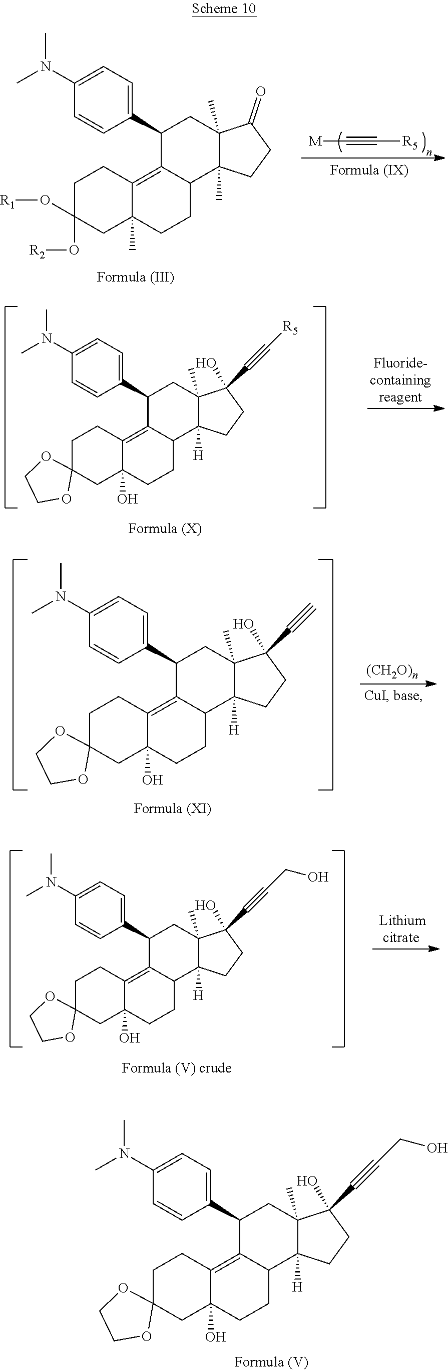

### Example 4a: Alternative Synthesis of the Compound of Formula (V-a), (5R,11R,13R,14S,17R)-11-(4-(dimethylamino)phenyl)-17-(3-hydroxyprop-1-yn-1-yl)-13-methyl-1,2,6,7,8,11,12,13,14,15,16,17-dodecahydrospiro[cyclopenta[a]phenanthrene-3,2′-[1,3]dioxolane]-5,17(4H)-diol

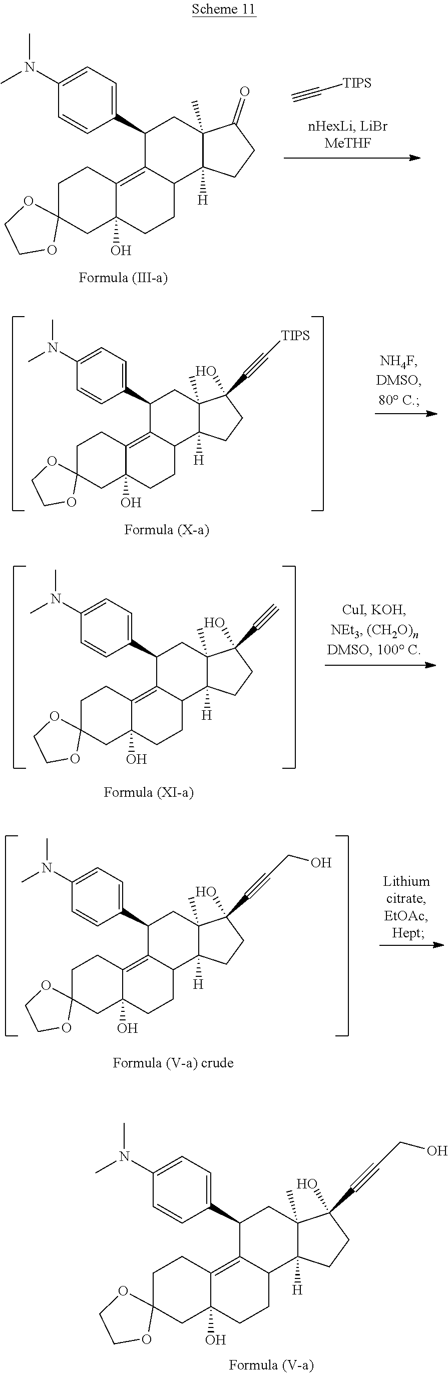

### Preparation of the compound of Formula (X-a), (5R,11R,13R,14S,17R)-11-(4-(dimethylamino)phenyl)-13-methyl-17-((triisopropylsilyl) ethynyl)-1,2,6,7,8,11,12,13,14,15,16,17-dodecahydrospiro[cyclopenta[a]phenanthrene-3,2′-[1,3]dioxolane]-5,17(4H)-diol

A solution of triisopropylsilylacetylene (TIPS-acetylene, 8.48 g, 46.5 mol) and LiBr (4.04 g, 46.5 mol) in MeTHF (50 mL) was cooled to about −70° C. To the cooled solution, was dropwise added a nHexLi solution (2.2 mol/L in hexane, 21.2 mL, 46.5 mmol) over about 1 h to form a mixture. To the resulting mixture, was dropwise added a solution of the compound of Formula (III-a) (20 mL MeTHF, 5 g the compound of Formula (III-a), 11.1 mmol). After the addition of the compound of Formula (III-a) solution, the reaction mixture was warmed to about 35° C. and stirred at that temperature for about 16 h. The reaction mixture was heated to about 50° C., then cooled to room temperature, and stirred at room temperature for about 8 h. HPLC analysis showed that there were 3% the compound of Formula (III-a) remaining, 71% the compound of Formula (IV-a) formed, and 5.3% 17-β-hydroxy-epimer detected with a ratio of the compound of Formula (X-a) to its 17-β-hydroxy-epimer as about 13.4). Surprisingly and unexpectedly, the achieved diastereoselectivity for the compound of Formula (X-a) over its 17-β-hydroxy-epimer herein is much higher than the diastereoselectivity (7:1) for the compound of Formula (IV-A) to its 17-β-hydroxy-epimer obtained according to the general process D of Scheme 7. The reaction mixture was then quenched by ice water (100 mL). The organic phase was washed with water (100 mL) and brine (100 mL) and evaporated under vacuum below 45° C. to give a residue. The residue was purified by silica-gel column chromatography to afford 4.95 g of a mixture of the compound of Formula (X-a) and 17-β-hydroxy-epimer (71% yield). A portion of the mixture was further purified by pre-HPLC for 1H NMR analysis.

1H NMR (400 MHZ, CDCl3) δ 6.95 (d, J=8.7 Hz, 2H), 6.64 (d, J=8.7 Hz, 2H), 4.32 (s, 1H), 4.04-3.82 (m, 4H), 3.69 (dd, J=11.8, 6.4 Hz, 1H), 2.88 (d. J=9.9 Hz, 6H), 2.36-2.08 (m, 4H), 2.02-1.62 (m, 10H), 1.46 (ddt, J=12.2, 7.6, 6.2 Hz, 4H), 1.12 (s, 3H), 1.05-0.98 (m, 21H).

### Preparation of the compound of Formula (XI-a), (5R,11R,13R,14S,17S)-11-(4-(dimethylamino)phenyl)-17-ethynyl-13-methyl-1,2,6,7,8,11,12,13,14,15,16,17-dodecahydrospiro[cyclopenta[a]phenanthrene-3,2′-[1,3]dioxolane]-5,17(4H)-diol with TBAF

TBAF solution (1 mol/L in THF, 1.18 mL, 1.18 mmol) was added dropwise to a solution of the compound of Formula (X-a) and its 17-β-hydroxy-epimer (0.5 g, 0.789 mmol) and DMSO (5 mL) at room temperature and the resulting mixture was stirred for about 16 h. After reaction completion, as indicated by TLC, the reaction mixture was poured into 30 mL ice water and extracted with ethyl acetate (2×30 mL). The organic phases were combined and washed with water (50 mL) and brine (2×50 mL), dried over anhydrous Na₂SO₄, and evaporated under vacuum below 45° C. to give a residue. The residue was purified by silica-gel column chromatography to afford 365 mg of the compound of Formula (XI-a) in 97% yield. A portion of the mixture was further by pre-HPLC for purified 1H NMR analysis.

¹H NMR (400 MHZ, CDCl₃) δ 7.00 (d. J=8.7 Hz, 2H), 6.65 (d, J=8.7 Hz, 2H), 4.30 (s, 1H), 4.02-3.81 (m, 4H), 3.69 (dd, J=11.9, 6.6 Hz, 1H), 2.90 (s, 6H), 2.44 (s, 1H), 2.36-2.10 (m, 4H), 1.95-1.80 (m, 6H), 1.64 (d. J=6.9 Hz, 2H), 1.58-1.39 (m, 4H), 1.13 (s, 3H), 0.95 (td, J=13.4, 4.8 Hz, 1H).

Preparation of the Compound of Formula (XI-a) with NH₄F

NH₄F (58.4 mg, 1.58 mmol) was added to a mixture of the compound of Formula (X-a) and 17-β-hydroxy-epimer (0.5 g, 0.789 mmol) and DMSO (5 mL) at room temperature before being heated to about 80° C. and stirred for about 16 h. After completion of the reaction as monitored by TLC, the reaction mixture was poured into 30 mL ice-water and extracted with ethyl acetate (2×30 mL). The organic phases were combined and washed with water (50 mL) and brine (2×50 mL), dried over anhydrous Na₂SO₄ and evaporated under vacuum below 45° C. to give a residue. The residue was purified by silica column chromatography to afford 358 mg of material as a mixture of the compound of Formula (XI-a) and 17-β-hydroxy-epimer (95% yield).

Preparation of the Compound of Formula (XI-a) with KH₂F

KH₂F (94.8 mg, 1.58 mmol) was added to a mixture of the compound of Formula (X-a) and 17-β-hydroxy-epimer (0.5 g, 0.789 mmol) and DMSO (5 mL) at room temperature before being heated to about 80° C. and stirred for about 16 h. After completion of the reaction as indicated by TLC, the reaction mixture was poured onto 30 mL ice water and extracted with ethyl acetate (2× 30 mL). The organic phases were combined and washed with water (50 mL) and brine (2×50 mL) before being dried with anhydrous Na₂SO₄ and solvent removal under vacuum below 45° C. The residue was purified by silica-gel column chromatography to afford 351 mg of material as a mixture of the compound of Formula (XI-a) and 17-β-hydroxy-epimer (93% yield).

Preparation of the Compound of Formula (XI-a) with KF

KF (91.6 mg, 1.58 mmol) was added to a mixture of the compound of Formula (X-a) and 17-β-hydroxy-epimer (0.5 g, 0.789 mmol) and DMSO (5 mL) at room temperature before being heated to about 80° C. and stirred about for 16 h. After completion of the reaction as indicated by TLC, the reaction mixture was poured into 30 mL ice-water and extracted with ethyl acetate (2×30 mL). The organic phases were combined and washed with water (50 mL) and brine (2×50 mL), dried over anhydrous Na₂SO₄ and evaporated under vacuum below 45° C. to give a residue. The residue was purified by silica column chromatography to afford 340 mg of material as a mixture of the compound of Formula (XI-a) and 17-β-hydroxy-epimer (90% yield).

### Preparation of the Crude Compound of Formula (V-a)

Triethylamine (106 mg, 1.05 mmol) was added to a mixture of the compound of Formula (XI-a) and 17-β-hydroxy-epimer (0.5 g, 1.05 mmol), CuI (20 mg, 0.105 mmol), KOH (118 mg, 2.09 mmol) and paraformaldehyde (314 mg, 10.5 mmol) in DMSO (5 mL) and the resulting mixture was heated to about 100° C. and stirred for about 24 h. After completion of the reaction (the compound of Formula (XI-a)≤3%), the reaction mixture was poured into 10 mL ice water and extracted with ethyl acetate (3× 10 mL). The organic phases were combined and washed with water (10 mL) and brine (10 mL) respectively and evaporated under vacuum below 45° C. to give a residue that was purification by silica column chromatography to afford 490 mg crude the compound of Formula (V-a) (92% yield, the compound of Formula (V-a)/17-β-hydroxy-epimer=15.2).

### Preparations of Aggregates of the Compound of Formula (V-a) and Lithium Citrate

Lithium citrate (300 mg; 1.2 eq) was added to a mixture of the crude compound of Formula (V-a) (500 mg, 74% purity, the compound of Formula (V-a)/17-β-hydroxy-epimer=14.8) and ethyl acetate (2.5 mL), then stirred at about 5 to about 15° C. for about 16 h to form a suspension. The suspension is filtered to give a filter cake. The filter cake was dried under vacuum below 45° C. to afford 425 mg complex of the compound of Formula (V-a) and citrate (60% yield, 95% purity, the compound of Formula (V-a)/17-β-hydroxy-epimer=207).

### Preparation of the Crude Compound of Formula (V-a)

Triethylamine (106 mg, 1.05 mmol) was added to a mixture of the compound of Formula (XI-a) and 17-β-hydroxy-epimer (0.5 g, 1.05 mmol), CuI (20 mg, 0.105 mmol), KOH (118 mg, 2.09 mmol) and paraformaldehyde (314 mg, 10.5 mmol) in DMSO (5 mL) and the resulting mixture was heated to about 100° C. and stirred for about 24 h. After completion of the reaction (the compound of Formula (XI-a)≤3%), the reaction mixture was poured into 10 mL ice water and extracted with ethyl acetate (3×10 mL). The organic phases were combined and washed with water (10 mL) and brine (10 mL) respectively and evaporated under vacuum below 45° C. to give a residue that was purification by silica column chromatography to afford 490 mg crude the compound of Formula (V-a) (92% yield, the compound of Formula (V-a)/17-β-hydroxy-epimer=15.2).

### Preparations of Aggregates of the Compound of Formula (V-a) and Lithium Citrate

Lithium citrate (300 mg; 1.2 eq) was added to a mixture of the crude compound of Formula (V-a) (500 mg, 74% purity, the compound of Formula (V-a)/17-β-hydroxy-epimer=14.8) and ethyl acetate (2.5 mL), then stirred at about 5 to about 15° C. for about 16 h to form a suspension. The suspension is filtered to give a filter cake. The filter cake was dried under vacuum below 45° C. to afford 425 mg complex of the compound of Formula (V-a) and citrate (60% yield, 95% purity, the compound of Formula (V-a)/17-β-hydroxy-epimer=207).

### Example 5: Processes of Preparing Onapristone (Formula (VII)

### General Procedure E:

Onapristone can also be prepared via hydrogenation of compounds of Formula (V) to form intermediates of Formula (VI) followed by hydrolysis of the compounds of Formula (VI) according to the methods outlined in Scheme 12. The variables in Scheme 12 are as defined in the embodiments as described herein.

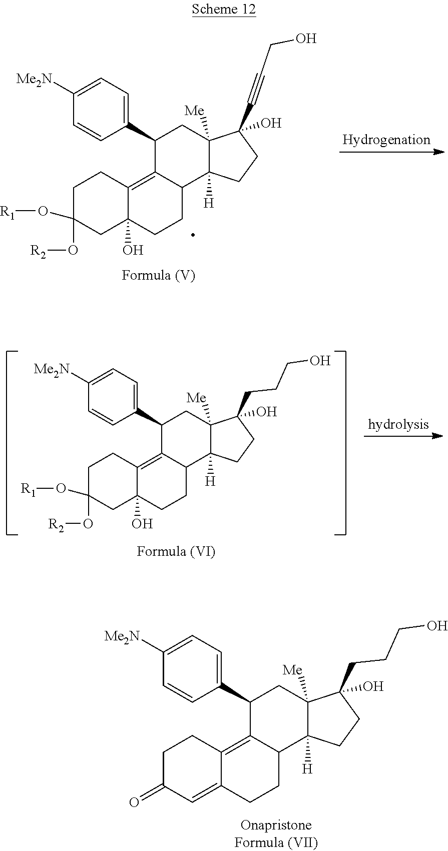

### Example 5a: Synthesis of Onapristone, (8S,11R,13R,14S,17S)-11-[4-(Dimethylamino)phenyl]-17-hydroxy-17-(3-hydroxypropyl)-13-methyl-1,2,6,7,8,11,12,14,15,16-decahydrocyclopenta[a]phenanthren-3-one

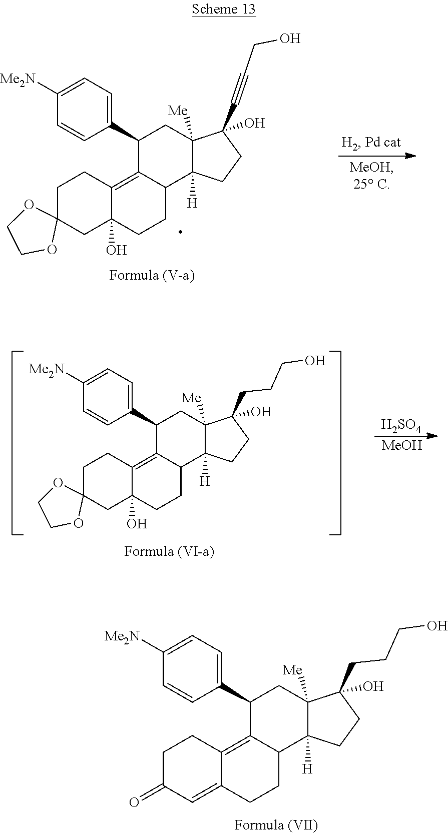

### Preparation of the compound of Formula (VI-a), (5R,11R,13R,14S,17S)-11-(4-(dimethylamino)phenyl)-17-(3-hydroxypropyl)-13-methyl-1,2,6,7,8,11,12,13,14,15,16,17-dodecahydrospiro[cyclopenta[a]phenanthrene-3,2′-[1,3]dioxolane]-5,17(4H)-diol

10% Pd—C(50% wetted, 26 g) was added to a solution of the compound of Formula (V-a) (260 g, 0.51 mol) and pyridine (260 mL) in MeOH (2.6 L) in a reaction vessel and the resulting mixture was then subject to hydrogenation under 1 MPa pressure for about 6.5 h. After completion of the reaction as indicated by HPLC, the mixture was filtered and the filtrate was evaporated under vacuum below 45° C. to give a residue. The residue was dissolved in EtOAc (520 mL) and heptane (780 mL) and the solution was evaporated under vacuum below 45° C. to afford crude the compound of Formula (VI-a). The crude compound of Formula (VI-a) was purified with a silica plug (5× silica, 70V EtOAc as eluent) to afford the compound of Formula (VI-a) (237 g, 90% yield) as white solid.

1H NMR (400 MHZ, CDCl₃) δ 7.00 (d, J=7.7 Hz, 2H), 6.65 (d, J=7.8 Hz, 2H), 5.35 (s, OH), 4.26 (s, 1H), 3.92 (ddd, J=27.3, 14.2, 7.1 Hz, 4H), 3.73-3.55 (m, 1H), 2.90 (s, 6H), 2.20 (ddd, J=50.6, 23.5, 11.9 Hz, 4H), 1.97-1.77 (m, 4H), 1.63 (d, J=8.1 Hz, 3H), 1.56 (d, J=17.1 Hz, 3H), 1.39-1.23 (m, 7H), 1.05 (s, 3H), 0.97-0.80 (m, 4H).

### Preparation of Onapristone

The compound of Formula (VI-a) (235 g, 0.46 mol) was dissolved in MeOH (1.2 L) to form a solution and the solution was cooled to about 0 to about 10° C., then a solution of the diluted sulfuric acid aqueous (70 g 98% concentrated sulfuric acid and 70 mL water, 0.97 mol) was added dropwise to the solution and the temperature of the solution was kept between about 0 to about 5° C. followed by addition of water (235 mL). After the reaction mixture was stirred for about 16 h. to the mixture was slowly added 28% ammonia water (235 mL) and the temperature of the mixture was kept between 0˜ 5° C. Then, the mixture was warmed up to room temperature and water (1.2 L) was added to the mixture and then the mixture was extracted with CH₂Cl₂ (3×1 L). The combined organic layers were washed with water (1.2 L), dried over anhydrous Na₂SO₄ and evaporated under vacuum below 45° C. to afford 212.4 g onapristone, the compound of Formula (VII) (88% yield).

### ¹H NMR (400 MHZ, CDCl₃) δ 7.01 (d. J=7.9 Hz, 2H), 6.67 (d, J=7.7 Hz, 2H), 5.69 (s, 1H), 3.82-3.72 (m, 1H), 3.66 (dd, J=13.0, 6.4 Hz, 2H), 2.92 (s, 6H), 2.62-1.98 (m, 9H), 1.91-1.33 (m, 14H), 1.02 (s, 3H)

### Example 5b: Synthesis of onapristone, (8S,11R,13R,14S,17S)-11-[4-(Dimethylamino)phenyl]-17-hydroxy-17-(3-hydroxypropyl)-13-methyl-1,2,6,7,8,11,12,14,15,16-decahydrocyclopenta[a]phenanthren-3-one

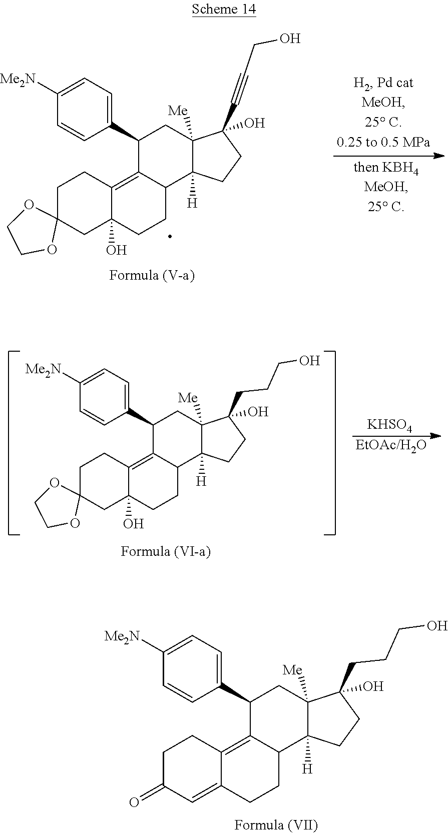

### Preparation of the compound of Formula (VI-a), (5R,11R,13R,14S,17S)-11-(4-(dimethylamino)phenyl)-17-(3-hydroxypropyl)-13-methyl-1,2,6,7,8,11,12,13,14,15,16,17-dodecahydrospiro[cyclopenta[a]phenanthrene-3,2′-[1,3]dioxolane]-5,17(4H)-diol

10% Pd—C(50% wetted, 2 g) was added to a solution of the compound of Formula (V-a) (20 g, 39.4 mol) and triethylamine (20 mL) in MeOH (200 mL) in a reaction vessel and the resulting mixture was then subject to hydrogenation under 0.18 MPa pressure for 18 h. After completion of the reaction as indicated by HPLC, the mixture was filtered and rinsed with methanol (50 mL), and KBH₄ (4 g, 74.8 mmol) was added to form a reaction mixture. Then the reaction mixture was stirred at room temperature for 3 h. After complete reaction indicated by HPLC, water (100 mL) was added and stirred for 30 min. Most of methanol was then evaporated under vacuum to give a residue. EtOAc (200 mL) was added to the residue to extract the product. The EtOAc phase was kept, and the water phase was back extracted with EtOAc twice (60 mL for each). The EtOAc phases were combined and washed with 9.1% wt aqueous NaCl solution (thrice, 100 mL for each), which was further evaporated under vacuum below 45° C. to afford 17.8 g of crude the compound of Formula (VI-a), 88.3% yield. ¹H NMR (400 MHZ, CDCl₃) δ 7.00 (d, J=7.7 Hz, 2H), 6.65 (d, J=7.8 Hz, 2H), 5.35 (s, OH), 4.26 (s, 1H), 3.92 (ddd, J=27.3, 14.2, 7.1 Hz, 4H), 3.73-3.55 (m, 1H), 2.90 (s, 6H), 2.20 (ddd, J=50.6, 23.5, 11.9 Hz, 4H), 1.97-1.77 (m, 4H), 1.63 (d, J=8.1 Hz, 3H), 1.56 (d, J=17.1 Hz, 3H), 1.39-1.23 (m, 7H), 1.05 (s, 3H), 0.97-0.80 (m, 4H).

### Preparation of Onapristone

The crude compound of Formula (VI-a) (2.0 g, 3.91 mmol) was dissolved in EtOAc (20 mL) and a solution of KHSO₄ (1.6 g, 11.7 mmol) in water (8 mL) was added at room temperature. The resulting mixture was stirred at room temperature for 1.5˜2 h, and then added water (10 mL) and 28% ammonia water (2 mL), which was further stirred at room temperature for 0.5˜1 h. After complete conversion indicated by HPLC analysis, the reaction mixture was washed with 9.1% aqueous NaCl solution twice (20 mL for each). Then the EtOAc phase was concentrated under vacuum below 45° C. until no continuous drops could be observed, which give a residue. The residue was added to a mixture solvent of methanol (20 mL), water (4 mL) and 28% ammonia water (1 mL), which was stirred at room temperature for 0.5˜1 h. Then water (20 mL) was added and kept stirring for 10˜20 min, followed by filtration and rinse of cake with water until neutral to afford 1.37 g of the compound of Formula (VII) (Crop 1). The filtrate was extracted with EtOAc twice (20 mL for each), followed by concentration under vacuum to afford 0.38 g of the compound of Formula (VII) (Crop 2). The combine yield of Crop 1 and Crop 2 was 99.4%

### Example 6: Purification of Onapristone by Crystallization from Ethyl Acetate

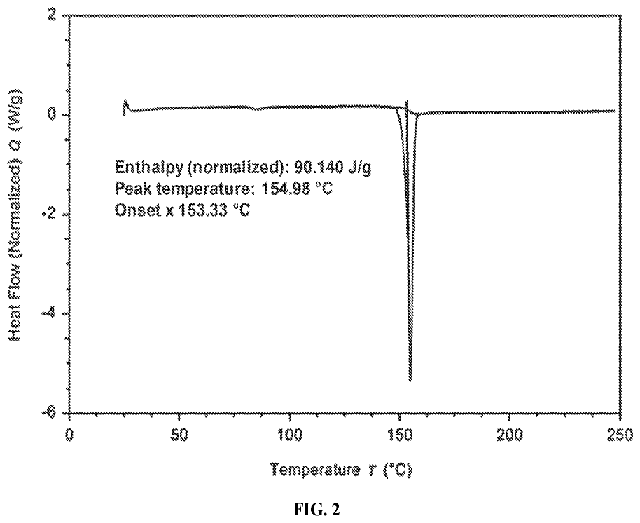

Crude Onapristone, the compound of Formula (VI-a), prepared according to the methods described herein, was further purified by the crystallization process described herein. Crude Onapristone (210 g, 91.7% purity) was recrystallized from ethyl acetate (5 volume of the crude onapristone) (5V) via a standard crystallization process in which the temperature of the saturated solution was varied to produce cake 1 (114 g, 98.2% purity). Cake 1 was recrystallized in ethyl acetate (5 V) with the same process as described herein to produce onapristone in a crystalline form (95 g, 99.1% purity, 54% yield). DSC analysis of this crystalline form shows an exothermic peak at 155° C. as shown in FIG. 2, which is consistent with that of the onapristone crystalline Form A of U.S. Pat. No. 9,193,757, which is incorporated by reference in its entirety. The mother liquids from the two crystallization processes were combined and evaporated to produce the crude onapristone that was dissolved in dichloromethane (5 V) to from a solution. The solution was passed through a 0.1× silica, filtered and rinsed with ethyl acetate (20 V). The liquid was reduced and recrystallized twice from ethyl acetate following the process as described herein to recover onapristone (53 g, 98.6% purity, 31% yield). The combined yield of onapristone was 85%.

### Example 7: Final Crystallization of Onapristone from Ethyl Acetate and n-Heptane

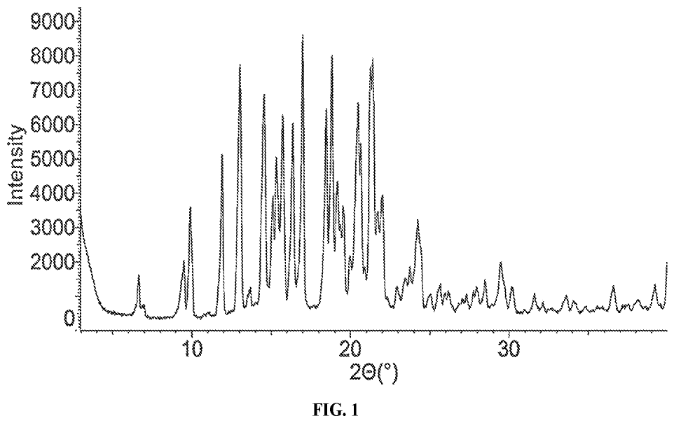

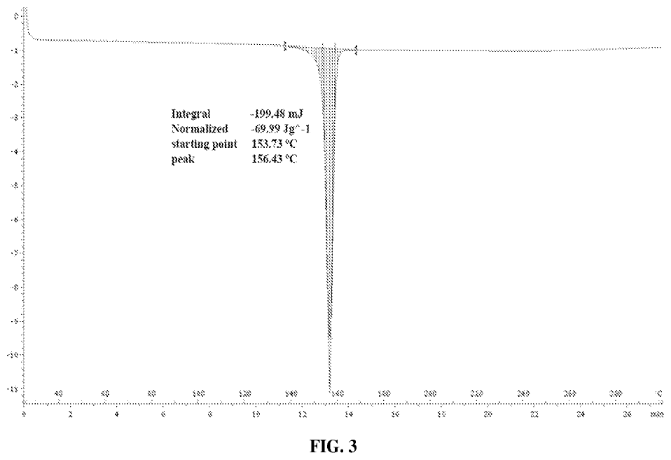

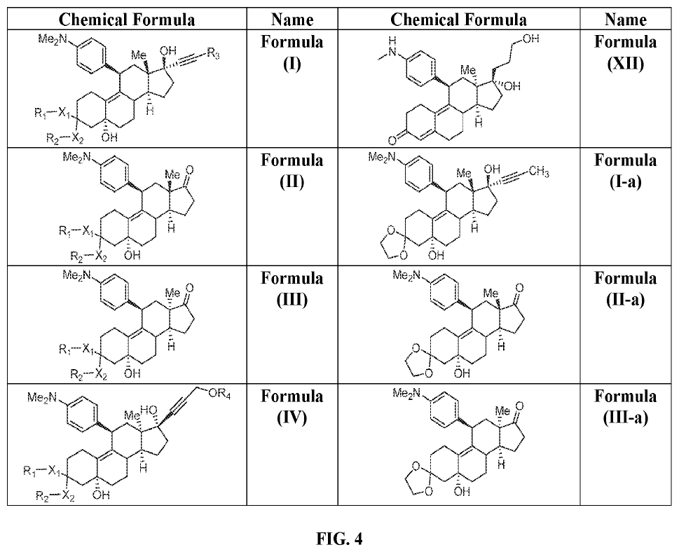

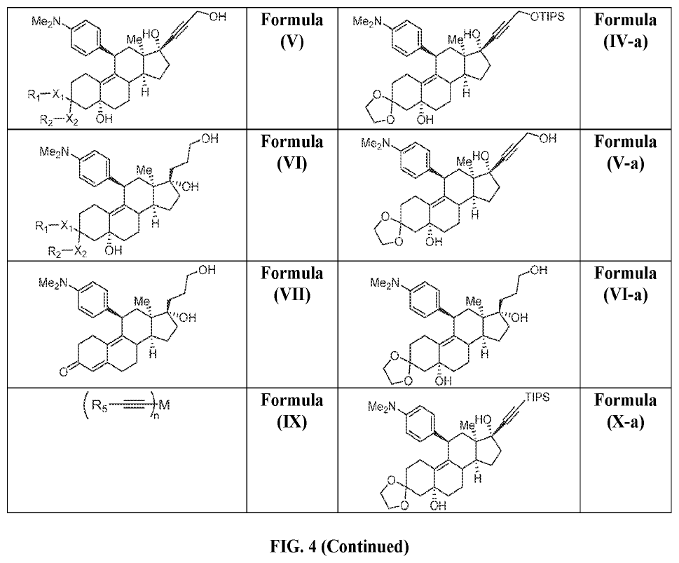

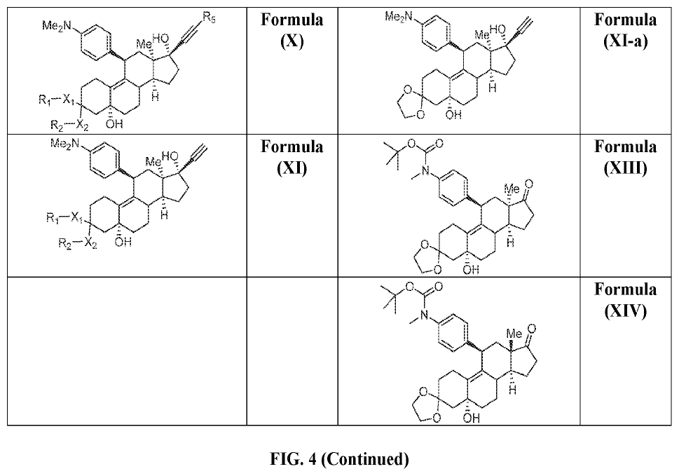

Onapristone (560 g, 98.8% purity) was crystallized from ethyl acetate/n-heptane (1:3 v/v, 8.4 L) following the process as described herein to produce 539 g of onapristone in a crystalline form (99.3% purity, 96% yield). A mixture of the onapristone provided herein and ethyl acetate (5V) was heated to reflux with the inner temperature at about 80° C. to form a saturated solution. To the saturated solution, was added heptane 15 (V) of were added slowly in 5 h while inner temperature was kept between about 75° C. to about 80° C. After completion of the addition of heptane, the mixture was cooled to 25° C. over 8 h and stirred at 25° C. for additional 8 h before filtration to afford onapristone in a crystalline form. FIG. 1 shows the X-ray powder diffraction pattern for the crystalline form crystallized from ethyl acetate/n-heptane and peak positions are provided in Table 1. XRPD analysis showed that the crystalline form of onapristone prepared herein is Form A as described in U.S. Pat. No. 9,193,757, which is incorporated by reference in its entirety, given that the X-ray powder diffraction pattern for the crystalline form prepared herein is consistent with that of the onapristone crystalline Form A of U.S. Pat. No. 9,193,757. DSC analysis of this sample shows an exothermic peak at 155° C. as shown in FIG. 3, which is consistent with that of the onapristone crystalline Form A of U.S. Pat. No. 9,193,757. ¹H NMR (400 MHZ, CDCl₃) δ 7.01 (d. J=7.9 Hz, 2H), 6.67 (d. J=7.7 Hz, 2H), 5.69 (s, 1H), 3.82-3.72 (m, 1H), 3.66 (dd. J=13.0, 6.4 Hz, 2H), 2.92 (s, 6H), 2.62-1.98 (m, 9H), 1.91-1.33 (m, 14H), 1.02 (s, 3H)

TABLE 1

| | | Net | Gross | Rel. |
|---|---|---|---|---|
| Angle | d Value | Intensity | Intensity | Intensity |
| 6.665° | 13.25077 Å | 1173 | 1579 | 14.8% |
| 6.878° | 12.84200 Å | 307 | 701 | 3.9% |
| 9.491° | 9.31129 Å | 1603 | 1983 | 20.2% |
| 9.906° | 8.92224 Å | 3207 | 3586 | 40.5% |
| 11.047° | 8.00285 Å | 66.4 | 483 | 0.8% |
| 11.899° | 7.43150 Å | 4640 | 5111 | 58.6% |
| 13.042° | 6.78269 Å | 7147 | 7696 | 90.2% |
| 13.604° | 6.50394 Å | 590 | 1172 | 7.4% |
| 14.562° | 6.07813 Å | 6229 | 6854 | 78.6% |
| 15.108° | 5.85942 Å | 3254 | 3897 | 41.1% |
| 15.361° | 5.76362 Å | 4093 | 4742 | 51.7% |
| 15.719° | 5.63316 Å | 5587 | 6242 | 70.5% |
| 16.363° | 5.41278 Å | 5339 | 5999 | 67.4% |
| 16.996° | 5.21261 Å | 7923 | 8582 | 100.0% |
| 17.706° | 5.00512 Å | 77.5 | 744 | 1.0% |
| 18.485° | 4.79594 Å | 5715 | 6414 | 72.1% |
| 18.826° | 4.70984 Å | 7252 | 7963 | 91.5% |
| 19.191° | 4.62105 Å | 3613 | 4332 | 45.6% |
| 19.531° | 4.54140 Å | 2884 | 3610 | 36.4% |
| 19.966° | 4.44357 Å | 1378 | 2109 | 17.4% |
| 20.467° | 4.33572 Å | 5872 | 6604 | 74.1% |
| 21.397° | 4.14950 Å | 7165 | 7887 | 90.4% |
| 21.730° | 4.08648 Å | 2753 | 3467 | 34.7% |
| 21.972° | 4.04202 Å | 3153 | 3860 | 39.8% |
| 22.258° | 3.99085 Å | 245 | 943 | 3.1% |
| 22.945° | 3.87285 Å | 605 | 1289 | 7.6% |
| 23.457° | 3.78938 Å | 831 | 1506 | 10.5% |
| 23.743° | 3.74446 Å | 1190 | 1859 | 15.0% |
| 24.231° | 3.67013 Å | 2565 | 3218 | 32.4% |
| 25.018° | 3.55647 Å | 412 | 1030 | 5.2% |
| 25.627° | 3.47335 Å | 670 | 1280 | 8.5% |
| 25.960° | 3.42953 Å | 477 | 1089 | 6.0% |
| 26.188° | 3.40018 Å | 540 | 1153 | 6.8% |
| 26.825° | 3.32083 Å | 171 | 785 | 2.2% |
| 27.076° | 3.29062 Å | 251 | 866 | 3.2% |
| 27.313° | 3.26259 Å | 427 | 1044 | 5.4% |
| 27.817° | 3.20461 Å | 408 | 1023 | 5.1% |
| 27.955° | 3.18916 Å | 646 | 1259 | 8.2% |
| 28.478° | 3.13171 Å | 851 | 1457 | 10.7% |
| 29.470° | 3.02852 Å | 1426 | 2003 | 18.0% |
| 30.194° | 2.95754 Å | 718 | 1261 | 9.1% |
| 30.991° | 2.88331 Å | 95.4 | 618 | 1.2% |
| 31.603° | 2.82885 Å | 526 | 1059 | 6.6% |
| 32.121° | 2.78437 Å | 299 | 838 | 3.8% |
| 32.638° | 2.74143 Å | 105 | 644 | 1.3% |
| 33.602° | 2.66497 Å | 475 | 1018 | 6.0% |
| 34.104° | 2.62684 Å | 275 | 820 | 3.5% |
| 34.814° | 2.57490 Å | 137 | 685 | 1.7% |
| 35.584° | 2.52093 Å | 152 | 709 | 1.9% |
| 36.578° | 2.45469 Å | 751 | 1305 | 9.5% |
| 37.245° | 2.41222 Å | 169 | 735 | 2.1% |
| 37.451° | 2.39945 Å | 150 | 726 | 1.9% |
| 38.124° | 2.35861 Å | 281 | 885 | 3.5% |
| 39.190° | 2.29688 Å | 688 | 1333 | 8.7% |

### Example 8: Synthesis of the Compound of Formula (XII) Via N-Demethylation of Onapristone

The compound of Formula (XII) or a pharmaceutically acceptable salt thereof is expected to be prepared a) by subjecting the compound of Formula (VII) or a pharmaceutically acceptable salt thereof, as described or provided herein, under a cytochrome P450 3A-mediated N-demethylation, b) by selective mono demethylation of Formula (VII), as described or provided herein, or c) from an N-methyl carbamate derivative of Formula (III), which is expected to be prepared from the N-tert-butyl carbamate N-methyl derivative of Formula (II) according to the photochemistry process as described in Example 2. For example, the compound of N-methyl carbamate derivative of Formula (III) having a formula of

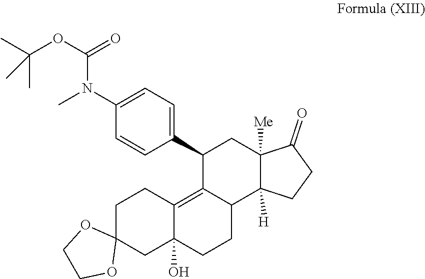 [SMILES: CN(C(=O)OC(C)(C)C)c1ccc([C@H]2C[C@@]3(C)C(=O)CC[C@H]3C3CC[C@@]4(O)CC5(CCC4=C32)OCCO5)cc1]

is expected to be prepared from the N-tert-butyl carbamate N-methyl derivative of Formula (II-a) having a formula of

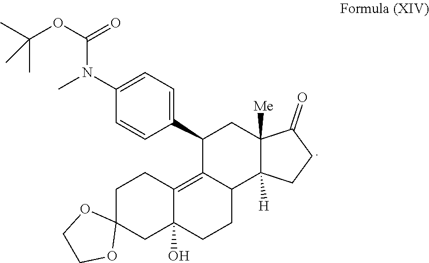 [SMILES: CN(C(=O)OC(C)(C)C)c1ccc([C@H]2C[C@]3(C)C(=O)CC[C@H]3C3CC[C@@]4(O)CC5(CCC4=C32)OCCO5)cc1]

The present embodiments and examples provided herein demonstrate the surprising and unexpected ability to synthesize the compounds provided herein with a better yield, purity, and/or simplified process that can be more cost efficient or other wise saves times or has other benefits that previous synthetic methods did not have.

This specification contains numerous citations to patents, patent applications, and publications, each of which is hereby incorporated by reference for all purposes.

1. A process of preparing the compound of Formula (II), or a pharmaceutically acceptable salt thereof, the process comprising:
contacting the compound of 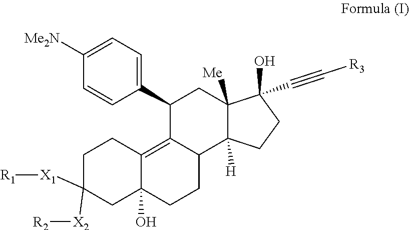
with a base in a first organic solvent under suitable conditions to produce the compound of 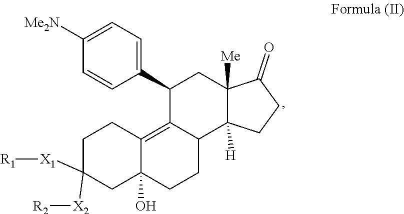
wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₃ is H, optionally substituted C₁-C₆ alkyl, optionally substituted C₁-C₆ hydroxyalkyl, optionally substituted C₁-C₆ alkoxy, optionally substituted cycloalkyl.

2. The process of claim 1, wherein the base is an alkyl metal oxide,
optionally the suitable condition comprises heating the mixture of the compound of Formula (I) and the base in the organic solvent to a temperature;
optionally the temperature is at least about 30° C.;
optionally the first organic solvent is a non-polar organic solvent.

3-4. (canceled)

5. The process of claim 1, further comprising at least one of the following steps:
(1) adding aqueous NaCl solution to the reaction mixture;
(2) cooling the resulting mixture to a temperature between about 0° C. and about 40° C.;
(3) separating the resulting organic phase and washing with aqueous NaCl solution;
(4) evaporating the organic phase to form a residue, optionally the residue is purified by precipitation, optionally the precipitation comprises:
dissolving the residue in a first solvent to form a mixture; optionally the first solvent is an alcohol,
heating the mixture to a temperature; optionally the mixture is heated to a temperature of about 55° C.,
adding a second solvent to the heated mixture to form a precipitate, optionally the second solvent is water, an ether, an alkane, an ester, a ketone, or a combination thereof; and
collecting the precipitate by filtration to produce the compound of Formula (II);
optionally the volume ratio of the second solvent to the first solvent is about 3 to 1.

6-15. (canceled)

16. A process of preparing a compound of Formula (III), or a pharmaceutically acceptable salt thereof, the process comprising:
irradiating a solution of the compound of 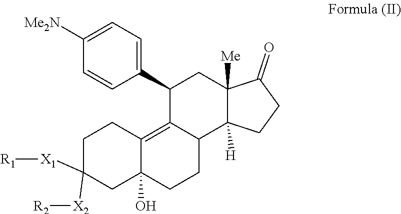
in an organic solvent with a UV light in a flow reactor to produce the compound of 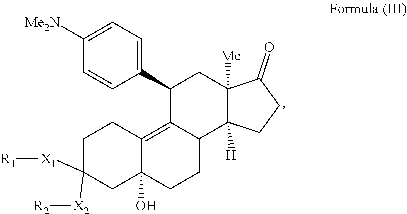
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal.

17. The process of claim 16, wherein
the UV light is a narrow band frequency light at a wavelength from about 300 nm to about 320 nm;
the UV light is from a low-pressure mercury lamp;
the flow reactor is fabricated from a long fluorinated ethylene propylene (FEP) tubing with an inner diameter between 1 to 20 millimeters; and/or
the organic solvent is ethyl acetate, toluene, methyltetrahydrofuran, tetrahydrofuran, isopropyl ether, methanol, water, dichloromethane, isopropylether, or a combination thereof.

18-20. (canceled)

21. The process of 16, further comprising washing the solution with aqueous NaHSO₃ solution to remove the aldehyde impurities from the UV irradiation;
purifying the crude compound of Formula (III) with solid absorbents such as silica gel, magnesium silicate, alumina, polymers, clays, or other porous or high surface area solids; and/or
recrystallizing the compound of Formula (III).

22-23. (canceled)

24. A process of preparing a compound of Formula (X), or a salt thereof, the process comprising:
contacting the compound of 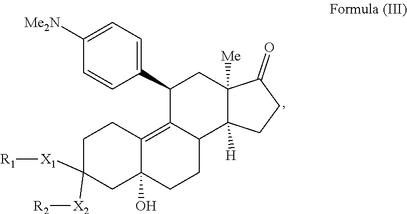
with an organometallic reagent having a formula of 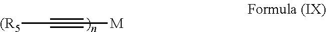
to produce the compound of 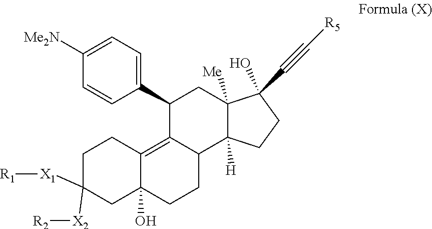
or a salt form thereof; and
wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₅ is trialkylsilyl or triakylsilyloxy, optionally R₅ is trialkylsilyl.

25. The process of claim 24, wherein the organometallic reagent is an organolithium reagent, the organolithium reagent is 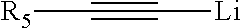
optionally the organolithium reagent is prepared by contacting 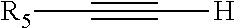
with an alkyllithium reagent in a first organic solvent at a low temperature;
optionally the compound of Formula (III) or a salt form thereof is separately dissolved in a second organic solvent before contacting the organolithium reagent at the low temperature;
optionally both the first and second organic solvents are polar non-protic organic solvents;
optionally the molar ratio of the organolithium reagent to the compound of Formula (III) is between about 1:1 to about 10:1;
optionally a diastereofacial selectivity for the compound of Formula (X) to the 17-β-hydroxy-epimer thereof is achieved.

26-31. (canceled)

32. The process of claim 24, the process further comprising:
contacting the solution of the compound of Formula (X) with a fluoride-containing reagent to produce the compound having a formula of 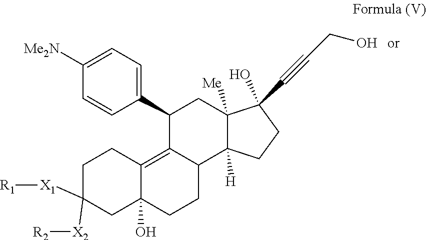 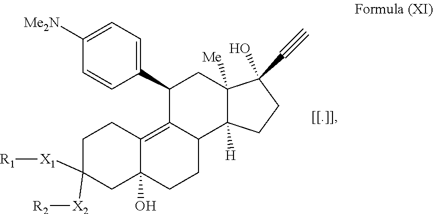
optionally the fluoride-containing reagent is added to the solution of the compound of Formula (X) to form a mixture,
the mixture is optionally washed with water and brine, optionally dried over a drying reagent, and evaporated to form the compound having a formula of Formula (V) or Formula (XI) with the 17-β-hydroxy-epimer thereof as an impurity,
optionally the process further comprises:
contacting the compound of 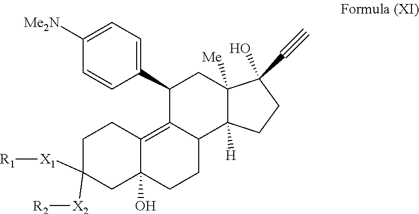
with paraformaldehyde,
at least one bases, and CuI in a fourth organic solvent under suitable conditions to produce the compound of 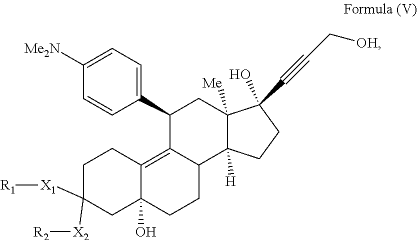
optionally the process further comprises two bases,
optionally the process further comprises purifying the compound of Formula (V) from the 17-β-hydroxy-epimer thereof comprising forming an aggregate of the compound of Formula (V) with a salt,
optionally the process further comprises dissolving the aggregate in the polar non-protic organic solvent to form an organic phase, washing the organic phase with water, and concentrating the organic phase to form the compound of Formula (V) in high purity.

33-37. (canceled)

38. The process of claim 24, wherein the compound of Formula (X) or a salt form thereof, has a formula of 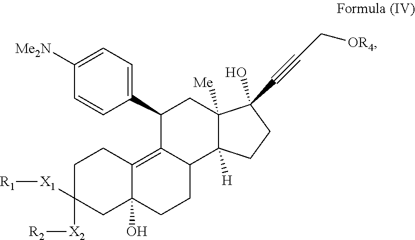
wherein:
X₁ and X₂ are each independently O or S;
M is Li, Na, K, MgBr, CuBr, CuLi, Mg, Cu, or Al;
n is 1-3;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₄ is trialkylsilyl.

39-40. (canceled)

41. A process of preparing a compound of Formula (VII), or a pharmaceutically acceptable salt thereof, the process comprising:
a) subjecting the compound of 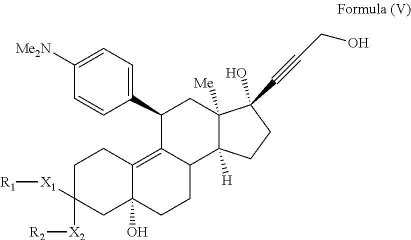
or a salt form thereof, prepared according to the process of claim 24 under a hydrogenation condition to produce the compound of 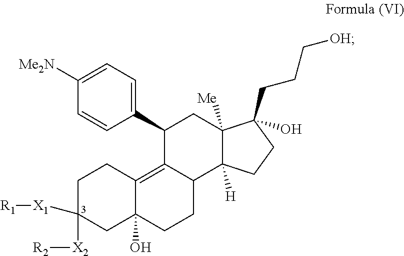
and
e) hydrolyzing the compound of Formula (VI) under a hydrolysis condition to produce the compound of 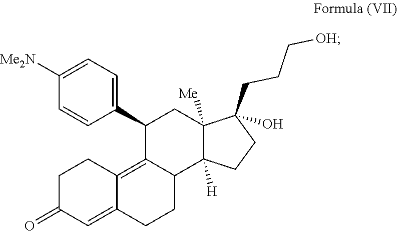 [SMILES: CN(C)c1ccc([C@H]2C[C@]3(C)[C@@H](CC[C@]3(O)CCCO)C3CCC4=CC(=O)CCC4=C32)cc1]
or a pharmaceutically acceptable salt;
optionally the process further comprises: recrystallizing the compound of Formula (VII).

42. The process of claim 41, wherein the hydrogenation condition is suitable for converting an unsaturated carbon-carbon triple bond to a saturated carbon-carbon bond, and/or
the hydrolysis condition is a suitable condition for removing the protection for the 3-ketone of Formula (VI).

43-44. (canceled)

45. A process of preparing a compound of Formula (XII), or a pharmaceutically acceptable salt thereof, the process comprising:
subjecting the compound of 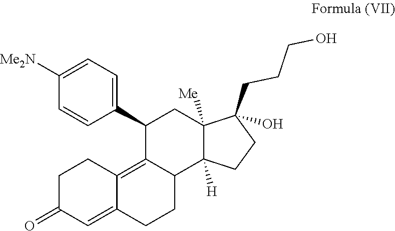 [SMILES: CN(C)c1ccc([C@H]2C[C@]3(C)[C@@H](CC[C@]3(O)CCCO)C3CCC4=CC(=O)CCC4=C32)cc1]
or pharmaceutically acceptable salt thereof, prepared according to the process of claim 41 under a suitable N-demethylation condition to produce the compound of 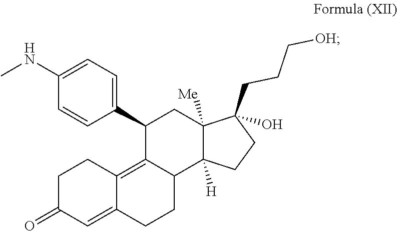 [SMILES: CNc1ccc([C@H]2C[C@]3(C)[C@@H](CC[C@]3(O)CCCO)C3CCC4=CC(=O)CCC4=C32)cc1]
or a pharmaceutically acceptable salt;
optionally the suitable N-demethylation condition is suitable for removing a methyl group from the dimethylamino phenyl group on the compound of Formula (VII).

46. (canceled)

47. A compound having a formula of 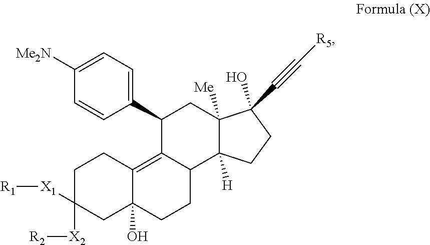
or a pharmaceutically acceptable salt thereof,
wherein:
X₁ and X₂ are each independently O or S;
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal or cyclic thioketal; and
R₅ is trialkylsilyl or triakylsilyloxy.

48. The compound of claim 47, or a pharmaceutically acceptable salt thereof, wherein
(1) both X₁ and X₂ are O;
(2) R₁ and R₂ are each independently optionally substituted C₁-C₆ alkyl, optionally R₁ and R₂ are each independently Me, Et, Pr, or Bu;
(3) R₁, R₂, X₁, and X₂ are together with the carbon atom connected X₁ and X₂ to form optionally substituted 4-, 5-, 6-, 7-, or 8-membered cyclic ketal, optionally R₁, R₂, X₁, and X₂ are together with the carbon atom connected X₁ and X₂ to form optionally substituted 5- or 6-membered cyclic ketal, optionally R₁, R₂, X₁, and X₂ are together with the carbon atom connected X₁ and X₂ to form optionally substituted 5-membered cyclic ketal;
(4) R₅ is trialkylsilyl, optionally R₅ is trimethylsilyl (TMS), triethylsilyl (TES), tert-butyldimethylsilyl (TBS), tert-butyldiphenylsilyl (TBDPS), or triisopropylsilyl (TIPS), optionally R₅ is triisopropylsilyl (TIPS); or
(5) R₅ is triakylsilyloxy, optionally R₅ is trimethylsilyloxy (OTMS), triethylsilyloxy (OTES), tert-butyldimethylsilyloxy (OTBS), tert-butyldiphenylsilyloxy (OTBDPS), or triisopropylsilyloxy (OTIPS), optionally R₅ is triisopropylsilyloxy (OTIPS).

49-56. (canceled)

57. The compound of claim 47, or a pharmaceutically acceptable salt thereof, wherein the compound of Formula (X) has a formula of 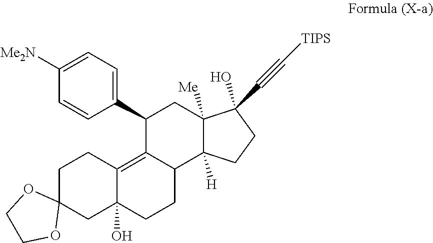
or the compound of Formula (X) has a formula of

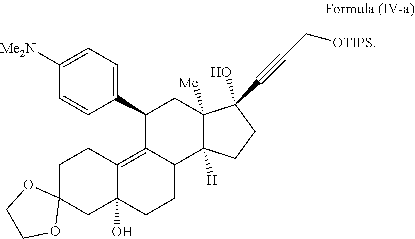

58-61. (canceled)

62. A compound having a formula of 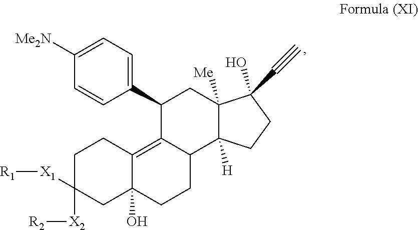
or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.

63. The compound of claim 62, or a pharmaceutically acceptable salt thereof, wherein
(1) both X₁ and X₂ are O;
(2) R₁ and R₂ are each independently optionally substituted C₁-C₆ alkyl; optionally R₁ and R₂ are each independently Me, Et, Pr, or Bu;
(3) R₁, R₂, X₁, and X₂ are together with the carbon atom connected X₁ and X₂ to form optionally substituted 4-, 5-, 6-, 7-, or 8-membered cyclic ketal, optionally R₁, R₂, X₁, and X₂ are together with the carbon atom connected X₁ and X₂ to form optionally substituted 5-membered cyclic ketal; and/or
(4) the compound of Formula (XI) has a formula of

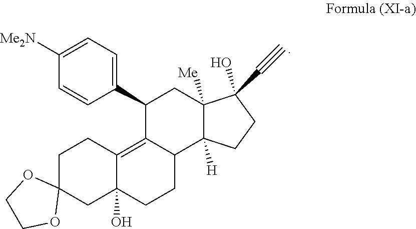 [SMILES: C#C[C@@]1(O)CC[C@H]2C3CC[C@@]4(O)CC5(CCC4=C3[C@@H](c3ccc(N(C)C)cc3)C[C@]21C)OCCO5]

64-68. (canceled)

69. A compound having a formula of 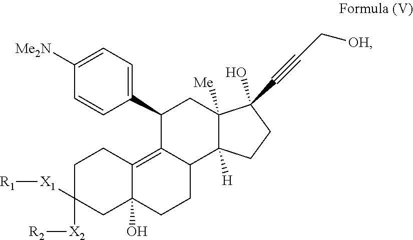
or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.

70. The compound of claim 69, or a pharmaceutically acceptable salt thereof, wherein
(1) both X₁ and X₂ are O;
(2) R₁ and R₂ are each independently optionally substituted C₁-C₆ alkyl, optionally R₁ and R₂ are each independently Me, Et, Pr, or Bu;
(3) R₁, R₂, X₁, and X₂ are together with the carbon atom connected X₁ and X₂ to form optionally substituted 4-, 5-, 6-, 7-, or 8-membered cyclic ketal, optionally R₁, R₂, X₁, and X₂ are together with the carbon atom connected X₁ and X₂ to form optionally substituted 5-membered cyclic ketal;
(4) the compound of Formula (V) has a formula of

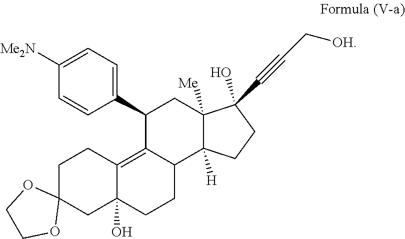 [SMILES: CN(C)c1ccc([C@H]2C[C@]3(C)[C@@H](CC[C@]3(O)C#CCO)C3CC[C@@]4(O)CC5(CCC4=C32)OCCO5)cc1]

71-75. (canceled)

76. A compound having a formula of 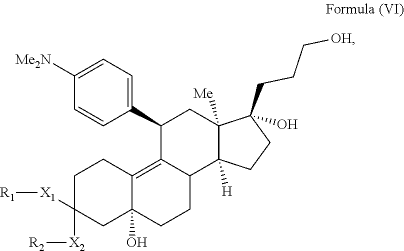
or a pharmaceutically acceptable salt thereof,
wherein:
X₁ and X₂ are each independently O or S; and
R₁ and R₂ are each independently a bond, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₁, R₂, X₁, and X₂ are together with the carbon atom connected X₁ and X₂ to form optionally substituted 4-, 5-, 6-, 7-, or 8-membered cyclic ketal.

77. The compound of claim 76, or a pharmaceutically acceptable salt thereof, wherein
(1) both X₁ and X₂ are O;
(2) R₁ and R₂ are each independently optionally substituted C₁-C₆ alkyl, optionally R₁ and R₂ are each independently Me, Et, Pr, or Bu;
(3) R₁, R₂, X₁, and X₂ are together with the carbon atom connected X₁ and X₂ to form optionally substituted 4-, 5-, 6-, 7-, or 8-membered cyclic ketal, optionally R₁, R₂, X₁, and X₂ are together with the carbon atom connected X₁ and X₂ to form optionally substituted 5-membered cyclic ketal; or
(4) the compound of Formula (VI) has a formula of

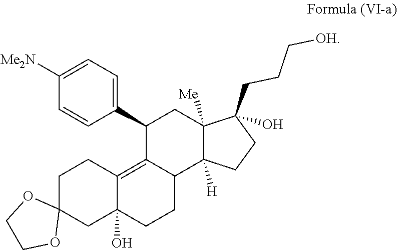 [SMILES: CN(C)c1ccc([C@H]2C[C@]3(C)[C@@H](CC[C@]3(O)CCCO)C3CC[C@@]4(O)CC5(CCC4=C32)OCCO5)cc1]

78-82. (canceled)
